(19) United States
(12) Patent Application Publication
(10) Pub. No.: US 2016/0067272 A1
(43) Pub. Date: Mar. 10, 2016

(54) METHODS FOR TREATING IMMUNE DISEASES

(71) Applicant: THE BRIGHAM AND WOMEN'S HOSPITAL, INC., Boston, MA (US)

(72) Inventor: Abdala EL KHAL, Waltham, MA (US)

(73) Assignee: THE BRIGHAM AND WOMEN'S HOSPITAL, INC., Boston, MA (US)

(21) Appl. No.: 14/783,193

(22) PCT Filed: Apr. 9, 2014
(86) PCT No.: PCT/US2014/033441

§ 371 Date: Oct. 8, 2015

Related U.S. Application Data
(60) Provisional application No. 61/847,290, filed on Jul. 17, 2013.
(60) Provisional application No. 61/809,972, filed on Apr. 9, 2013.

(51) Int. Cl. A61K 31/7084; G01N 33/564

(52) U.S. Cl. CPC A61K 31/7084; G01N 33/564; G01N 2400/00

(57) ABSTRACT

Provided herein are methods for treating or preventing an immune disease in a subject by administering a composition comprising a therapeutically effective amount of NAD+. Also provided herein are methods and assays for diagnosing an immune disease in a subject by measuring the level of NAD+ in a biological sample obtained from the subject.

### FIELD

The invention relates to methods of treating and diagnosing immune diseases with nicotinamide adenine dinucleotide (NAD+).

### BACKGROUND

CD4⁺ helper T (Th) cells play a central role in regulating the adaptive immune response associated with pathogen invasion and numerous diseases including autoimmunity, allergic responses, transplantation as well as tumor immunity¹⁻². TCR activation and stimulation in the periphery in a specific cytokine environment can result in the differentiation of naïve CD4^{|} T cells into distinct lineages of Th cells such as Th1, Th2, Th17 and induced regulatory T cells (iTregs) with distinct functions and non-stochastic cytokine production³. CD4⁺ T cell differentiation was first described in 1986 by Mossman & Coffman showing that CD4⁺ T cells could be divided in two major groups Th1 and Th2⁴⁻⁷. Th1 and Th2 T cell subsets can mainly be distinguished by their cytokine profile, the expression pattern of cell surface molecules and the activation of specific transcription factors. The two other major CD4⁺ T cell subsets, Th17 and iTregs, were characterized recently and were described as distinct lineages from Th1 and Th2⁸⁻¹⁰.

Naïve CD4⁺ T cell differentiation into Th1 cells requires the cytokine IL-12 and is orchestrated through the transcription factors STAT1, STAT4, and Tbx21^{3,11}. Th1 cells have the propensity to produce IFN-γ, IL-2, TNF-α and are known to enhance clearance of intracellular pathogens^{3,11-12}. Under particular conditions, such as chronic Th1 stimulation or high dose antigenic stimulation, Th1 cells can also produce IL-13 and IL-10, two cytokines that have been originally described as Th2 cytokines¹³. It was also shown that co-stimulation of human lymphocytes with CD46, a complement receptor, enhances IL-10 production by IFN-γ-producing Th1 effector cells. These cells were termed regulatory type 1 (Tr1) cells because of their immunosuppressive properties¹⁴⁻¹⁶. Th2 cells which play a cardinal feature in parasitic infections produce IL-4, IL-5, IL-6, IL-10 and IL-13 and require the activation of STAT6, and the transcription factor GATA3³. Th17 cells secrete IL-17A, IL-17F and IL-22 and require the transcription factor RORγt^{2,17}. Th17 cells are involved in host defense against bacteria and fungi². Furthermore, CD4⁻ T cells can be induced in the periphery into CD4⁺ CD25⁺ Foxp3⁺ regulatory T cells and can produce TGF-β, IL-10 and IL-35. These iTregs play a major role in the maintenance of self-tolerance and the prevention of autoimmunity and iTreg induction requires TGF-b, STAT5 and the transcription factor Foxp3^{8,18}.

To differentiate into Th1, Th2, Th17 or iTregs, naïve CD4⁺ T cells require a specific cytokine milieu³. Th1 induction requires the presence of IL-12 while IL-4 induces Th2 differentiation¹². It is well established that IFNγ produced by Th1 cells inhibits Th2 development while IL-4 produced by Th2 cells inhibits Th1 differentiation¹⁹. In addition to TGF-β, which is required for iTreg induction⁸, Th17 cells require IL-6, IL-21 and IL-23 for their differentiation and proliferation, respectively²⁰⁻²⁴. In addition to IFNγ and IL-4, which have been shown to inhibit Th17 differentiation, IL-2 has been shown to block Th17 cell development as well^{2,25-26}. Thus, for the past quarter century it has been considered that cytokine milieu is critical and indispensable for naïve CD4⁺ T cell differentiation^{3,19,27}.

### SUMMARY

The compositions and methods described herein are based, in part, on the novel discovery that nicotinamide adenine dinucleotide (NAD⁺), a cofactor naturally found in the body and secreted during inflammation or under physiological conditions by different cell types such as epithelial cells, fibroblasts and neurons²⁸⁻³⁵, is able to regulate CD4⁺ T cell differentiation. As shown herein, NAD⁻ was able to regulate naïve CD4⁻ T cell differentiation in the absence of exogenous cytokines and more importantly had the capacity to override the effects of Th1, Th2 and iTreg polarizing conditions on T cell differentiation and cytokine production. Although mice treated with NAD+ showed reduced Treg frequency and increased Th17 response, animals were still protected against experimental autoimmune encephalomyelitis (EAE) via a significant rise in Tr1 cells. Surprisingly, NAD+ was identified as a robust therapy since when it was administered after disease onset it had the capacity not only to block but also to reverse EAE progression by promoting myelin and axonal regeneration. Further, the study described herein in the Examples section also demonstrated that NAD+ enhanced tryptophan hydroxylase-1 (Tph-1) expression by CD4+ T cells under Th0, Th1, Th2, and iTreg polarizing conditions. In vivo blockade of Tph-1 altered CD4+ T cell differentiation observed after NAD+ administration and abolished the protective properties of NAD+ against EAE.

As also demonstrated herein in the Examples section, NAD⁺ promotes in vitro, the conversion of human and mice nTregs, a T cell subset that controls autoimmunity and tissue homeostasis, into Th17 cells and their proliferation without addition of exogenous cytokines and in presence of IL-2. In vivo, NAD⁺ enhanced Th17 development and promoted allograft survival independently from nTregs through a robust systemic production of the immunosuppressive IL-10 cytokine. Accordingly, as demonstrated herein, a novel mechanism of nTreg conversion was discovered that is distinct from the “classical cytokine pathway” and provides a rationale for the therapeutic potential of NAD⁺ in transplantation. The working examples also indicate that NAD+ can be used as an effective treatment for allergy and Type 1 diabetes. It is also contemplated herein that analogs of NAD+ can be used with the methods described herein.

Accordingly, provided herein, in some aspects are methods for treating or preventing an immune disease, the method comprising administering a composition comprising a therapeutically effective amount of NAD+ or an analog thereof to a subject in need thereof, thereby treating the immune disease (e.g., transplant rejection, type 1 diabetes, asthma, allergy etc).

In one embodiment of this aspect and all other aspects described herein, wherein the composition further comprises a pharmaceutically acceptable carrier.

In another embodiment of this aspect and all other aspects described herein, the immune disease is selected from the group consisting of Type 1 diabetes, allergy, asthma, eczema, allergy, food allergy, systemic lupus erythematosus, rheumatoid arthritis, transplantation, inflammatory bowel disease, cancer, multiple sclerosis and sepsis. In one embodiment of this aspect and all other aspects described herein, the immune disease is an atopic disorder (e.g., allergy, food allergy, eczema). In another embodiment of this aspect and all other aspects described herein, the immune disease is chronic inflammation, such as chronic obstructive pulmonary disease (COPD). In one embodiment, the immune disease is graft-versus-host disease.

In another embodiment of this aspect and all other aspects described herein, the composition is administered by a route selected from the group consisting of: intravenous, intramuscular, subcutaneous, intradermal, topical, intraperitoneal, intrathecal, intrapleural, intrauterine, rectal, vaginal, intrasynovial, intraorgan, intraocular/periocular, intratumor, and parenteral administration.

In another embodiment of this aspect and all other aspects described herein, the method further comprises a step of diagnosing the subject with an immune disease prior to treatment.

In another embodiment of this aspect and all other aspects described herein, the method further comprises a step of measuring NAD+ in the subject prior to treatment.

In another embodiment of this aspect and all other aspects described herein, the amount of NAD+ is compared to a reference value.

In another embodiment of this aspect and all other aspects described herein, the reference value is obtained from a plurality of subjects in which an immune disease cannot be detected using standard methods.

In another embodiment of this aspect and all other aspects described herein, the reference value is obtained from the subject at an earlier time point.

In another embodiment of this aspect and all other aspects described herein, the earlier time point is prior to the onset of symptoms associated with the immune disease.

In another embodiment of this aspect and all other aspects described herein, treatment with NAD+ or an analog thereof increases systemic IL-10 cytokine production by at least 10% (e.g., at least 20%, at least 30%, at least 40%, at least 50%, at least 60%, at least 70%, at least 80%, at least 90%, at least 1-fold, at least 2-fold, at least 5-fold, at least 10-fold, at least 100-fold or more than the systemic IL-10 cytokine production in the absence of NAD+ or an analog thereof.

Another aspect described herein relates to a method for activating CD4+ helper T cells, the method comprising contacting a CD4+ helper T cell with a composition comprising an effective amount of NAD+ or an analog thereof to a subject in need thereof, thereby activating the CD4+ helper T cell.

Also provided herein, in another aspect, is an assay comprising: a. measuring or quantifying the amount of NAD+ in a biological sample obtained from a subject having or suspected of having an immune disease; and b. comparing the measured or quantified amount of NAD+ with a reference value, and if the amount of NAD+ is decreased relative to the reference value, identifying the subject as having an increased probability of having an immune disease.

In one embodiment of this aspect and all other aspects described herein, the immune disease is selected from the group consisting of Type 1 diabetes, allergy, asthma, eczema, systemic lupus erythematosus, rheumatoid arthritis, transplantation, inflammatory bowel disease, cancer, multiple sclerosis and sepsis. In one embodiment of this aspect and all other aspects described herein, the immune disease is an atopic disorder (e.g., allergy, food allergy, eczema). In another embodiment of this aspect and all other aspects described herein, the immune disease is chronic inflammation (e.g., chronic obstructive pulmonary disease). In one embodiment, the immune disease is graft-versus-host disease.

In another embodiment of this aspect and all other aspects described herein, the assay further comprises a step of treating the subject.

In another embodiment of this aspect and all other aspects described herein, the subject is treated with a composition comprising a therapeutically effective amount of NAD+ or an analog thereof.

In another embodiment of this aspect and all other aspects described herein, treatment with NAD+ or an analog thereof increases systemic IL-10 cytokine production by at least 10% (e.g., at least 20%, at least 30%, at least 40%, at least 50%, at least 60%, at least 70%, at least 80%, at least 90%, at least 1-fold, at least 2-fold, at least 5-fold, at least 10-fold, at least 100-fold or more than the systemic IL-10 cytokine production in the absence of NAD+ or an analog thereof.

In another embodiment of this aspect and all other aspects described herein, the amount of NAD+ is compared to a reference value.

In another embodiment of this aspect and all other aspects described herein, the reference value is obtained from a plurality of subjects having an immune disease or from a plurality of subjects in which an immune disease cannot be detected using standard methods.

In another embodiment of this aspect and all other aspects described herein, the reference value is obtained from the subject at an earlier time point.

In another embodiment of this aspect and all other aspects described herein, the earlier time point is prior to the onset of symptoms associated with the immune disease.

Another aspect provided herein relates to an assay comprising: a. contacting a biological sample obtained from a subject with a detectable binding agent specific for NAD+; b. optionally washing the sample to remove unbound binding agent, c. measuring the intensity of the signal from the bound, detectable binding agent, d. comparing the measured intensity of the signal with a reference value and if the measured intensity is reduced relative to the reference value, e. identifying the subject as having or having an increased probability of having an immune disease.

In one embodiment of this aspect and all other aspects described herein, the binding agent is an antibody.

In another embodiment of this aspect and all other aspects described herein, the immune disease is selected from the group consisting of Type 1 diabetes, allergy, asthma, eczema, systemic lupus erythematosus, rheumatoid arthritis, transplantation, inflammatory bowel disease, cancer, multiple sclerosis and sepsis. In one embodiment of this aspect and all other aspects described herein, the immune disease is an atopic disorder (e.g., allergy, food allergy, eczema). In another embodiment of this aspect and all other aspects described herein, the immune disease is chronic inflammation (e.g., chronic obstructive pulmonary disease (COPD)). In one embodiment, the immune disease is graft-versus-host disease.

In another embodiment of this aspect and all other aspects described herein, the assay further comprises a step of treating the subject.

In another embodiment of this aspect and all other aspects described herein, the subject is treated with a composition comprising a therapeutically effective amount of NAD+.

In another embodiment of this aspect and all other aspects described herein, treatment with NAD+ or an analog thereof increases systemic IL-10 cytokine production by at least 10% (e.g., at least 20%, at least 30%, at least 40%, at least 50%, at least 60%, at least 70%, at least 80%, at least 90%, at least 1-fold, at least 2-fold, at least 5-fold, at least 10-fold, at least 100-fold or more than the systemic IL-10 cytokine production in the absence of NAD+ or an analog thereof.

In another embodiment of this aspect and all other aspects described herein, the amount of NAD+ is compared to a reference value.

In another embodiment of this aspect and all other aspects described herein, the reference value is obtained from a plurality of subjects in which an immune disease cannot be detected using standard methods.

In another embodiment of this aspect and all other aspects described herein, the reference value is obtained from the subject at an earlier time point.

In another embodiment of this aspect and all other aspects described herein, the earlier time point is prior to the onset of symptoms associated with the immune disease.

Also provided herein, in another aspect, is a method for diagnosing an immune disease in a subject, the method comprising: (a) measuring the amount of NAD+ in a biological sample obtained from a subject suspected of having an immune disease, and (b) comparing the amount of NAD+ measured in the biological sample to the amount of NAD+ in a reference sample, wherein a decrease in the amount of NAD+ compared to the reference value indicates that the subject has an immune disease.

In one embodiment of this aspect and all other aspects described herein, the method further comprises a step of treating the subject.

In another embodiment of this aspect and all other aspects described herein, the subject is treated with a composition comprising a therapeutically effective amount of NAD+.

In another embodiment of this aspect and all other aspects described herein, the amount of NAD+ is compared to a reference value.

In another embodiment of this aspect and all other aspects described herein, the reference value is obtained from a plurality of subjects in which an immune disease cannot be detected using standard methods.

In another embodiment of this aspect and all other aspects described herein, the reference value is obtained from the subject at an earlier time point.

In another embodiment of this aspect and all other aspects described herein, the earlier time point is prior to the onset of symptoms associated with the immune disease.

Another aspect provided herein relates to a composition comprising a therapeutically effective amount of NAD+ for use in the treatment of an immune disease in a subject (e.g., asthma, type 1 diabetes). Alternatively, another aspect provided herein relates to a composition comprising a therapeutically effective amount of an analog of NAD+ (see e.g., The Pyridine Nucleotide Coenzymes, edited by Everse et al., Academic Press, New York, 1982) for use in the treatment of an immune disease in a subject.

In one embodiment of this aspect and all other aspects described herein, the NAD+ analog is: P1-N6-(4-azidophenylethyl)adenosine-P2-[4-(3-azidopyridinio)butyl]diphosphate, P1, P2-(5′-beta-nicotinamideribofuranosyl-3″-adenosyl)-diphosphate (N3″AD+), 3-acetylpyridine-NAD+, βTAD (thiazole-4-carboxamide adenine dinucleotide), N^{o}-[(6-aminohexyl)carbamoylmethyl]-NAD+, 3-acetylpyridine-adenine-dinculeotide, N-4-azido-2-nitrophenyl-4-aminobutyryl-3[prime]-NAD+, or the like.

### BRIEF DESCRIPTION OF THE FIGURES

FIGS. 1A-1C demonstrate that NAD⁺ promotes IL-10 cytokine production in mouse and human CD4⁺ T helper cells under Th1 polarizing conditions. CD4⁺ naïve T cells were isolated from spleens of DBA mice or human PBMCs and cultured in presence of α-CD3, α-CD28, IL-2 with increasing concentrations of NAD⁺ and in presence of Th1 polarizing conditions (20 ng/ml of recombinant IL-12 and 10 μg/ml of anti-IL-4). Mouse CD4+ T cells were cultured for 96 hrs and IFN-γ, IL-10 and IL-17A cytokine production was measured by ELISA (FIG. 1A) and frequencies of IFNγ⁺, IL-10⁻ and IL-17A⁺ cells were assessed by flow cytometry (FIG. 1B). Human CD4+ T cells were cultured for 72 hours and frequencies of IFNγ+, IL-10+ and IFNγ+/IL-10+ cells were assessed by flow cytometry (n=6 per group, representative plots shown in FIG. 1C). *, p<0.05; **, p<0.01, ***, p<0.001; NS, not significant.

FIGS. 2A-2B demonstrate that NAD⁺ skews IL-4⁺/IL-10⁺ producing Th2 cells towards IL-4⁺/IL-17A⁺ producing cells under Th2 polarizing conditions. CD4⁺ naïve T cells were isolated from spleens of DBA mice and were cultured in presence of α-CD3, α-CD28, IL-2 with increasing concentrations of NAD⁺ and in presence of Th2 conditions (20 ng/ml of recombinant IL-4, 10 μg/ml of anti-IFNγ and 10 μg/ml of anti-IL-12). After 96 hrs, IL-4, IL-10 and IL-17A cytokine production was measured by ELISA (FIG. 2A) and frequencies of IL-4⁺, IL-10⁺ and IL-17A⁺ cells were assessed by flow cytometry (FIG. 2B, n=6 per group, representative plots shown). **, p<0.05; ***, p<0.01.

FIGS. 3A-3B demonstrate that NAD⁺ converts naïve CD4⁻ T cells into Th17 cells in iTreg polarizing conditions. CD4^{|} naïve T cells were isolated from spleens of DBA mice and were cultured in presence of α-CD3, α-CD28, IL-2 with increasing concentrations of NAD^{|} and in presence of iTreg polarizing conditions (10 ng/ml of recombinant TGFβ, 10 μg/ml of anti-IL-6, 10 μg/ml of anti-IFNγ, 10 μg/ml of anti-IL-12 and 10 μg/ml of anti-IL4). After 96 hrs IL-10, TGFβ and IL-17A cytokine production was measured by ELISA (FIG. 3A) and frequencies of IL-10⁺, TGFβ⁺ and IL-17A⁺ cells were assessed by flow cytometry (FIG. 3B, n=6 per group, representative plots shown). **, p<0.05; ***, p<0.01.

FIGS. 4A-4C demonstrate that NAD⁺ prevents from EAE and enhances a systemic IL-10 cytokine production by CD4⁺ IFNγ-producing cells. (FIG. 4A) Behavioral scores of EAE in C57BL/6 mice treated or not daily with intraperitoneal injection of 40 mg of NAD⁺ (n=8-11 per group). (FIG. 4B) CD4⁺ T cells were isolated from spleens 18 days after EAE induction and CD4⁺ frequencies of CD25⁺/Foxp3⁺, IL-17A⁺/IL-23R⁺ and IFNγ⁺/IL-10⁺ cells were analyzed by flow cytometry. (FIG. 4C) Number of lymphocytes in the blood and in the spleen of mice treated during 4 days with NAD⁺ or a placebo solution (PBS). (n=6-8 per group, representative plots shown). p<0.05; **, p<0.01; ***, p<0.001, N.S., not significant.

FIGS. 5A-5H demonstrate that NAD⁺ prevents from demyelination and axon loss. Spinal cord was isolated from three groups of mice: (a) mice that were treated daily with a placebo solution (PBS), (b) mice that were treated daily with NAD+, and (c) mice that were treated with NAD+ 15 days after MOG immunization (when mice developed hind limb paralysis). FIGS. 5A-5C: paraffin sections were stained with luxol fast blue (LFB) to assess demyelination, H&E for lymphocyte infiltration, myelin basic protein (MBP) to assess demyelination, neurofilament 200 (NF) for axon loss and DAPI to label nuclei. Merged images of MBP, NF and DAPI are also shown. The boxed regions in FIGS. 5A-5C for LFB and H&E stainings have been magnified in FIGS. 5D-5F, respectively. The white starts in FIGS. 5A-5C for MBP, NF and DAPI and merged images depict regions that are shown in higher magnification in FIGS. 5D-5F. Scale bars: a, 100 μm (applies to FIGS. 5A-5C), d, 50 μm (applies to FIGS. 5D-5F). FIGS. 5G-5H Quantification of myelination and neurofilament positive areas showing a significant protection and recovery of myelin (FIG. 5G) and neurofilaments (FIG. 5H) following daily NAD+ treatment and administration 15 days after MOG immunization. Error bars indicate SD. (n=6 per group). ***, p<0.001

FIGS. 6A-6B demonstrate that NAD⁺ prevents from EAE and regulates CD4⁺ T cell differentiation through Tph-1 and CA3. (FIG. 6A) Behavioral scores of EAE in C57BL/6 mice treated or not daily with intraperitoneal injections of 40 mg of NAD⁺. In addition, mice were treated with p-chlorophenylalanine (Tph-1 inhibitor) (n=5 per group). (FIG. 6B) CD4⁺ T cells were isolated from spleens 13 days after EAE induction and CD4⁺ frequencies of CD25⁺/Foxp3⁺, IL-17A⁺/IL-23R⁺ and IFNγ⁺/IL-10⁺ cells were analyzed by flow cytometry. *, p<0.05; **, p<0.01; ***, p<0.001.

FIGS. 7A-7G demonstrate that NAD^{|} regulates T cell differentiation in Th0 but not in Th17 polarizing conditions. CD4⁺ naïve T cells were isolated from spleen of DBA mice and were cultured in presence of α-CD3, α-CD28, IL-2 and increasing NAD⁺ concentrations. After 96 hrs (FIG. 7A) IFNγ, IL-4, IL-17A, TNFα, IL-10, IL-6, and TGFβ cytokine production was measured by ELISA and (FIG. 7B) frequencies of IFNγ⁻, IL-4⁺⁺ and IL-17A⁺ cells were assessed by flow cytometry. (FIG. 7C) CD4⁻ naïve T cells were isolated from spleen of DBA mice were cultured in presence of α-CD3, α-CD28, IL-2 with increasing NAD⁻ concentrations and in presence of Th1, polarizing conditions. After 96 hrs cytokine production of TNFα and IL-6 cytokine production was measured by ELISA. (FIG. 7D) CD4^{|} naïve T cells were isolated from spleen of DBA mice were cultured in presence of α-CD3, α-CD28, IL-2 with increasing NAD^{|} concentrations and in presence of Th2, polarizing conditions. After 96 hrs cytokine production of IL-6 and TNFα cytokine production was measured by ELISA. (FIGS. 7E-7F) CD4⁺ naïve T cells were isolated from spleen of DBA mice were cultured in presence of α-CD3, α-CD28, IL-2 with increasing NAD⁺ concentrations and in presence of Th1, polarizing conditions. After 96 hrs (FIG. 7E) IL-17A, IFNγ, IL-4, IL-10 and TNFα cytokine production was measured by ELISA and (FIG. 7F) frequencies of IFNγ^{|}, IL-4^{|} and IL-17A⁺ cells were assessed by flow cytometry. (FIG. 7G) CD4⁺ naïve T cells were isolated from spleen of DBA mice were cultured in presence of α-CD3, α-CD28, IL-2 with increasing NAD⁺ concentrations and in presence of iTreg, polarizing conditions. After 96 hrs cytokine production of TNFα and IL-6 cytokine production was measured by ELISA. (n=6 per group, representative plots shown).n.d.; not determined. **, p<0.05; ***, p<0.01; n.d., not detected.

FIGS. 8A-8B demonstrate that ectopic expression of NFIL3 in CD4+ T cells attenuates the gut pathology in adoptive transferred colitis. FIG. 8A. Naïve CD4+ T cells from C57BL/6 mice were transduced with NFIL3-expressing retrovirus (NFIL3) or control empty retrovirus (GFP). Cells were i.p. injected into Rag1 −/− recipient mice to induce gut inflammation. Wasting disease was monitored for 10 weeks after transfer. Statistics is based on the combination of total animals from two independent experiments. Data are shown as mean ± SEM. Mann Whiteny test two-tailed P=0.0064. FIG. 8B. Hematoxylin and eosin staining of small intestine tissue sections 10 weeks after adoptive transfer.

FIG. 9 depicts a model showing that NAD⁺ acts a master regulator of CD4⁺ T helper cell differentiation. After TCR engagement NAD⁺ promotes naïve CD4⁺ T cell into Th1 but not Th2 type cells. NAD⁺ promotes the conversion of Th1-IFNγ producing cells into regulatory type 1 cells that co-produce IFNγ and IL-10 cytokines with immunosuppressive properties. NAD^{|} drives the switch from classical IL-4/IL-10 producing Th2 effector cells toward IL-4/IL-17A producing Th2 cells. NAD⁺ does not affect Th17 cytokine conditions but drives the switch of iTreg in polarizing conditions into nonpathogenic Th17 cells.

FIG. 10 demonstrates that NAD⁺ promotes conversion of CD4^{|}CD25⁺FoxP3⁺ nTregs into IL-17A producing cells. CD4⁺CD25⁻ nTregs were cultured in presence of α-CD3, α-CD28 and IL-2 with increasing concentrations of NAD⁺ and frequency of CD4⁺FoxP3⁺IL-17A⁺ cells was assessed in a dose- and time-dependent manner (n=12 per group, representative plots shown). *, p<0.05; **, p<0.01.

FIGS. 11A-11B demonstrate that NAD⁺ Converts nTregs into Th17 cells specifically. CD4⁺CD25⁺ nTregs were cultured in presence of α-CD3, α-CD28 and IL-2 with increasing concentrations of NAD⁺ and after 96 hrs (FIG. 11A) mRNA levels of nTregs (TGF-β, IL-10), Th1 (IFN-γ) Th2 s (IL-4, IL-6, IL-10) and Th17 (IL-17A) cytokines were measured by real-time PCR (n=8 per group); (FIG. 11B) and protein levels were quantified by ELISA (n=8 per group). *, p<0.05; **, p<0.01; ***, p<0.001.

FIGS. 12A-12C demonstrate that NAD⁺ promotes signals nTreg conversion into to Th17 cells through the transcription factors STAT3 and RORγt. CD4⁺CD25⁺ nTregs were cultured in presence of α-CD3, α-CD28, IL-2 with increasing concentrations of NAD⁺ and after (FIG. 12A) 24 hrs or (FIG. 12B) 48 hrs of culture mRNA levels of Tbet (Tbx21), GATA3, STAT3, STAT5, RORγt and FoxP3 were detected measured by real-time PCR (n=6 per group). (FIG. 12C) CD4⁺CD25⁺ nTregs isolated from STAT3^{-/-} mice were cultured for 96 hrs in presence of α-CD3, α-CD28 IL-2 with or without NAD⁺ and frequencies of CD4⁺IL-17A⁻FoxP3⁻ cells and IL-17A protein levels were determined by flow cytometry and ELISA (n=6 per group). *, p<0.05; **, p<0.01; ***, p<0.001.

FIGS. 13A-13B demonstrate that NAD⁺ promotes conversion of human nTregs into IL-17A producing cells. nTregs were isolated from human PBMCs and cultured in presence of α-CD3, α-CD28, IL-2 and increasing NAD⁺ concentrations. After 96 hrs of culture frequencies of (FIG. 13A) Foxp3⁺ and IL-17A producing cells among CD4⁺CD25⁺ was assessed (experiments were performed in triplicate for each condition, representative plots shown). (FIG. 13B) IL-17A and IL-17F cytokine secretion was measured by ELISA (experiments were performed in triplicate for each condition). *, p<0.05; **, p<0.01; ***, p<0.001.

FIGS. 14A-14C demonstrate that NAD⁺ signals through P2RX4 and P2RX7 receptors. CD4⁺CD25⁺ nTregs were cultured in presence of α-CD3, α-CD28 and IL-2 with increasing concentrations of NAD⁺ and after 24 hrs (FIG. 14A) mRNA levels for P2RX4, P2RX7, P2RY1, P2RY2, and P2RY4 were measured by real-time PCR (n=6 per group) and (FIG. 14B) Freshly isolated T cells were cultured for 24 hrs in the presence of vehicle alone (right column) or 50 μM NAD (left column) Cells were collected and stained at 4C for either P2RX4 (top row) or P2RX7 (bottom row) without prior fixation or permeabilization. Stacks of 20, 2-channel images were acquired in each condition and the resulting stacks were deconvolved and reconstituted for further analysis. The results show that treatment of T cells with NAD increased the cell surface expression levels of both receptors and in the case P2RX4, NAD promotes capping-like distribution pattern. (FIG. 14C) nTregs were cultured as described above with or without MRS 2279 (a selective antagonist of P2RY1), 5-BDBD (a selective antagonist of P2RX4) or A 804598 (a selective antagonist of P2RX7) and IL-17A secretion was measured by ELISA (n=6 per group). *, p<0.05; **, p<0.01; ***, p<0.001.

FIGS. 15A-15C demonstrate that NAD^{|} promotes skin allograft survival through systemic increase in IL-10. Fully MHC-mismatched B6 tail skin allografts were transplanted onto DBA/2 mice that received daily doses of NAD⁺ (250 μM) or control solution (PBS). (FIG. 15A) skin graft survival was monitored (n=6 per group) and (FIG. 15B) CD4⁺ T cells were isolated from spleens 8 days after transplantation and frequencies of IL-10⁺, IL-17A⁺, and IFNγ⁺ cells were analyzed by flow cytometry (n=6 per group, representative plots shown). (FIG. 15C) Fully MHC-mismatched DBA tail skin allografts were transplanted onto IL-10^{-/-} (C57BL/6 background) and wild type (WT) mice that received daily doses of NAD⁺ (250 μM) or control solution (PBS) and skin graft survival onto IL-10−/− and WT mice was monitored (n=6 per group). *, p<0.05; **, p<0.01; ***, p<0.001.

FIGS. 16A-16C demonstrate that NAD⁺ induces nTreg apoptosis and loss of FoxP3 expression in vitro. (FIG. 16A) Purities of CD4⁺CD25⁺ nTregs after isolation of spleen from DBA mice were >98%, containing no contaminating CD11c⁺ cells (plots shown are representative for three independent experiments). (FIG. 16B) Frequencies of nTregs that were cultured in presence of α-CD3, α-CD28, IL-2 and increasing concentrations of NAD⁺ after 24 hrs, 48 hrs and 96 hrs (n=6 per group, representative plots shown). (FIG. 16C) Percentages of Annexin V⁺ cells among CD4⁺CD25⁺FoxP3⁺ nTregs after 24 hrs of culture in presence of α-CD3, α-CD28, IL-2 and increasing concentrations of NAD⁺ (n=6 per group, representative plots shown). *, p<0.05; **, p<0.01; ***, p<0.001.

FIGS. 17A-17B demonstrate that nTregs converted into IL-17A producing cells remain resistant to apoptosis and proliferate in presence of NAD⁺ without exogenous IL-23 cytokine. CD4⁺CD25⁺FoxP3⁺ cells were cultured in presence of α-CD3, α-CD28, IL-2 with increasing concentrations of NAD⁺ and (FIG. 17A) apoptosis of CD4⁺CD25⁻FoxP3⁺ IL-17A⁺ cells was assessed with Annexin V after 48 hrs and 96 hrs in a dose-dependent manner (n=6 per group, representative plots shown). (FIG. 17B) proliferation was assessed with CFSE in a dose-dependent manner (n=6 per group, representative plots shown).

FIG. 18 demonstrates that NAD^{|} promotes allograft survival. Picture of skin grafts from day 6 to day 13 after transplantation in mice treated with NAD⁺ or with a placebo solution. Fully MHC-mismatched tail skin allografts from C57BL/6 mice were transplanted onto DBA/2 recipient mice that received control solution (PBS) or daily doses of NAD⁺ (10 mg). Fully MHC-mismatched DBA tail skin allografts were transplanted onto IL-10^{-/-} mice (C57BL/6 background) that were treated daily with NAD⁺ (10 mg). Pictures of skin grafts were taken daily from day 6 to day 13. For IL-10^{-/-} mice pictures of skin grafts were taken daily from day 5 to day 7.

FIG. 19 depicts a model showing that NAD⁺ alone regulates nTreg conversion into Th17 cells and their proliferation. NAD⁺ promotes nTregs conversion into Th17 cells in absence of exogenous TGFβ and IL-6 cytokines after TCR engagement via purinergic receptors P2RX4 and P2RX7 and the transcription factors STAT3 and RORγt. Th17 cells differentiate in presence of their inhibitory cytokine IL-2 and proliferate in absence of exogenous IL-23 cytokine. In addition, NAD⁺ promotes in vivo a robust systemic IL-10 cytokine response through CD4⁺ T helper cells.

FIGS. 20A-20C depicts data from an experiment where allergy was induced in mice (C57B1/6) with intraperitoneal injections of OVA at day 1, 7, 14 and 21. Mice were treated with OVA or a placebo solution (PBS). NAD+ reduces specifically the frequencies of effector T cells and mature B cells that are induced by the antigen (e.g., Ovalbumin).Thus NAD+ can also reduce inflammatory responses by targeting specifically activated T cells (but not naive T cells) and mature B cells. NAD+ can therefore be used in chronic inflammation and allergy such as chronic obstructive pulmonary disease and atopic disorders. FIG. 20A shows the percentage of CD27+ CD19+ B cells. FIG. 20B shows the percentage of CD44+CD44^{low}CD62L^{high} naïve T cells. FIG. 20C shows the percentage of CD44+CD44^{low}CD62L^{high} central memory T cells.

FIG. 21 shows the effects of NAD+ treatment in a mouse model of allergy. The top panel of the figure is a schematic depicting the experimental protocol. The middle panel shows data relating to cell counts and the bottom panel indicates the number of CD4+CD44^{high}CD62L^{high} cells detected in the presence of ovalbumin or ovalbumin/NAD+.

FIG. 22 is a graph depicting blood glucose levels in a mouse model of Type 1 diabetes over time. Each of the three mice were treated with NAD+ and show a marked decrease in blood glucose levels upon NAD+ treatment.

### DETAILED DESCRIPTION

The methods and assays described herein are based, in part, on the discovery that nicotinamide adenine dinucleotide (NAD+), can activate CD4+ helper T cells, thereby modulating immune responses. Provided herein are methods for treating or preventing an immune disease in a subject that comprise administering a therapeutically effective amount of NAD+ or an analog thereof. Also provided herein are methods and assays for diagnosing an immune disease in a subject by measuring the level of NAD+ in a biological sample obtained from the subject.

### Definitions

As used herein, the terms “treat” “treatment” “treating,” or “amelioration” refer to therapeutic treatments, wherein the object is to reverse, alleviate, ameliorate, inhibit, slow down or stop the progression or severity of a condition associated with, a disease or disorder. The term “treating” includes reducing or alleviating at least one adverse effect or symptom of a condition, disease or disorder associated with an immune disease such as asthma, eczema, systemic lupus erythematosus, rheumatoid arthritis, transplantation (e.g., allograft rejection), inflammatory bowel disease, cancer, multiple sclerosis, and sepsis, among others. Treatment is generally “effective” if one or more symptoms or clinical markers are reduced. Alternatively, treatment is “effective” if the progression of a disease is reduced or halted. That is, “treatment” includes not just the improvement of symptoms or markers, but can also include a cessation or at least slowing of progress or worsening of symptoms that would be expected in absence of treatment. Beneficial or desired clinical results include, but are not limited to, alleviation of one or more symptom(s) of an immune disease, diminishment of extent of the immune disease, stabilized (i.e. , not worsening) state of the immune disease, delay or slowing of progression of the disease, amelioration or palliation of the immune disease state, and remission (whether partial or total), whether detectable or undetectable. The term “treatment” of a disease also includes providing relief from the symptoms or side-effects of the disease (including palliative treatment).

In one embodiment, as used herein, the term “prevention” or “preventing” when used in the context of a subject refers to stopping, hindering, and/or slowing down the development of an immune disease and symptoms associated with the immune disease.

As used herein, the term “therapeutically effective amount” means that amount necessary, at least partly, to attain the desired effect, or to delay the onset of, inhibit the progression of, or halt altogether, the onset or progression of the particular disease or disorder being treated (e.g., an immune disease). Such amounts will depend, of course, on the particular condition being treated, the severity of the condition and individual patient parameters including age, physical condition, size, weight and concurrent treatment. These factors are well known to those of ordinary skill in the art and can be addressed with no more than routine experimentation. In some embodiments, a maximum dose of NAD+ or another agent is used, that is, the highest safe dose according to sound medical judgment. It will be understood by those of ordinary skill in the art, however, that a lower dose or tolerable dose can be administered for medical reasons, psychological reasons or for virtually any other reason.

In one embodiment, a therapeutically effective amount of a pharmaceutical formulation, or a composition described herein for a method of treating an immune disease is an amount of sufficient to reduce the level of at least one symptom of an immune disease (e.g., pain, inflammation, etc.) as compared to the level in the absence of the compound, the combination of compounds, the pharmaceutical composition/formulation or the composition. In other embodiments, the amount of the composition administered is preferably safe and sufficient to treat, delay the development of an immune disease, and/or delay onset of the immune disease. In some embodiments, the amount can thus cure or result in amelioration of the symptoms of an immune disease, slow the course of the disease, slow or inhibit a symptom of the disease, or slow or inhibit the establishment or development of secondary symptoms of the immune disease. For example, an effective amount of a composition described herein inhibits further pain and/or inflammation associated with an immune disease, cause a reduction in or even completely inhibit pain and/or inflammation associated with an immune disease, even initiate complete regression of the immune disease, and reduce clinical symptoms associated with the immune disease. In one embodiment, an effective amount for treating or ameliorating a disorder, disease, or medical condition is an amount sufficient to result in a reduction or complete removal of the symptoms of the disorder, disease, or medical condition. The effective amount of a given therapeutic agent will vary with factors such as the nature of the agent, the route of administration, the size and species of the animal to receive the therapeutic agent, and the purpose of the administration. Thus, it is not possible or prudent to specify an exact “therapeutically effective amount.” However, for any given case, an appropriate “effective amount” can be determined by a skilled artisan according to established methods in the art using only routine experimentation.

In one embodiment, a therapeutically effective amount of NAD+ is the amount that upon administration to a subject increases systemic IL-10 cytokine production by at least 10%.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99% , or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

As used herein, the term “reference value” refers to a reference value, or range of values, obtained for NAD+ from e.g., at least one subject determined to lack a detectable immune disorder. The reference value or range of values can be obtained from a plurality of subjects in a population substantially free of an immune disorder (i.e. , is not detectable by typical clinical means) or alternatively from a plurality of subjects in a population having an immune disease. The reference sample can be stored as a value(s) on a computer or PDA device to permit comparison with a value obtained from a subject using the methods described herein. The reference sample can also be obtained from the same subject e.g., at an earlier time point prior to onset of the immune disease or symptoms thereof using clinical tests known to those of skill in the art. One of skill in the art can determine an appropriate reference sample for use with the methods described herein. In one embodiment, the reference is obtained from a subject or plurality of subjects having, or diagnosed with having, an immune disease such as Type 1 diabetes, allergy, asthma, eczema, systemic lupus erythematosus, rheumatoid arthritis, allograft rejection, bowel disease, cancer, multiple sclerosis, sepsis, and autoimmune disease, among others.

As used herein, the terms “biological sample” refers to a fluid sample, a cell sample, a tissue sample or an organ sample obtained from a subject or patient. Biological samples include, but are not limited to, tissue biopsies, tumor biopsies, scrapes (e.g. buccal scrapes), whole blood, plasma, serum, urine, saliva, cell culture, intestinal lavage, cerebrospinal fluid, circulating tumor cells, and the like. Samples can include frozen or paraffin-embedded tissue. The term “sample” includes any material derived by processing such a sample. Derived samples may, for example, include nucleic acids or proteins extracted from the sample or obtained by subjecting the sample to techniques such as amplification or reverse transcription of mRNA, isolation and/or purification of certain components, etc.

The terms “increased” ,“increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, at least about a 20-fold increase, at least about a 50-fold increase, at least about a 100-fold increase, at least about a 1000-fold increase or more as compared to a reference level.

The term “statistically significant” or “significantly” refers to statistical significance and generally means a two standard deviation (2SD) below normal, or lower, e.g., level of NAD+. The term refers to statistical evidence that there is a difference. It is defined as the probability of making a decision to reject the null hypothesis when the null hypothesis is actually true. The decision is often made using the p-value.

As used herein, the terms “free from detectable immune disease” and “substantially free of an immune disease” are used interchangeably and refer to subjects that do not exhibit any clinically detectable signs of an immune disease using routine clinical methods known to those skilled in the art (e.g., routine visual inspection by a health care professional; imaging such as blood screening, ultrasound, CAT scan, endoscopy, CT scan, MRI; palpation; mammogram; routine biopsy, etc).

As used herein, the term “plurality” refers to at least two subjects in a population used to define a reference level of NAD+, for example, at least 3, at least 4, at least 5, at least 6, at least 7, at least 8, at least 9, at least 10, at least 15, at least 20, at least 25, at least 30, at least 35, at least 40, at least 45, at least 50, at least 60, at least 70, at least 80, at least 90, at least 100, at least 125, at least 150, at least 175, at least 200, at least 300, at least 400, at least 500, at least 600, at least 700, at least 800, at least 900, at least 1000, at least 1500, at least 2000, at least 5000, at least 10⁴, at least 10⁵, at least 10⁶, or more subjects in a population.

The term “pharmaceutically acceptable” refers to compounds and compositions which may be administered to mammals without undue toxicity. The term “pharmaceutically acceptable carriers” excludes tissue culture medium. Exemplary pharmaceutically acceptable salts include but are not limited to mineral acid salts such as hydrochlorides, hydrobromides, phosphates, sulfates, and the like, and the salts of organic acids such as acetates, propionates, malonates, benzoates, and the like.

The term “NAD-related compounds” includes, quinolinic acid; quinolinic acid ribonucleotide; nicotinamide; nicotinic acid; nicotinic acid ribonucleotide; nicotinic acid ribonucleotide, reduced form; nicotinamide ribonucleotide; nicotinamide ribonucleotide, reduced form; nicotinic acid adenine dinucleotide; nicotinic acid adenine dinucleotide, reduced form; nicotinamide adenine dinucleotide (NAD); nicotinamide adenine dinucleotide phosphate (NADP); nicotinamide adenine dinucleotide, reduced form (NADH); and nicotinamide adenine dinucleotide phosphate, reduced form (NADPH) and pharmaceutically acceptable salts thereof. All of these chemicals are commercially available or are generally known. Preferably the NAD-related compound is nicotinamide or nicotinic acid, more preferably the NAD-related compound is nicotinamide. In any event, the NAD-related compounds other than NAD, NADH, NADPH OR NADP, must be capable of entering the enzymatic pathways available in the mammalian body resulting in the production of NAD or NADH.

As used herein, the term “comprising” means that other elements can also be present in addition to the defined elements presented. The use of “comprising” indicates inclusion rather than limitation.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

Further, unless otherwise required by context, singular terms shall include pluralities and plural terms shall include the singular.

Other than in the operating examples, or where otherwise indicated, all numbers expressing quantities of ingredients or reaction conditions used herein should be understood as modified in all instances by the term “about.” The term “about” when used in connection with percentages can mean ±1%.

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

### Immune Diseases

Essentially any immune disease or disorder can be diagnosed or treated using the methods and compositions described herein. The term “immune disease or disorder” also includes both acute and chronic inflammation.

In some embodiments, the term “immune disease or disorder” refers to diseases and conditions associated with inflammation which include but are not limited to: (1) inflammatory or allergic diseases such as systemic anaphylaxis or hypersensitivity responses, drug allergies, insect sting allergies; inflammatory bowel diseases, such as Crohn's disease, ulcerative colitis, ileitis and enteritis; vaginitis; psoriasis and inflammatory dermatoses such as dermatitis, eczema, atopic dermatitis, allergic contact dermatitis, urticaria; vasculitis; spondyloarthropathies; scleroderma; respiratory allergic diseases such as asthma, allergic rhinitis, hypersensitivity lung diseases, and the like, Type 1 diabetes, (2) autoimmune diseases, such as arthritis (rheumatoid and psoriatic), osteoarthritis, multiple sclerosis, systemic lupus erythematosus, Type 1 diabetes, diabetes mellitus, glomerulonephritis, and the like, (3) graft rejection (including allograft rejection and graft-v-host disease), and (4) other diseases in which undesired inflammatory responses are to be inhibited (e.g., myositis, inflammatory CNS disorders such as stroke and closed-head injuries, neurodegenerative diseases, Alzheimer's disease, encephalitis, meningitis, osteoporosis, gout, hepatitis, nephritis, sepsis, sarcoidosis, conjunctivitis, otitis, chronic obstructive pulmonary disease, sinusitis and Bechet's syndrome).

In other embodiments, the term “immune disease or disorder” refers to a state of acute or chronic inflammation. An acute inflammatory response is an immediate response by the immune system to a harmful agent. The response includes vascular dilatation, endothelial and neutrophil activation. An acute inflammatory response will either resolve or develop into chronic inflammation.

Chronic inflammation is an inflammatory response of prolonged duration, weeks, months, or even indefinitely, whose extended time course is provoked by the persistence of the causative stimulus to inflammation within the tissue or the development of an autoimmune disorder. The inflammatory process inevitably causes tissue damage. The exact nature, extent and time course of chronic inflammation is variable, and depends on a balance between the causative agent and the attempts of the body to remove it. Agents producing chronic inflammation include, but are not limited to: infectious organisms that can avoid or resist host defenses and so persist in the tissue for a prolonged period; infectious organisms that are not innately resistant but persist in damaged regions where they are protected from host defenses; irritant nonliving foreign material that cannot be removed by enzymatic breakdown or phagocytosis; or where the stimuli is a “normal” tissue component, causing an auto-immune disease. There is a vast array of diseases exhibiting a chronic inflammatory component. These include but are not limited to: inflammatory joint diseases (e.g., rheumatoid arthritis, osteoarthritis, polyarthritis and gout), chronic inflammatory connective tissue diseases (e.g., systemic lupus erythematosus, scleroderma, Sjorgen's syndrome, poly- and dermatomyositis, vasculitis, mixed connective tissue disease (MCTD), tendonitis, synovitis, bacterial endocarditis, osteomyelitis and psoriasis); chronic inflammatory lung diseases (e.g., chronic respiratory disease, pneumonia, fibrosing alveolitis, chronic bronchitis, bronchiectasis, emphysema, silicosis and other pneumoconiosis and tuberculosis); chronic inflammatory bowel and gastro-intestinal tract inflammatory diseases (e.g., ulcerative colitis and Crohn's disease); chronic neural inflammatory diseases (e.g., chronic inflammatory demyelinating polyradiculoneuropathy, chronic inflammatory demyelinating polyneuropathy, multiple sclerosis, Guillan-Barre Syndrome and myasthenia gravis); other inflammatory diseases (e.g., mastitis, laminitis, laryngitis, chronic cholecystitis, Hashimoto's thyroiditis, inflammatory breast disease); chronic inflammation caused by an implanted foreign body in a wound; and including chronic inflammatory renal diseases including crescentic glomerulonephritis, lupus nephritis, ANCA-associated glomerulonephritis, focal and segmental necrotizing glomerulonephritis, IgA nephropathy, membranoproliferative glomerulonephritis, cryoglobulinaemia and tubulointerstitial nephritis. Diabetic nephropathy may also have a chronic inflammatory component and chronic inflammatory responses are involved in the rejection of transplanted organs. Other non-limiting examples of diseases with symptoms of chronic inflammation include obesity, diabetes, inflammatory bowel diseases such as Crohn's disease and ulcerative colitis, psoriasis, sarcoidosis, atherosclerosis including plaque rupture, Sjogrens disease, acne rosacea, syphilis, chemical burns, bacterial ulcers, fungal ulcers, Behcet's syndrome, Stevens-Johnson's disease, Mycobacteria infections, Herpes simplex infections, Herpes zoster infections, protozoan infections, Mooren's ulcer, leprosy, Wegener's sarcoidosis, pemphigoid, lupus, systemic lupus erythematosis, polyarteritis, Lyme's disease, Bartonelosis, tuberculosis, histoplasmosis and toxoplasmosis.

Essentially any disease or disorder characterized by, caused by, resulting from, or becoming affected by inflammation can be treated with the methods and compositions described herein.

### Obtaining a Biological Sample

A biological sample can be obtained from essentially any tissue including but not limited to, blood, plasma, serum, circulating cells, circulating tumor cells, brain, liver, lung, gut, stomach, fat, muscle, spleen, testes, uterus, urinary tract, bladder, prostate, esophagus, ovary, skin, endocrine organ, pancreas, and bone, etc. In one embodiment, a biological sample comprises cells including, but not limited to, epithelial, endothelial, neuronal, adipose, cardiac, skeletal muscle, fibroblast, immune cells, hepatic, splenic, lung, circulating blood cells, reproductive cells, gastrointestinal, renal, bone marrow, and pancreatic cells. In one embodiment, the biological sample is a biopsy from a growth or tumor.

In one embodiment, the biological sample comprises a tissue biopsy, such as, an aspiration biopsy, a brush biopsy, a surface biopsy, a needle biopsy, a punch biopsy, an excision biopsy, an open biopsy, an incision biopsy or an endoscopic biopsy, or a tumor sample. Biological samples can also be biological fluid samples, including but not limited to, urine, blood, serum, platelets, saliva, cerebrospinal fluid, nipple aspirates, circulating tumor cells, and cell lysate (e.g. supernatant of whole cell lysate, microsomal fraction, membrane fraction, exosomes, or cytoplasmic fraction). Samples can be obtained by any method known to one of skill in the art including e.g., needle biopsy, fine needle aspiration, core needle biopsy, vacuum assisted biopsy, open surgical biopsy, among others.

### Reference Value

As used herein, the terms “reference value” and “reference” refer to the level of NAD+, as that term is used herein, in a known sample against which another sample is compared (i.e., obtained from a subject suspected of having an immune disease or disorder). A standard is useful for determining the amount of NAD+ or the relative increase/decrease of NAD+ in a biological sample. A standard serves as a reference level for comparison, such that samples can be normalized to an appropriate standard in order to infer the presence, absence or extent of an immune disorder in a subject.

In one embodiment, a biological standard is obtained at an earlier time point (presumably prior to the onset of an immune disease) from the same individual that is to be tested or treated as described herein. Alternatively, a standard can be from the same individual having been taken at a time after the onset or diagnosis of such an immune disease. In such instances, the standard can provide a measure of the efficacy of treatment.

A standard level can be obtained, for example, from a known biological sample from a different individual (e.g., not the individual being tested) that is substantially free of an immune disease. A known sample can also be obtained by pooling samples from a plurality of individuals to produce a standard over an averaged population, wherein a standard represents an average level of NAD+ among a population of individuals. Thus, the level of NAD+ in a standard obtained in this manner is representative of an average level of this marker in a general population or a diseased population. An individual sample is compared to this population standard by comparing the level of NAD+ from a sample relative to the population standard. Generally, a decrease in the amount of NAD+ over a standard (e.g., obtained from subjects substantially free of an immune disease) will indicate the presence of an immune disease, while an increase in the amount of NAD+ will indicate no immune disease is present. The converse is contemplated in cases where a standard is obtained from a population of subjects having an immune disease. It should be noted that there is often variability among individuals in a population, such that some individuals will have higher levels of NAD+, while other individuals have lower levels of NAD+. However, one skilled in the art can make logical inferences on an individual basis regarding the detection and treatment of the immune disease as described herein.

A standard or series of standards can also be synthesized. A known amount of NAD+ (or a series of known amounts) can be prepared within the typical range for NAD+ that is observed in a general population. This method has an advantage of being able to compare the extent of disease in two individuals in a mixed population. This method can also be useful for subjects who lack a prior sample to act as a standard or for routine follow-up post-diagnosis. This type of method can also allow standardized tests to be performed among several clinics, institutions, or countries etc.

### Detection of NAD+

Nicotinamide adenine dinucleotide (NAD) and its derivative compounds are essential coenzymes in cellular redox reactions in all living organisms. Several lines of evidence have also shown that NAD participates in a number of important signaling pathways in mammalian cells, including poly(ADP-ribosyl)ation in DNA repair (Menissier de Murcia et al., EMBO J., (2003) 22, 2255-2263), mono-ADP-ribosylation in the immune response and G protein-coupled signaling (Corda and Di Girolamo, EMBO J., (2003) 22, 1953-8), and the synthesis of cyclic ADP-ribose and nicotinate adenine dinucleotide phosphate (NAADP) in intracellular calcium signaling (Lee, Annu. Rev. Pharmacol. Toxicol., (2001) 41, 317-345). Recently, it has also been shown that NAD and its derivatives play an important role in transcriptional regulation (Lin and Guarente, Curr. Opin. Cell. Biol., (2003) 15, 241-246).

NAD+ can be detected by any means known in the art. In some embodiments, NAD+ is detected and/or measured using an enzyme linked assay, for example, by reconstituting the NAD biosynthesis pathway in vitro as described in e.g., PCT Publication No. WO2006/041624. In one embodiment, the assay is an enzyme-coupled fluorometric assay that can be used to measure NAD biosynthesis. In one embodiment, the enzyme-coupled reaction measures the fluorescence of NADH detected by a fluorometer following conversion of NAD to NADH by alcohol dehydrogenase.

Quantification of NAD+ and/or NADH can include, for example, a determination of the relative amounts or concentration of NAD+ and/or NADH in the assay mixture. Quantifying NAD+ or NADH can be according to, for example, high performance liquid chromatography of NAD+ or autofluorescence of NADH, respectively.

Alcohol dehydrogenase and ethanol can be present in the reaction mixture employed by the method of identifying compounds that effect NAD biosynthesis. Where alcohol dehydrogenase and ethanol are present, detection or quantification of NADH can include, for example, detecting the fluorescence of the assay mixture and then correlating this fluorescence to the concentration of NADH produced in the assay mixture. Detection of the autofluorescence of NADH can be performed with, for example, a commercially available fluorometer. Alcohol dehydrogenase and ethanol can be present in the various embodiments that include NAD detection, NADH detection, quantification of NAD, quantification of NADH, and determinations of increases or decreases of NAD, NADH, or both.

In another embodiment, NAD+ can be detected and/or measured colorimetrically.

NAD+ can also be measured using an assay kit obtained commercially from e.g., ABCAM, MBL INTERNATIONAL, CAYMAN CHEMICALS, ABNOVA, SIGMA-ALDRICH, AAT BIOQUEST, among others.

In one embodiment, NAD+ is detected as described herein in the Examples section.

### Dosage and Administration

In one aspect, the methods described herein provide a method for an immune disease (e.g., asthma, eczema, systemic lupus erythematosus, rheumatoid arthritis, transplantation, inflammatory bowel disease, cancer, multiple sclerosis and sepsis, among others) in a subject. In one embodiment of this aspect and all other aspects described herein, the immune disease is an atopic disorder (e.g., allergy, food allergy, eczema). In another embodiment of this aspect and all other aspects described herein, the immune disease is chronic inflammation. In one embodiment, the subject can be a mammal. In another embodiment, the mammal can be a human, although the approach is effective with respect to all mammals. The method comprises administering to the subject an effective amount of a pharmaceutical composition comprising NAD+, in a pharmaceutically acceptable carrier. In other embodiments, the methods comprise administering to the subject an effective amount of a pharmaceutical composition comprising an analog of NAD+, in a pharmaceutically acceptable carrier.

The dosage range for the agent depends upon the potency, and includes amounts large enough to produce the desired effect, e.g., immune response modulation. The dosage should not be so large as to cause unacceptable adverse side effects. Generally, the dosage will vary with the type of inhibitor (e.g., an antibody or fragment, small molecule, siRNA, etc.), and with the age, condition, and sex of the patient. The dosage can be determined by one of skill in the art and can also be adjusted by the individual physician in the event of any complication. Typically, the dosage ranges from 0.001 mg/kg body weight to 5 g/kg body weight. In some embodiments, the dosage range is from 0.001 mg/kg body weight to 1 g/kg body weight, from 0.001 mg/kg body weight to 0.5 g/kg body weight, from 0.001 mg/kg body weight to 0.1 g/kg body weight, from 0.001 mg/kg body weight to 50 mg/kg body weight, from 0.001 mg/kg body weight to 25 mg/kg body weight, from 0.001 mg/kg body weight to 10 mg/kg body weight, from 0.001 mg/kg body weight to 5 mg/kg body weight, from 0.001 mg/kg body weight to 1 mg/kg body weight, from 0.001 mg/kg body weight to 0.1 mg/kg body weight, from 0.001 mg/kg body weight to 0.005 mg/kg body weight. Alternatively, in some embodiments the dosage range is from 0.1 g/kg body weight to 5 g/kg body weight, from 0.5 g/kg body weight to 5 g/kg body weight, from 1 g/kg body weight to 5 g/kg body weight, from 1.5 g/kg body weight to 5 g/kg body weight, from 2 g/kg body weight to 5 g/kg body weight, from 2.5 g/kg body weight to 5 g/kg body weight, from 3 g/kg body weight to 5 g/kg body weight, from 3.5 g/kg body weight to 5 g/kg body weight, from 4 g/kg body weight to 5 g/kg body weight, from 4.5 g/kg body weight to 5 g/kg body weight, from 4.8 g/kg body weight to 5 g/kg body weight. In one embodiment, the dose range is from 5 μg/kg body weight to 30 μg/kg body weight. Alternatively, the dose range will be titrated to maintain serum levels between 5 μg/mL and 30 μg/mL.

Administration of the doses recited above can be repeated for a limited period of time. In some embodiments, the doses are given once a day, or multiple times a day, for example but not limited to three times a day. In a preferred embodiment, the doses recited above are administered daily for several weeks or months. The duration of treatment depends upon the subject's clinical progress and responsiveness to therapy. Continuous, relatively low maintenance doses are contemplated after an initial higher therapeutic dose.

A therapeutically effective amount is an amount of an agent that is sufficient to produce a statistically significant, measurable change in immune response (see “Efficacy Measurement” below). Such effective amounts can be gauged in clinical trials as well as animal studies for a given agent.

Agents useful in the methods and compositions described herein can be administered topically, intravenously (by bolus or continuous infusion), orally, by inhalation, intraperitoneally, intramuscularly, subcutaneously, intracavity, and can be delivered by peristaltic means, if desired, or by other means known by those skilled in the art. In one embodiment it is preferred that the agents for the methods described herein are administered directly to a tumor (e.g., during surgery or by direct injection). The agent can be administered systemically, if so desired.

Therapeutic compositions containing at least one agent can be conventionally administered in a unit dose. The term “unit dose” when used in reference to a therapeutic composition refers to physically discrete units suitable as unitary dosage for the subject, each unit containing a predetermined quantity of active material calculated to produce the desired therapeutic effect in association with the required physiologically acceptable diluent, i.e., carrier, or vehicle.

The compositions are administered in a manner compatible with the dosage formulation, and in a therapeutically effective amount. The quantity to be administered and timing depends on the subject to be treated, capacity of the subject's system to utilize the active ingredient, and degree of therapeutic effect desired. An agent can be targeted by means of a targeting moiety, such as e.g., an antibody or targeted liposome technology. In some embodiments, an agent can be targeted to a tissue by using bispecific antibodies, for example produced by chemical linkage of an anti-ligand antibody (Ab) and an Ab directed toward a specific target. To avoid the limitations of chemical conjugates, molecular conjugates of antibodies can be used for production of recombinant bispecific single-chain Abs directing ligands and/or chimeric inhibitors at cell surface molecules. The addition of an antibody to an agent permits the agent to accumulate additively at the desired target site (e.g., tumor or lesion). Antibody-based or non- antibody-based targeting moieties can be employed to deliver a ligand or the inhibitor to a target site. Preferably, a natural binding agent for an unregulated or disease associated antigen is used for this purpose.

Precise amounts of active ingredient required to be administered depend on the judgment of the practitioner and are particular to each individual. However, suitable dosage ranges for systemic application are disclosed herein and depend on the route of administration. Suitable regimes for administration are also variable, but are typified by an initial administration followed by repeated doses at one or more intervals by a subsequent injection or other administration. Alternatively, continuous intravenous infusion sufficient to maintain concentrations in the blood in the ranges specified for in vivo therapies are contemplated.

### Pharmaceutical Compositions

The present invention includes, but is not limited to, therapeutic compositions useful for practicing the therapeutic methods described herein. Therapeutic compositions contain a physiologically tolerable carrier together with an active agent as described herein, dissolved or dispersed therein as an active ingredient. In a preferred embodiment, the therapeutic composition is not immunogenic when administered to a mammal or human patient for therapeutic purposes. As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like. A pharmaceutically acceptable carrier will not promote the raising of an immune response to an agent with which it is admixed, unless so desired. The preparation of a pharmacological composition that contains active ingredients dissolved or dispersed therein is well understood in the art and need not be limited based on formulation. Typically such compositions are prepared as injectable either as liquid solutions or suspensions, however, solid forms suitable for solution, or suspensions, in liquid prior to use can also be prepared. The preparation can also be emulsified or presented as a liposome composition. The active ingredient can be mixed with excipients which are pharmaceutically acceptable and compatible with the active ingredient and in amounts suitable for use in the therapeutic methods described herein. Suitable excipients include, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient. The therapeutic composition of the present invention can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active agent used in the methods described herein that will be effective in the treatment of a particular disorder or condition will depend on the nature of the disorder or condition, and can be determined by standard clinical techniques.

### Efficacy Measurement

The efficacy of a given treatment for an immune disease (e.g., Type 1 diabetes, allergy, asthma, eczema, systemic lupus erythematosus, rheumatoid arthritis, allograft rejection, transplantation, inflammatory bowel disease, cancer, multiple sclerosis and sepsis, among others) can be determined by the skilled clinician. However, a treatment is considered “effective treatment,” as the term is used herein, if any one or all of the signs or symptoms of the immune disease is/are altered in a beneficial manner, other clinically accepted symptoms or markers of disease are improved, or even ameliorated, e.g., by at least 10% following treatment with an agent that comprises NAD+ or an analog thereof. Efficacy can also be measured by a failure of an individual to worsen as assessed by stabilization of the immune disease, hospitalization or need for medical interventions (i.e., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing progression of the immune disease; or (2) relieving the disease, e.g., causing regression of symptoms; and (3) preventing or reducing the likelihood of the development of the immune disease, or preventing secondary diseases/disorders associated with the immune disease (e.g., scarring, tumors, cancer metastasis).

An effective amount for the treatment of a disease means that amount which, when administered to a mammal in need thereof, is sufficient to result in effective treatment as that term is defined herein, for that disease. Efficacy of an agent can be determined by assessing physical indicators of the immune disease, such as e.g., redness, pain, inflammation, lung capacity, size of lesions, tumor growth rate, mobility of subject, etc.

### Systems

Embodiments of the invention also provide for systems (and computer readable media for causing computer systems) to perform a method for diagnosing an immune disease or disorder in a subject, or assessing a subject's risk of developing such a disease or disorder.

Embodiments of the invention can be described through functional modules, which are defined by computer executable instructions recorded on computer readable media and which cause a computer to perform method steps when executed. The modules are segregated by function for the sake of clarity. However, it should be understood that the modules/systems need not correspond to discreet blocks of code and the described functions can be carried out by the execution of various code portions stored on various media and executed at various times. Furthermore, it should be appreciated that the modules may perform other functions, thus the modules are not limited to having any particular functions or set of functions.

The computer readable storage media #30 can be any available tangible media that can be accessed by a computer. Computer readable storage media includes volatile and nonvolatile, removable and non-removable tangible media implemented in any method or technology for storage of information such as computer readable instructions, data structures, program modules or other data. Computer readable storage media includes, but is not limited to, RAM (random access memory), ROM (read only memory), EPROM (erasable programmable read only memory), EEPROM (electrically erasable programmable read only memory), flash memory or other memory technology, CD-ROM (compact disc read only memory), DVDs (digital versatile disks) or other optical storage media, magnetic cassettes, magnetic tape, magnetic disk storage or other magnetic storage media, other types of volatile and non-volatile memory, and any other tangible medium which can be used to store the desired information and which can accessed by a computer including and any suitable combination of the foregoing.

Computer-readable data embodied on one or more computer-readable storage media may define instructions, for example, as part of one or more programs that, as a result of being executed by a computer, instruct the computer to perform one or more of the functions described herein, and/or various embodiments, variations and combinations thereof. Such instructions may be written in any of a plurality of programming languages, for example, Java, J#, Visual Basic, C, C#, C++, Fortran, Pascal, Eiffel, Basic, COBOL assembly language, and the like, or any of a variety of combinations thereof. The computer-readable storage media on which such instructions are embodied may reside on one or more of the components of either of a system, or a computer readable storage medium described herein, may be distributed across one or more of such components.

The computer-readable storage media can be transportable such that the instructions stored thereon can be loaded onto any computer resource to implement the aspects of the present invention(s) discussed herein. In addition, it should be appreciated that the instructions stored on the computer-readable medium, described above, are not limited to instructions embodied as part of an application program running on a host computer. Rather, the instructions can be embodied as any type of computer code (e.g., software or microcode) that can be employed to program a computer to implement aspects of the present invention. The computer executable instructions can be written in a suitable computer language or combination of several languages. Basic computational biology methods are known to those of ordinary skill in the art and are described in, for example, Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2nd ed., 2001).

The functional modules of certain embodiments of the invention(s) include at minimum a determination system #40, a storage device #30, a comparison module #80, and a display module #110. The functional modules can be executed on one, or multiple, computers, or by using one, or multiple, computer networks. The determination system has computer executable instructions to provide e.g., NAD+ concentration information in computer readable form.

The determination system #40, can comprise any system for detecting a signal representing the level of NAD+. Such systems can include colorimetric assays, UV absorbance assays, enzyme cycling assays etc.

The information determined in the determination system can be read by the storage device #30. As used herein the “storage device” is intended to include any suitable computing or processing apparatus or other device configured or adapted for storing data or information. Examples of electronic apparatus suitable for use with the present invention include stand-alone computing apparatus, data telecommunications networks, including local area networks (LAN), wide area networks (WAN), Internet, Intranet, and Extranet, and local and distributed computer processing systems. Storage devices also include, but are not limited to: magnetic storage media, such as floppy discs, hard disc storage media, magnetic tape, optical storage media such as CD-ROM, DVD, electronic storage media such as RAM, ROM, EPROM, EEPROM and the like, general hard disks and hybrids of these categories such as magnetic/optical storage media. The storage device is adapted or configured for having recorded thereon values representing levels of NAD+ information. Such information may be provided in digital form that can be transmitted and read electronically, e.g., via the Internet, on diskette, via USB (universal serial bus) or via any other suitable mode of communication.

As used herein, “stored” refers to a process for encoding information on the storage device. Those skilled in the art can readily adopt any of the presently known methods for recording information on known media to generate manufactures comprising expression information.

In one embodiment the reference data stored in the storage device to be read by the comparison module is e.g., NAD+ data obtained from a population of subjects that are substantially free of immune disease.

The “comparison module” #80 can use a variety of available software programs and formats for the comparison operative to compare sequence information data determined in the determination system to reference samples and/or stored reference data. In one embodiment, the comparison module is configured to use pattern recognition techniques to compare information from one or more entries to one or more reference data patterns. The comparison module can be configured using existing commercially-available or freely-available software for comparing patterns, and may be optimized for particular data comparisons that are conducted. The comparison module provides computer readable information related to the amount of NAD+ present in a biological sample obtained from a subject.

The comparison module, or any other module of the invention, can include an operating system (e.g., UNIX) on which runs a relational database management system, a World Wide Web application, and a World Wide Web server. World Wide Web application includes the executable code necessary for generation of database language statements (e.g., Structured Query Language (SQL) statements). Generally, the executables will include embedded SQL statements. In addition, the World Wide Web application may include a configuration file which contains pointers and addresses to the various software entities that comprise the server as well as the various external and internal databases which must be accessed to service user requests. The Configuration file also directs requests for server resources to the appropriate hardware—as may be necessary should the server be distributed over two or more separate computers. In one embodiment, the World Wide Web server supports a TCP/IP protocol. Local networks such as this are sometimes referred to as “Intranets.” An advantage of such Intranets is that they allow easy communication with public domain databases residing on the World Wide Web (e.g., the GenBank or Swiss Pro World Wide Web site). Thus, in one embodiment of the methods described herein, users can directly access data (via Hypertext links for example) residing on Internet databases using a HTML interface provided by Web browsers and Web servers.

The comparison module provides a computer readable comparison result that can be processed in computer readable form by predefined criteria, or criteria defined by a user, to provide a content based in part on the comparison result that can be stored and output as requested by a user using a display module #110.

The content based on the comparison result, can be data relating to the amount of NAD+ in a biological sample indicating the presence or absence of an immune disease in a subject.

In one embodiment of the invention, the content based on the comparison result is displayed on a computer monitor #120. In one embodiment of the invention, the content based on the comparison result is displayed through printable media #130, #140. The display module can be any suitable device configured to receive from a computer and display computer readable information to a user. Non-limiting examples include, for example, general-purpose computers such as those based on Intel PENTIUM-type processor, Motorola PowerPC, Sun UltraSPARC, Hewlett-Packard PA-RISC processors, any of a variety of processors available from Advanced Micro Devices (AMD) of Sunnyvale, Calif., or any other type of processor, visual display devices such as flat panel displays, cathode ray tubes and the like, as well as computer printers of various types.

In one embodiment, a World Wide Web browser is used for providing a user interface for display of the content based on the comparison result. It should be understood that other modules of the systems described herein can be adapted to have a web browser interface. Through the Web browser, a user may construct requests for retrieving data from the comparison module. Thus, the user will typically point and click to user interface elements such as buttons, pull down menus, scroll bars and the like conventionally employed in graphical user interfaces.

The methods described herein therefore provide for systems (and computer readable media for causing computer systems) to perform methods for diagnosing cancer or assessing risk for developing such a disorder in a subject.

Systems and computer readable media described herein are merely illustrative embodiments of the invention(s) described herein for performing methods of diagnosis in an individual, and are not intended to limit the scope of the invention. Variations of the systems and computer readable media described herein are possible and are intended to fall within the scope of the invention.

The modules of the machine, or those used in the computer readable medium, may assume numerous configurations. For example, function may be provided on a single machine or distributed over multiple machines.

It is understood that the foregoing description and the following examples are illustrative only and are not to be taken as limitations upon the scope of the invention. Various changes and modifications to the disclosed embodiments, which will be apparent to those of skill in the art, may be made without departing from the spirit and scope of the present invention. Further, all patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

All patents and other publications identified are expressly incorporated herein by reference for the purpose of describing and disclosing, for example, the methodologies described in such publications that could be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

### EXAMPLES

### Example 1

### NAD⁺: A Master Regulator of CD4⁺ T Helper Cell Differentiation that Protects from EAE

CD4⁺ T helper lymphocytes play a critical role in the adaptive immune response. For the past quarter century, cytokines have been considered as the major determinants for CD4⁺ T helper cell differentiation. Here, NAD⁺, a natural cofactor found in the human body, is shown to regulate CD4⁺ T cell differentiation and more importantly override the effects of Th1, Th2 and iTreg polarizing conditions. NAD⁻ skewed naïve CD4⁻ T cells towards Th1 cells but not Th2. NAD⁺ switched Th1 cells into regulatory type 1 cells and skewed IL4⁺/IL10⁺ Th2 towards IL-4⁺/IL-17A⁺ producing cells. In iTreg polarizing conditions, NAD⁺ promoted Th17 cell development. In vivo, NAD⁺ reduced Treg cell frequency and enhanced Th17 response but was still able to protect mice from EAE through regulatory type 1 cells by Tph-1 and CA3 activation and by promoting myelin regeneration and preventing axon loss. Thus, as demonstrated herein, NAD⁺ is a master regulator of CD4⁺ T cell differentiation and the immune response and can be used therapeutically in the treatment of autoimmune diseases and beyond.

CD4⁺ helper T (Th) cells play a central role in regulating the adaptive immune response associated with pathogen invasion and numerous diseases including autoimmunity, allergic responses, transplantation as well as tumor immunity¹⁻². TCR activation and stimulation in the periphery in a specific cytokine environment can result in the differentiation of naïve CD4⁺ T cells into distinct lineages of Th cells such as Th1, Th2, Th17 and induced regulatory T cells (iTregs) with distinct functions and non-stochastic cytokine production³. CD4^{|} T cell differentiation was first described in 1986 by Mossman & Coffman showing that CD4^{|} T cells could be divided in two major groups Th1 and Th2⁴⁻⁷. Th1 and Th2 T cell subsets can mainly be distinguished by their cytokine profile, the expression pattern of cell surface molecules and the activation of specific transcription factors. The two other major CD4⁺ T cell subsets, Th17 and iTregs, were characterized recently and were described as distinct lineages from Th1 and Th2 only a decade and a half ago⁸⁻¹⁰.

Naïve CD4⁺ T cell differentiation into Th1 cells requires the transcription factors STAT1, STAT4, Tbx21 and IL-12^{3,11}. Th1 cells produce IFN-γ, IL-2, and TNF-α and are known to enhance clearance of intracellular pathogens^{3,11-12}. In particular conditions, such as a chronic Th1 stimulation or a high dose antigenic stimulation, Th1 cells can also produce IL-13 and IL-10, two cytokines that have been originally described as Th2 cytokines¹³. It was also shown that co-stimulation of human lymphocytes with CD46, a complement receptor, enhances IL-10 production by IFN-γ-producing Th1 effector cells and were termed regulatory type 1 (Tr1) cells because of their immunosuppressive properties¹⁴⁻¹⁶. Th2 cells which play a cardinal feature in parasitic infections produce IL-4, IL-5, IL-6, IL-10 and IL-13 and require the activation of STAT6, and the transcription factor GATA3³. Th17 cells secrete IL-17A, IL-17F and IL-22 and require the transcription factor RORγt^{2,17}. Th17 cells are involved in host defense against bacteria and fungi². Furthermore, CD4⁺ T cells can be induced in the periphery into CD4⁺ CD25⁺ Foxp3⁺ regulatory T cells and can produce TGF-β, IL-10 and IL-35. iTregs play a major role in the maintenance of self-tolerance and the prevention of autoimmunity and iTreg induction requires TGF-β, STAT5 and the transcription factor Foxp3^{8,18}.

To differentiate into Th1, Th2, Th17 or iTregs, naïve CD4^{|} T cells require a specific cytokine milieu³. Th1 induction requires the presence of IL-12 while IL-4 induces Th2 differentiation¹². It is well established that IFNγ produced by Th1 cells inhibits Th2 development while IL-4 produced by Th2 cells inhibits Th1 differentiation¹⁹. In addition to TGF-β, which is required for iTreg induction⁸, Th17 cells require IL-6, IL-21 and IL-23 for their differentiation and proliferation, respectively²⁰⁻²⁴. In addition to IFNγ and IL-4, which have been shown to inhibit Th17 differentiation, IL-2 has been shown to block Th17 cell development as well^{2,25-26}. Thus, for the past quarter century it is considered that cytokine milieu is critical and indispensable for naïve CD4⁺ T cell differentiation^{3,19,27}.

Herein, it is demonstrated that nicotinamide adenine dinucleotide (NAD⁺), a cofactor naturally found in the body and secreted during inflammation or under physiological conditions by different cell types such as epithelial cells, fibroblasts and neurons²⁸⁻³⁵, is able to regulate CD4⁺ T cell differentiation. NAD⁺ was able to regulate naïve CD4⁺ T cell differentiation in the absence of exogenous cytokines and more importantly had the capacity to override the effects of Th1, Th2 and iTreg polarizing conditions on T cell differentiation and cytokine production. Microarray analysis indicated that NAD⁺ had the capacity to activate tryptophan hydroxylase-1 (Tph-1) and carboxypeptidase A3 (CA3) during CD4⁺ T helper cell differentiation. Although intraperitoneal injection of NAD⁺ reduced Treg frequency and increased Th17 response, mice were still protected against experimental autoimmune encephalomyelitis (EAE), the most commonly used human model for multiple sclerosis, via Tr1 cells. The robust therapeutic potential of NAD⁺ was observed when NAD⁺ was administered after the disease onset and had the capacity not only to block but to reverse EAE progression by promoting myelin regeneration.

NAD⁺ Promotes a Robust IL-10 Response in Th1 Conditions but does not Affect IFN-γ Production

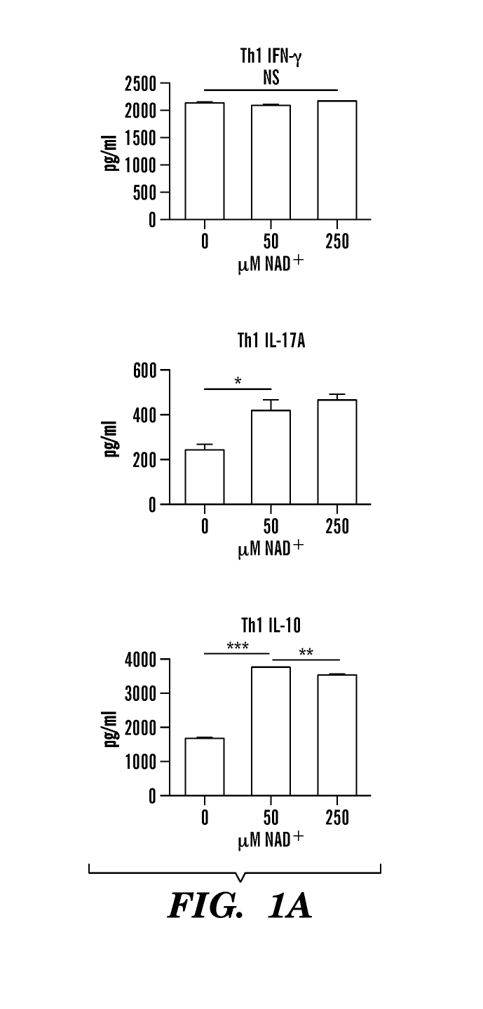

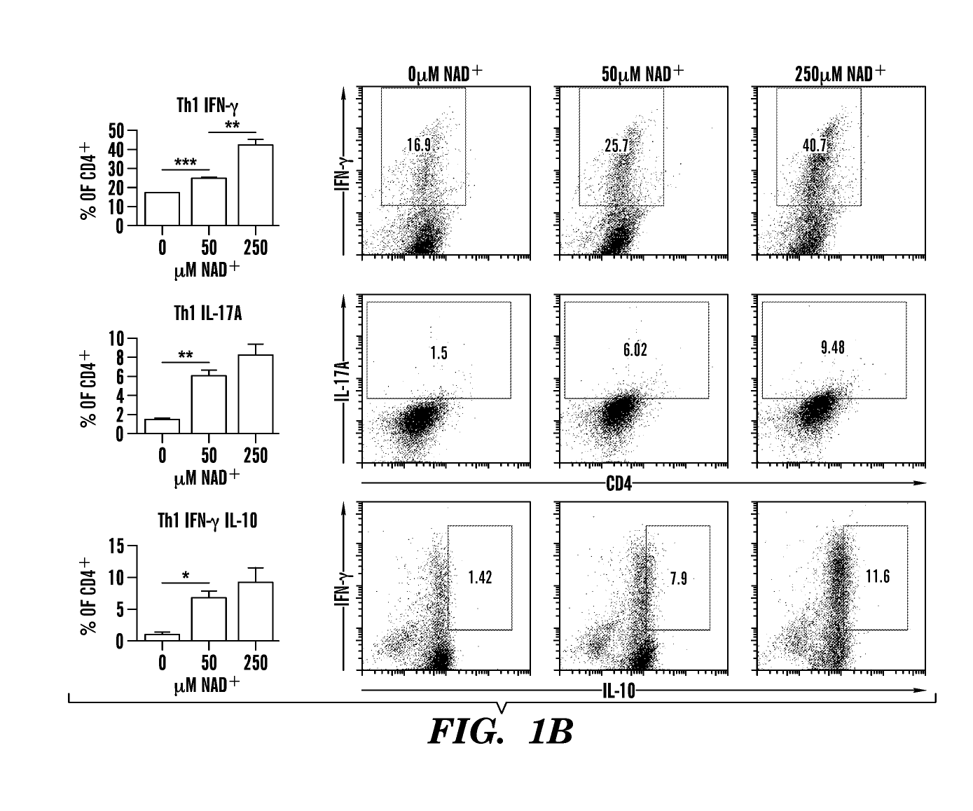

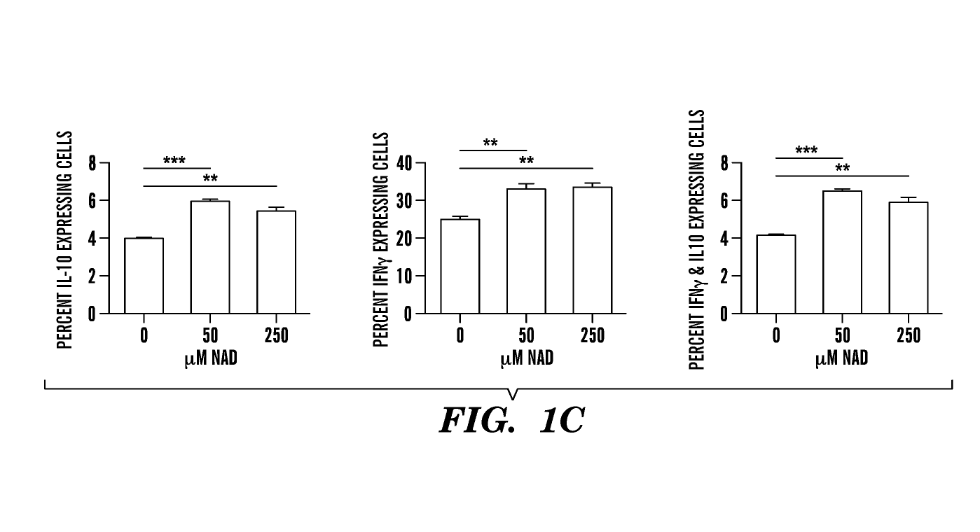

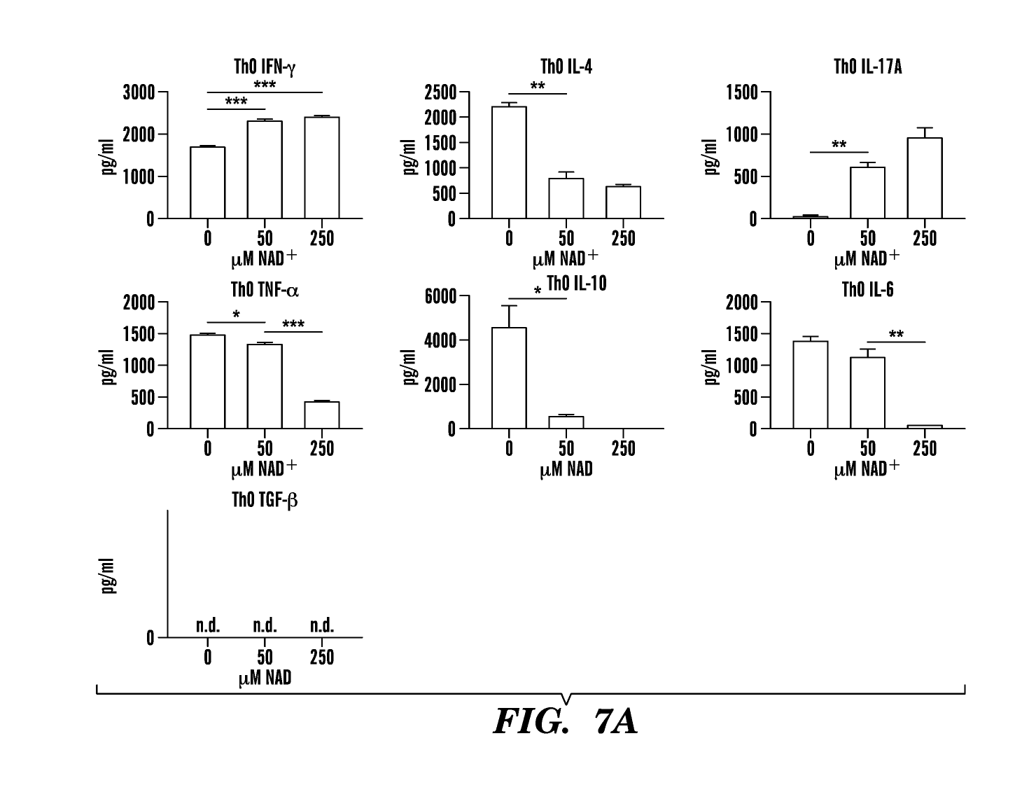

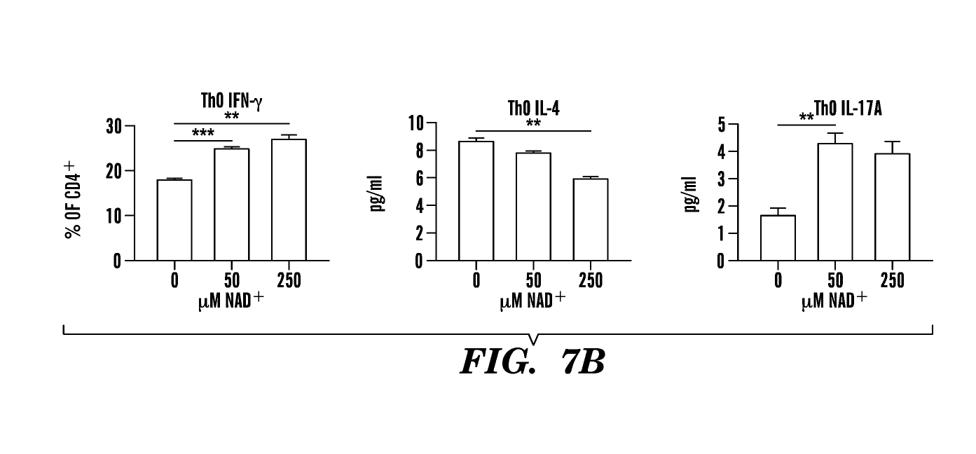

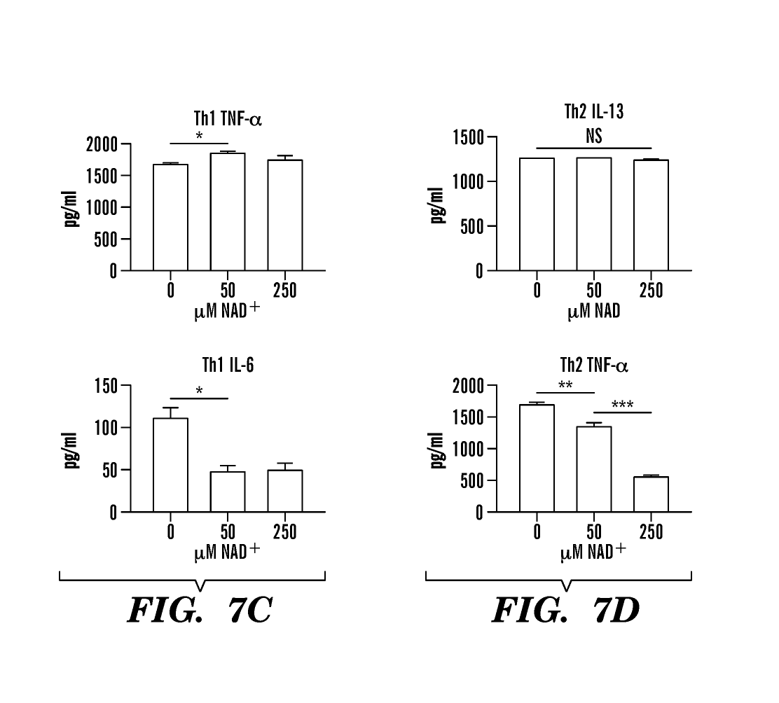

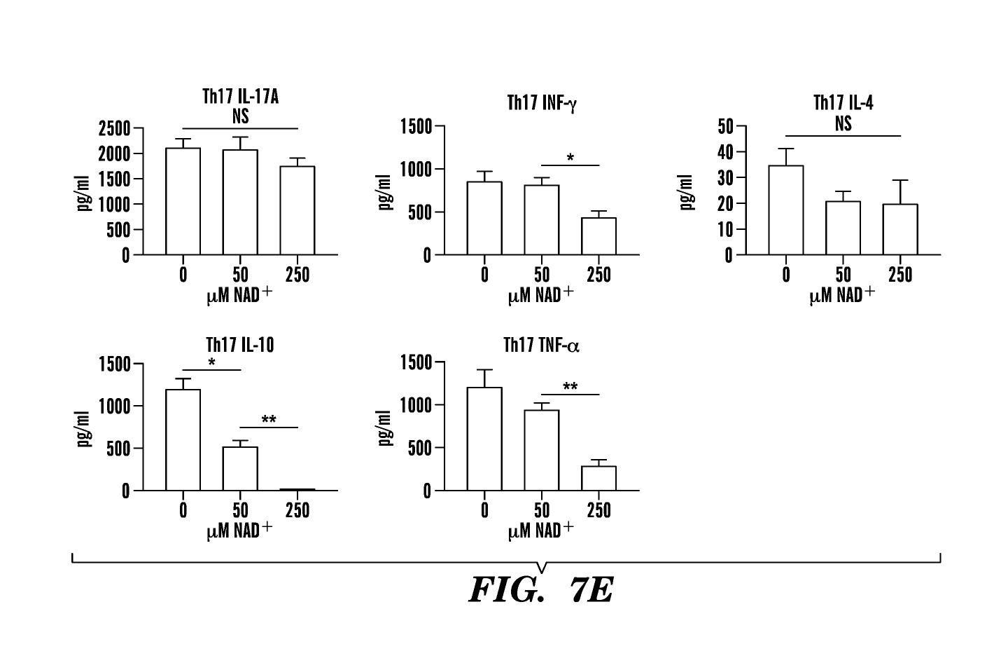

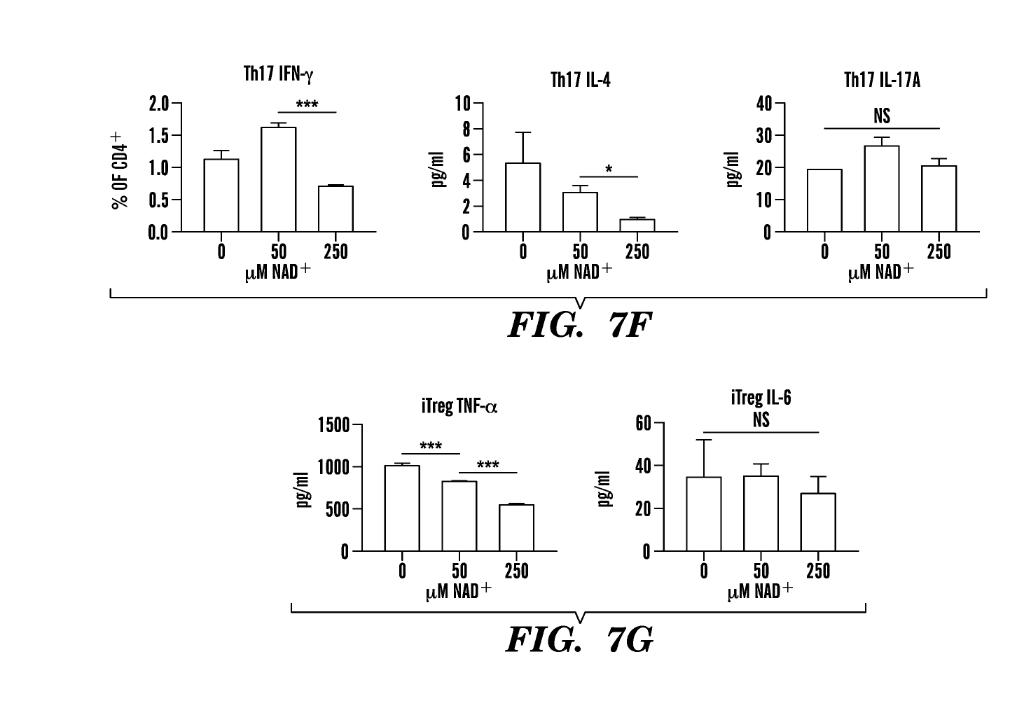

The inventors next investigated whether NAD⁻ regulates CD4⁺ T cell differentiation in Th1 polarizing conditions. Isolated naïve CD4⁺ T cells were cultured with anti-CD3/CD28 antibodies in presence of recombinant IL-12 and IL-2 cytokines and anti-IL4 antibody. In Th1 cytokine environment, NAD⁺ induced a moderate increase of IL-17A (400 pg/ml) but did not change IFN-γ cytokine production (FIG. 1A). In contrast to Th0 polarizing conditions, NAD⁺ induced a robust IL-10 secretion (>3500 pg/ml) in Th1 cytokine environment (FIG. 1A). Furthermore, pro-inflammatory cytokines such as TNF-α and IL-6 were reduced by NAD⁺ (FIG. 7C). These results were consistent with flow cytometry analyses. FIG. 1B showed increased frequencies of CD4⁺IFN-γ⁻, CD4⁺IL17A⁺ and CD4⁺IL-10⁺ cells. More importantly, flow cytometry analysis indicated that NAD⁺ promoted IL-10 production by mouse Th1-IFN-γ producing cells (FIG. 1B). Furthermore, the study tested whether NAD⁺ promoted the shift of human CD4⁺ T helper cells in Th1 polarizing conditions towards IL-10 cytokine production. Human peripheral blood CD4^{|} T cells were activated with anti-CD3/CD28 antibodies under Th1 polarizing conditions (recombinant IL-12 and IL-2 cytokines and anti-IL4 antibody) with increasing concentrations of NAD+. After 96 hours of culture, CD4⁺IL-10⁺IFNγ⁺ cell frequency was assessed by flow cytometry analysis. The results shown in FIG. 1C indicate that under Th1 polarizing conditions, NAD+ promotes a moderate increase in IL-17A that is accompanied by a robust IL-10 cytokine production by CD4⁺IFN-γ producing cells in mouse and human.

NAD⁺ Promotes Naïve CD4⁺ T Cell Differentiation into Th1 and Th17 Cells and Inhibits Th2 Differentiation

It has been shown that NAD⁺ can regulate T cell activation³⁶⁻³⁸, but its role in T cell development was not shown. In this study, it was tested whether NAD⁺ was able to regulate T cell differentiation in vitro. Naïve CD4⁺ T cells were isolated from spleen of DBA mice and cultured for 96 hrs in presence of increasing NAD⁺ concentration and in presence of recombinant IL-2 and stimulated with anti-CD3/CD28 antibodies. Cytokine expression and secretion were analyzed by flow cytometry and ELISA, respectively. FIG. 7A showed that NAD⁺ was able to promote a robust IFNγ, IL-17 cytokine production and to inhibit IL-4. Of note, the production of pro-inflammatory cytokines such as IL-6 and TNF-α or the immunosuppressive cytokine IL-10 were inhibited by NAD^{|} (FIG. 7A). Consistent with the ELISA results, flow cytometry analyses showed that NAD⁺ increased CD4⁺IFNγ⁺, CD4⁺IL-17⁺ and reduced CD4⁺IL-4⁺ cell frequencies (FIG. 7B). Taken together, these results indicate that NAD⁺ promotes naive CD4⁺ T helper cell differentiation into Th1 and Th17 cells and inhibits their conversion into Th2 cells.

NAD⁺ Drives the Switch of IL-4+ IL-10+ Th2 Cells into IL-17A+ Producing Cells and does not Affect Th17 Cell Differentiation

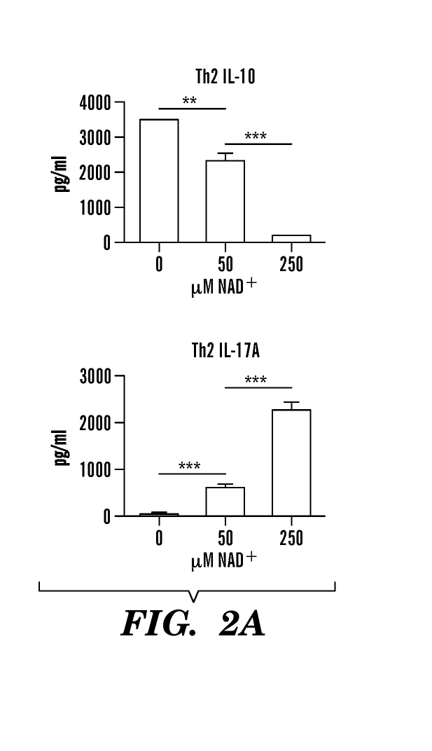

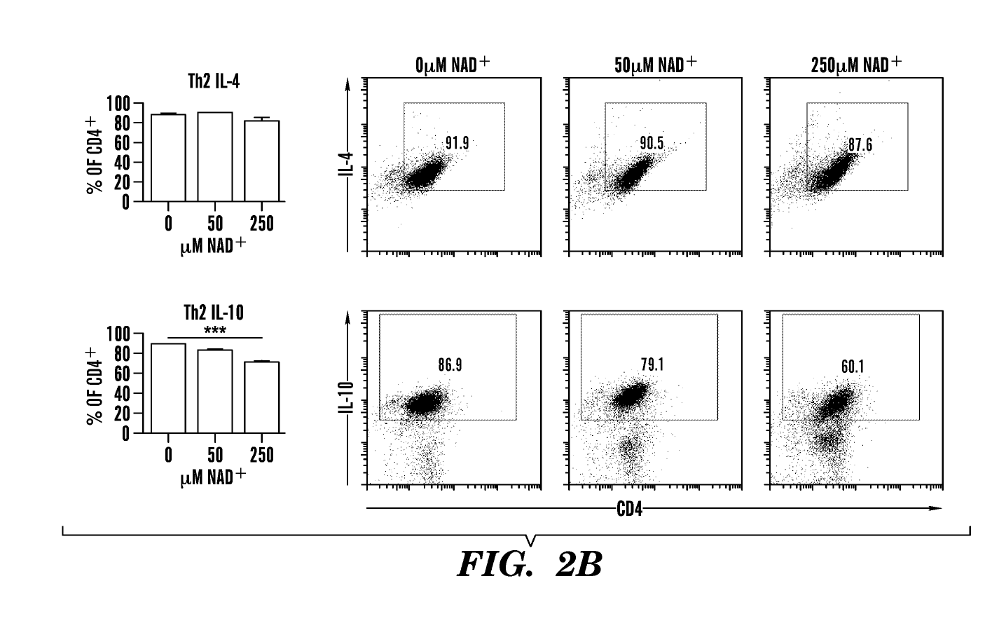

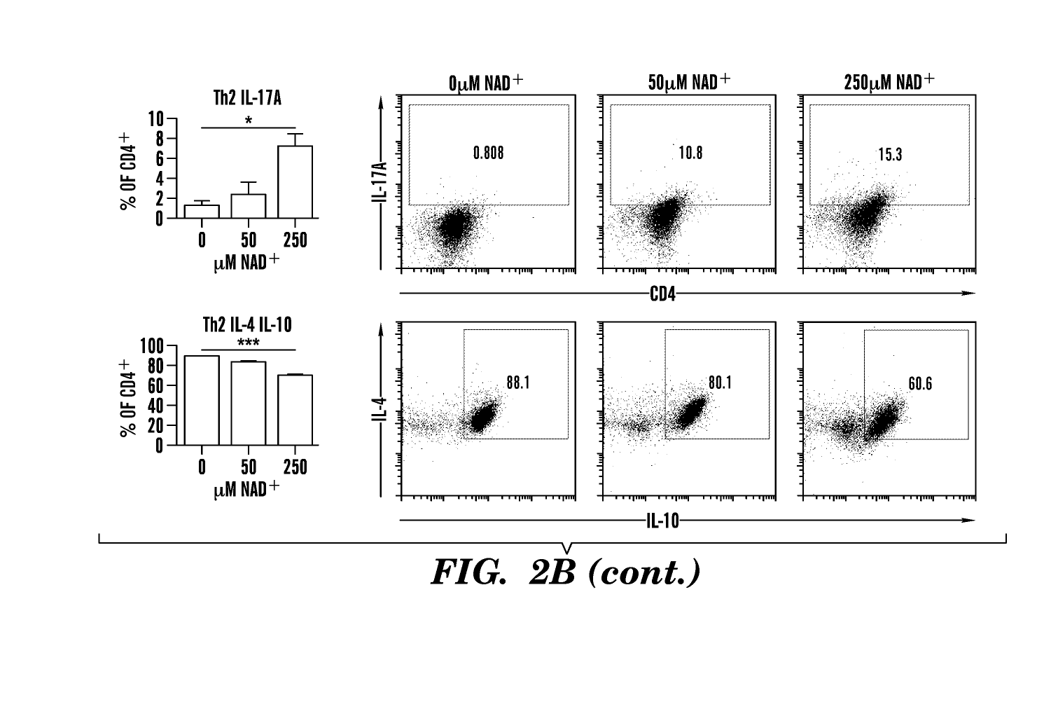

Next, the effects of NAD⁺ on activated CD4+ T cells were tested in Th2 polarizing conditions. In the Th2-promoting cytokine milieu, NAD⁺ did not affect IL-4 but reduced IL-10 cytokine production and induced a robust IL-17A secretion (FIG. 2A). In the absence of NAD⁻, CD4⁺ T cells cultured in Th2 cytokine environment barely produced IL-17A cytokine while in the presence of increasing concentrations of NAD⁺, cultured cells produced large amounts (>2000 pg/ml) in a dose dependent manner (FIG. 2A). Of note, increasing NAD⁻ concentrations did not affect IL-13 cytokine production, a cytokine initially considered produced mainly by Th2 cells (FIG. 7D). In contrast, production of the pro-inflammatory cytokine TNF-α was inhibited in the presence of NAD⁻ (FIG. 7D). Flow cytometry showed similar findings with no changes of CD4⁺IL-4⁺ and a decreased number of CD4⁺IL10⁺ cell frequency (FIG. 2B). Consistent with the ELISA findings an increased number of CD4⁺IL17A⁺ cells was observed (FIG. 2B). More importantly, flow cytometry results indicated that CD4⁺IL-4⁺ cells co-expressed IL-10 (FIG. 2B). Thus, these findings indicate that NAD⁺ drives the switch from classical Th2/IL-10⁺ toward Th2/IL-17A⁺ producing cells.

To elucidate the role of NAD⁺ on CD4⁻ T cell differentiations in Th17 polarizing conditions, isolated naïve CD4⁺ T cells were activated with anti-CD3/CD28 antibodies in the presence of recombinant TGFβ, IL-6 and anti-IL-4, anti-IL-12, and anti-IFNγ antibodies. The results indicated that NAD⁺ did not affect IL-17 (FIG. 1E) but reduced IL-10 cytokine production (FIG. 1E). Of note, expression of the pro-inflammatory cytokine TNF-α was inhibited in presence of NAD⁺ (FIG. 1E). Flow cytometry showed similar results with no changes of CD4⁺IL-17A⁺ cells (FIG. 1F). Collectively, these results indicate that NAD⁺ does not affect Th17 cell development.

NAD⁺ Promotes IL-17A and Reduces IL-10 Production under iTreg

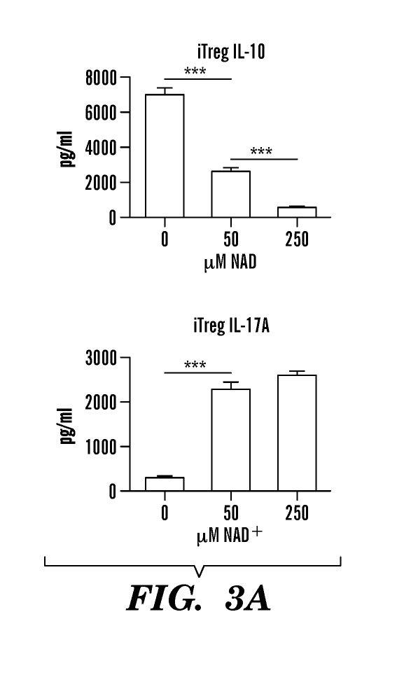

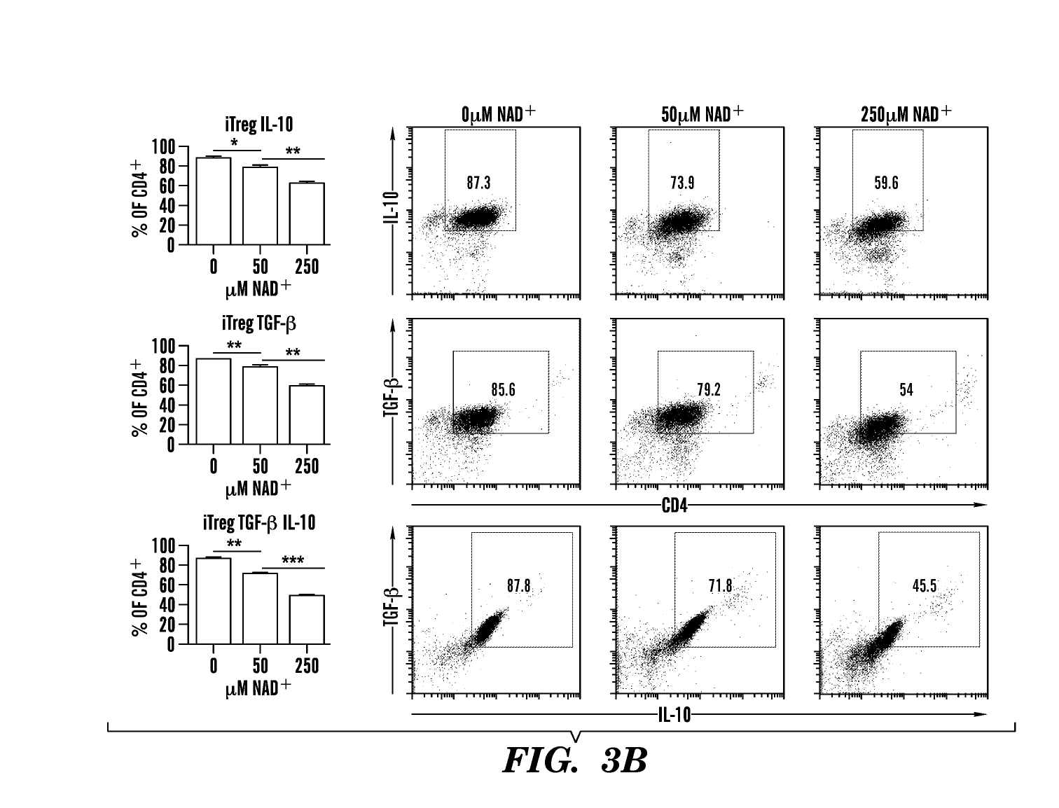

Next, it was investigated whether NAD⁺ modulates CD4⁺ T differentiation under iTreg polarizing conditions. Isolated naïve CD4⁺ T cells were activated with anti-CD3/CD28 antibodies in presence of recombinant TGFβ and IL-2 cytokines and blocking anti-IL-4, anti-IL-12 and anti-IFNγ antibodies. The results showed that NAD⁺ reduced IL-10 secretion, a hallmark cytokine of Tregs, and increased IL-17A cytokine production (FIG. 3A). Interestingly, in the presence of NAD⁺, production of the pro-inflammatory cytokine TNF-α was inhibited while IL-6 remained unchanged (FIG. 1G). Accordingly, flow cytometry analysis showed a robust decreased frequency of CD4⁺IL10⁺ and in parallel an increased frequency of CD4⁺IL17A⁺ cells (FIG. 3B). Moreover, a decreased number of CD4^{|}TGFβ^{|} IL-10⁺ cells were observed (FIG. 3B). Collectively, these results indicate that NAD^{|} promotes a robust conversion of iTregs into Th17 cells that overrides the previous inhibitory properties of IL-2 cytokine.

### NAD⁺ Protects Against EAE by Favoring Regulatory Type 1 Cell Differentiation and Myelin and Axonal Regeneration

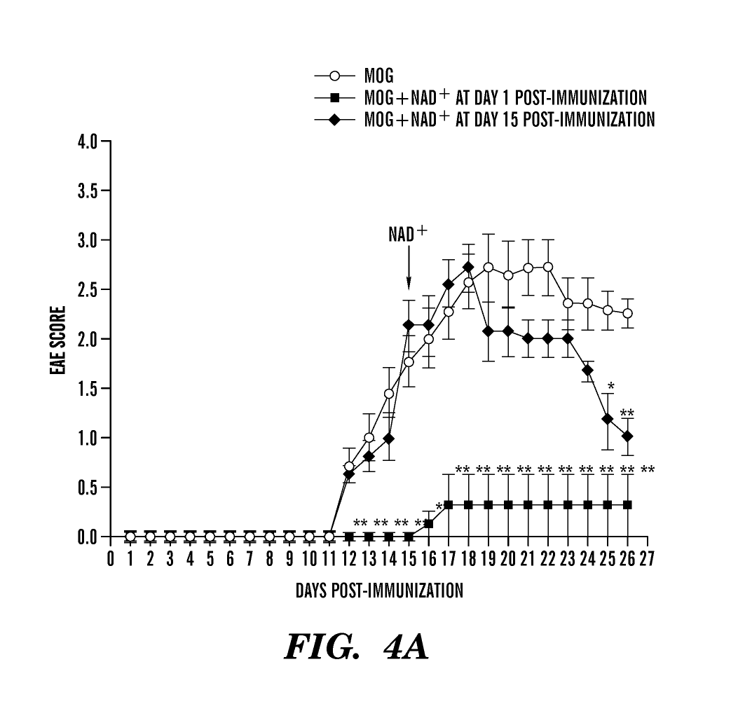

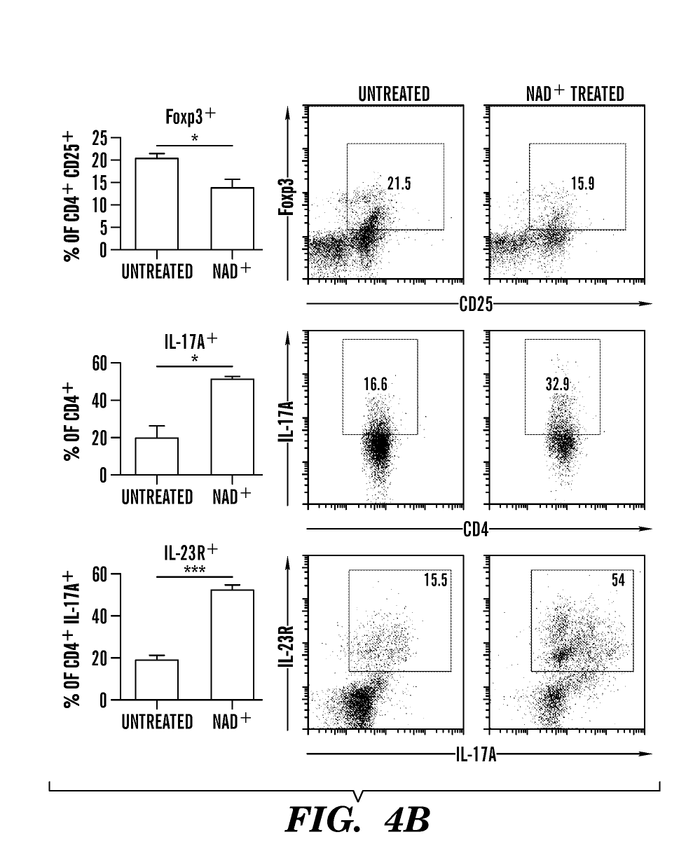

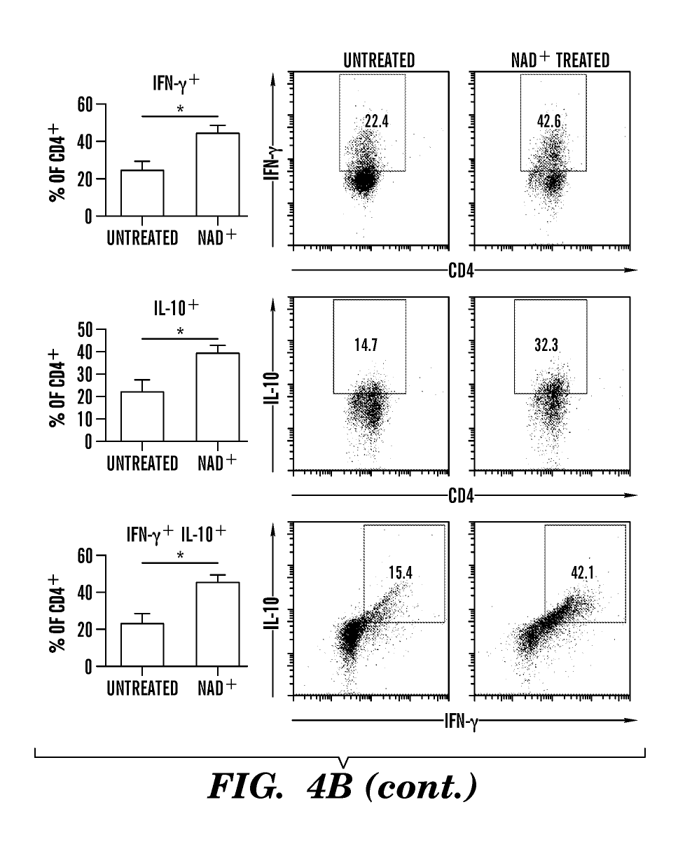

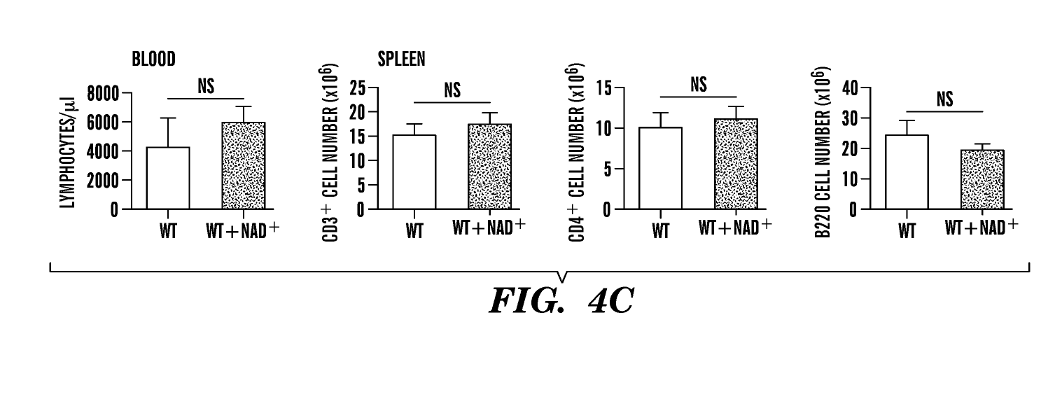

In vitro findings indicated that NAD⁺ favored Th1 development of naive CD4⁺ T cells, induces IL-10 cytokine production by Th1 cells and promotes Th17 response in Th2 and iTreg cytokine environment. Thus, the inventors used the experimental autoimmune encephalomyelitis (EAE) mouse model to study the impact of NAD⁺, in vivo, on IL-10, Th1, Th17 and Treg cells in vivo. EAE is a widely utilized model that recapitulates multiple sclerosis in humans and is known to be mediated by Th1 and Th17 cells.³⁹ Conversely, Tregs and IL-10 cytokine have been shown to play a major role in protection against and recovery from EAE⁴⁰⁻⁴². Onset of clinical signs of EAE in mice injected with MOG peptide occurred after 11 days and severe EAE was exhibited after 18 days (FIG. 4A) with a bilateral hindlimb paralysis (data not shown). In contrast, mice that received MOG peptide and were treated daily with NAD⁺ were protected against EAE (FIG. 4A) and exhibited no sign of paralysis. Moreover, NAD⁺ treated mice did not develop EAE even after 25 days (FIG. 4A) while non-treated animals exhibited severe symptoms (FIG. 4A). Next, the mechanisms by which NAD⁺ protected from EAE in vivo were investigated, in particular its impact on IL-10 cytokine, Th1, Th17 and Tregs. Spleens were isolated from mice treated with NAD⁺ or with a placebo solution 15 days after MOG immunization and CD4⁺ T cell cytokine expression profile was analyzed by flow cytometry. Consistent with a previous report³⁷, NAD⁺ treatment reduced the number of CD4⁺CD25⁺Foxp3⁺ cells (FIG. 4B). However, it was found that treatment with NAD⁺ promoted a robust Th17 response and IFNγ production by CD4⁺ T cells, which was consistent with the in vitro findings described herein (FIG. 4B). More importantly, NAD⁺ administration dramatically enhanced a systemic IL-10 cytokine production by CD4⁺ IFNγ⁺ cells when compared to the control group (FIG. 4B).

To assess the clinical efficacy of NAD⁺ as a potential robust therapeutic, a group of mice that developed severe clinical symptoms of EAE (e.g., bilateral hindlimb paresis) was subjected to a daily NAD⁺ treatment 15 days after MOG immunization. As shown in FIG. 4A, daily treatment with NAD⁺ 15 days after MOG immunization reversed the progression of the disease when compared to the control group of mice that was treated with a placebo solution (FIG. 4A). Treatment with NAD^{|} 15 days after MOG immunization rapidly abolished (within 10 days of treatment) the bilateral hindlimb while a majority of the non-treated mice continued to exhibit bilateral hindlimb paresis or weakness (FIG. 4A). Of note, NAD^{|} treatment in wild type mice did not affect the absolute number of circulating lymphocytes in the blood and spleens (FIG. 4C). Taken together these results indicate that NAD⁺ treatment is not only able to block EAE progression independently of Tregs or Th17 cells, through a robust systemic production of the immunosuppressive IL-10 cytokine by Th1 IFNγ-producing cells but to reverse rapidly EAE progression as well.

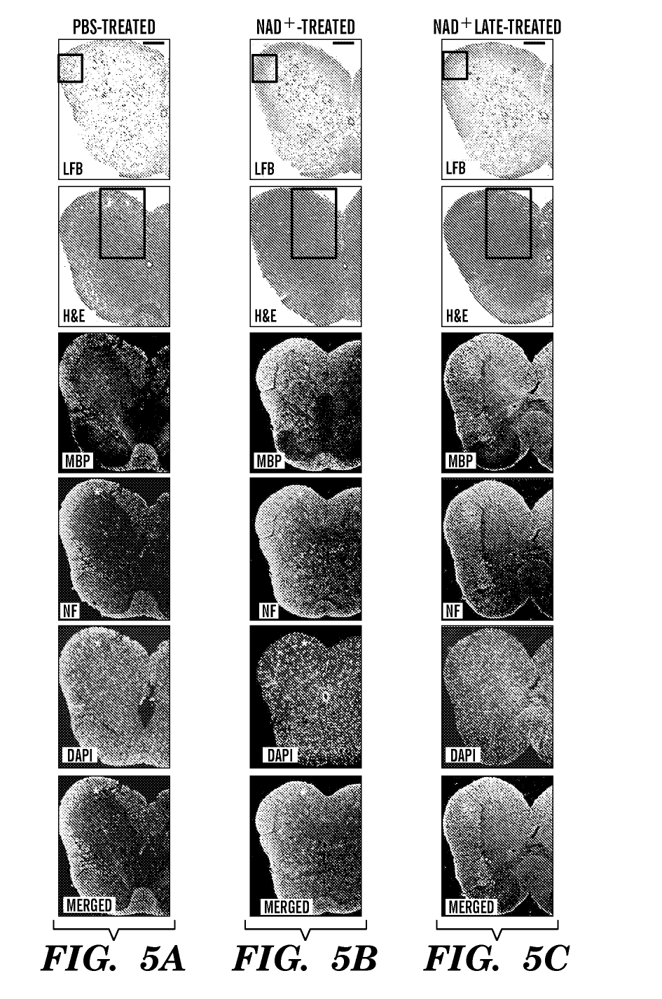

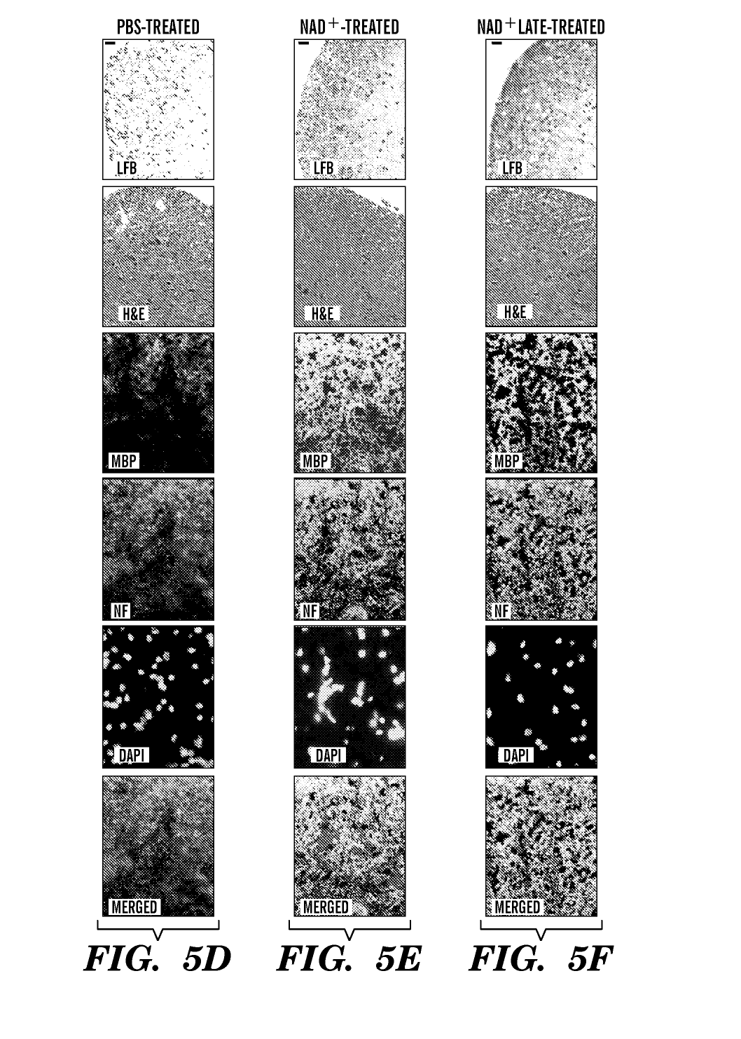

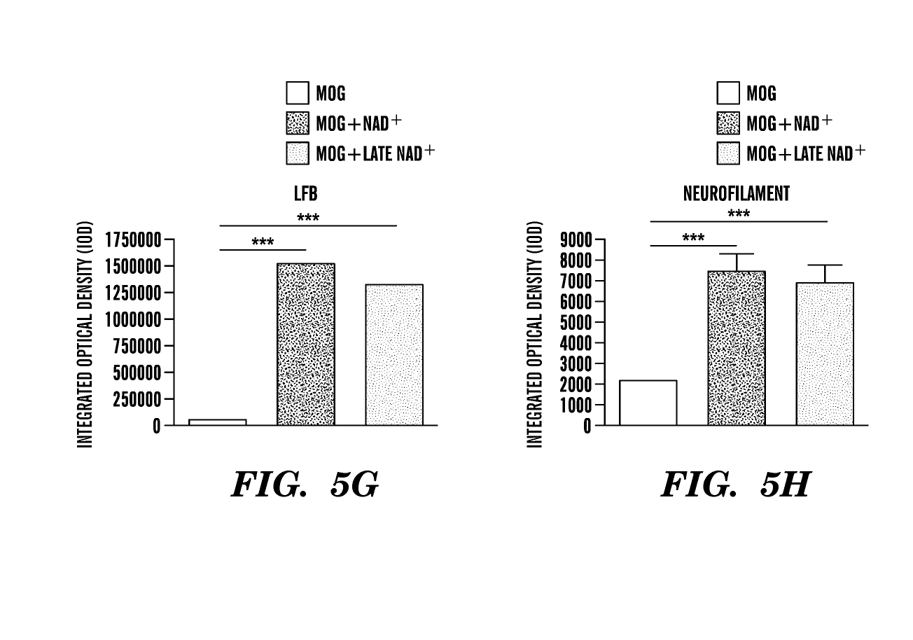

Thus, the mechanism of how NAD⁻ treatment was able to reverse EAE progression was investigated. A previous study reported that nicotinamide (Nam) an NAD biosynthesis precursor can protect from myelin degradation and axon degeneration⁴³. The results described herein indicate that NAD⁺ administered after the disease onset was able to reverse the progression of EAE. Thus, the inventors assessed whether NAD⁺ was able to reverse the clinical symptoms of EAE by protecting axon from degeneration and whether NAD⁺ had the capacity to promote myelin regeneration. The CNS of mice treated with a placebo solution revealed extensive inflammatory infiltrates of mononuclear cells and severe edema in the spinal cord 15 days after MOG immunization (FIG. 5A and 5C). Luxol fast blue staining showed dramatic and marked myelin loss and axonal injury (FIG. 5A). H&E staining revealed extensive inflammatory infiltrates of mononuclear cells in the spinal cords of mice treated with a placebo solution (FIGS. 5A and 5D). In contrast, the spinal cord of daily treated mice with NAD⁺ remained free of inflammatory infiltrate, myelin loss, and axonal injury in LFB and H&E stainings (FIG. 5A and 5C). More importantly, spinal cords of mice treated with NAD⁻ 15 days after MOG immunization (treated after hindlimb paralysis) did not show inflammatory infiltrate, myelin loss, and axonal injury in LFB and H&E stainings (FIG. 5A and 5C). Moreover, demyelination and axon loss were assessed with antibodies to myelin basic protein (MBP/SMI-9) and neurofilament (NF200), respectively, in all three groups (FIG. 5A-5F). Consistent with H&E and luxol blue findings, a significant reduction in both MBP staining depicting demyelination and axon loss evidenced by reduced NF 200 staining was very prominent in the group of mice treated with a placebo solution (FIG. 5A and 5D) when compared to the daily NAD^{|} treated group (FIG. 5B and 5E) and with mice treated with NAD+ after develop hindlimb paralysis (15 days after MOG immunization) (FIG. 5C and 5F). Collectively, these results indicate that NAD⁺ blocks EAE progression by protecting against myelin and axonal damage and more importantly that NAD⁺ reverses EAE progression by promoting myelin and axon regeneration.

NAD⁺ Regulates CD4+ T Helper Cell Differentiation and Protects from EAE Via Tph-1

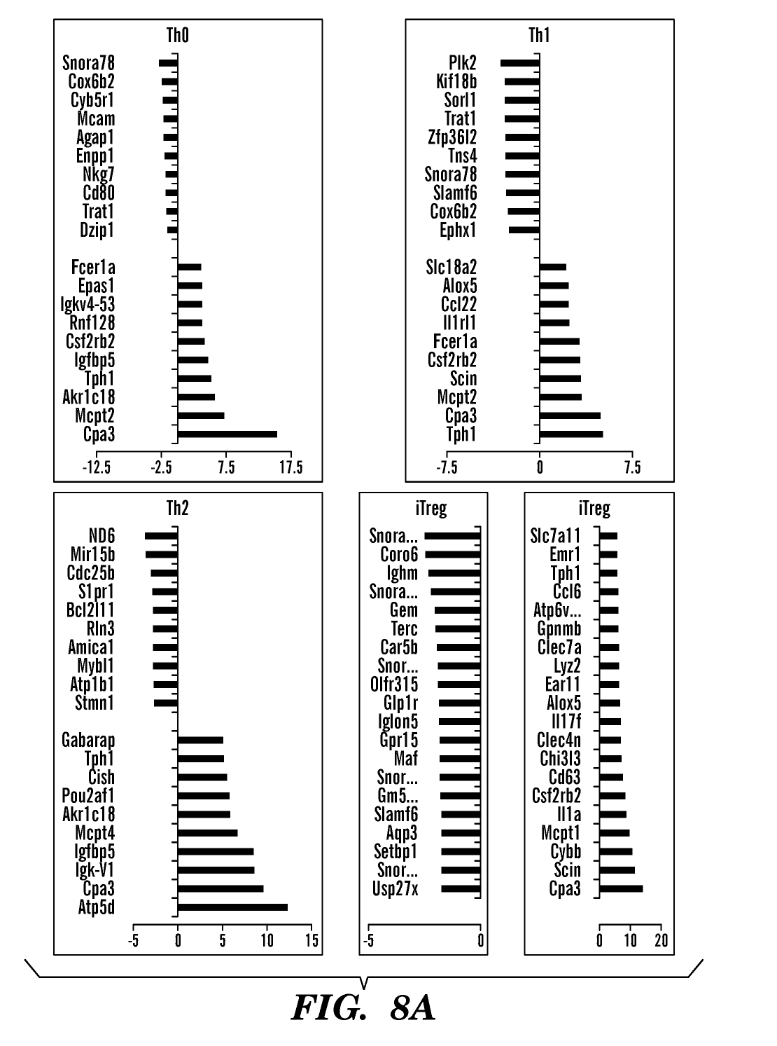

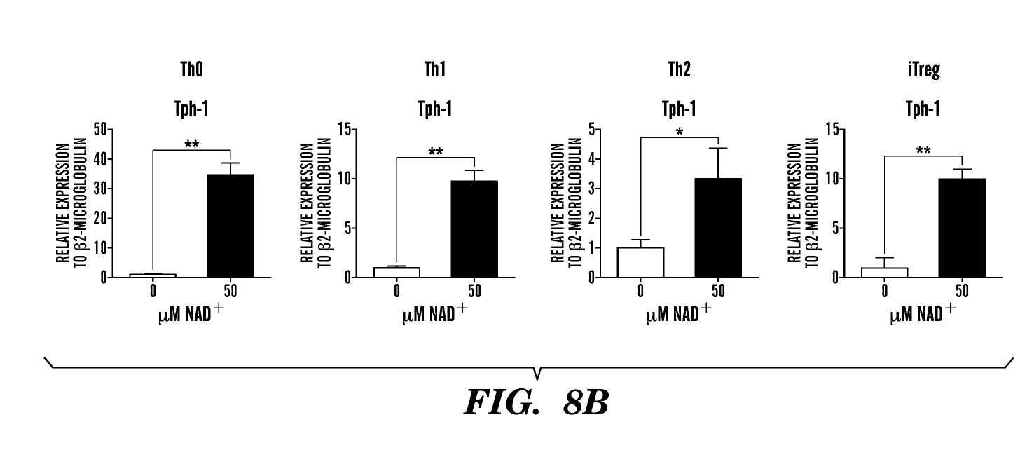

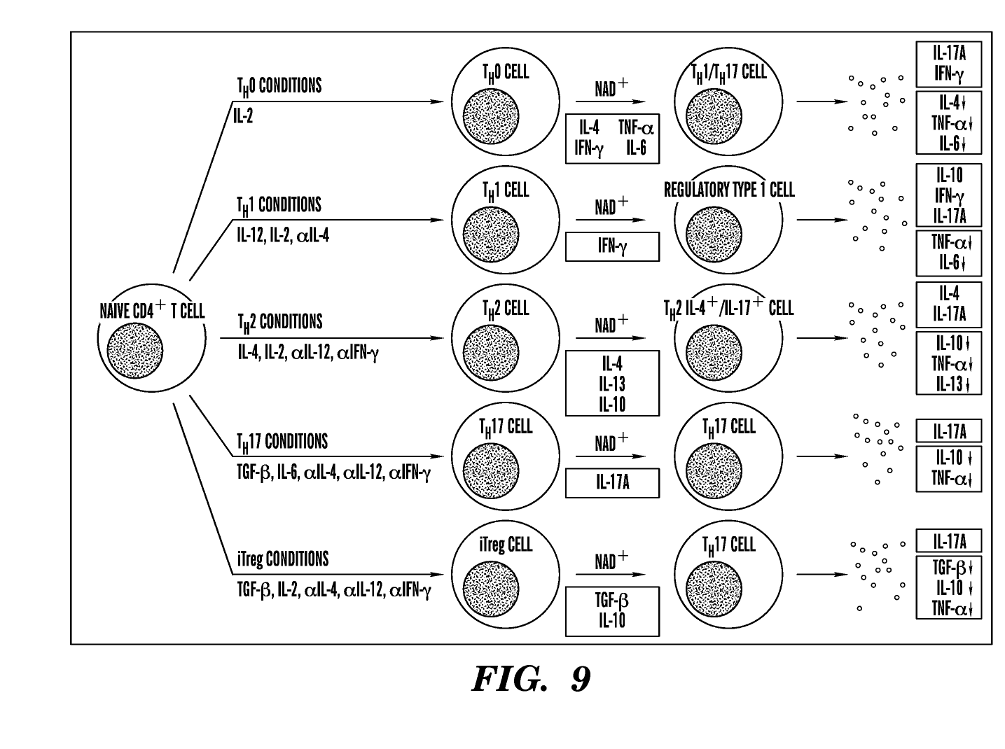

The inventors' findings indicated that NAD⁻ was able to regulate naïve CD4⁻ T cell differentiation in vitro and in vivo. More importantly, in vitro results indicated that NAD⁺ was able to override Th1, Th2 and iTreg but not Th17 polarizing conditions. Therefore, the gene expression profile was compared after treatment with or without NAD⁺ in Th1, Th2 and iTreg polarizing conditions. Thus, cells cultured under Th1, Th2 and iTreg polarizing conditions and in the presence or absence of NAD⁻ were collected after 96 hrs, RNA was extracted and transcriptional profiles were assessed by microarray analysis. Among the 20 genes up-regulated, Tph-1, a gene described initially as a mast cell gene⁴⁴ was found to be increased, in Th0, Th1, Th2 and iTreg polarizing conditions by microarray analysis (FIG. 8A). Furthermore, up-regulation of Tph-1 was confirmed by qPCR analysis (FIG. 8B). In addition, other pathways involved in T cell differentiation, IL-17 and IL-10 signaling were found to be activated by NAD⁺ (Table 1). Taken together, these results indicate that NAD⁺ may regulate CD4⁺ T helper differentiation through the Tph-1 and/or CA3.

Tph-1 has been very recently shown to prevent from allograft rejection and EAE⁴⁵. Thus, it was tested, in vivo, whether NAD⁺ protects from EAE and regulates CD4⁺ T cell differentiation through Tph1. Mice were subjected to EAE and were treated daily with a placebo solution (PBS) or NAD⁺ in addition to p-Chlorophenylalanine, a specific inhibitor of Tph-1.

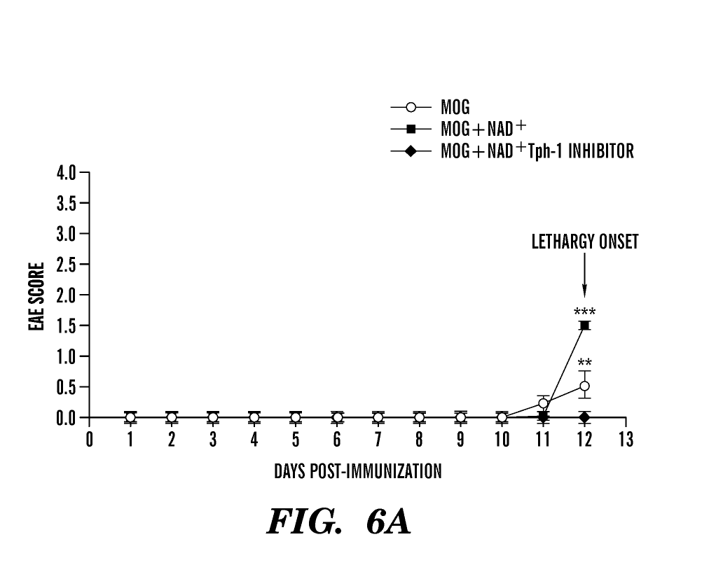

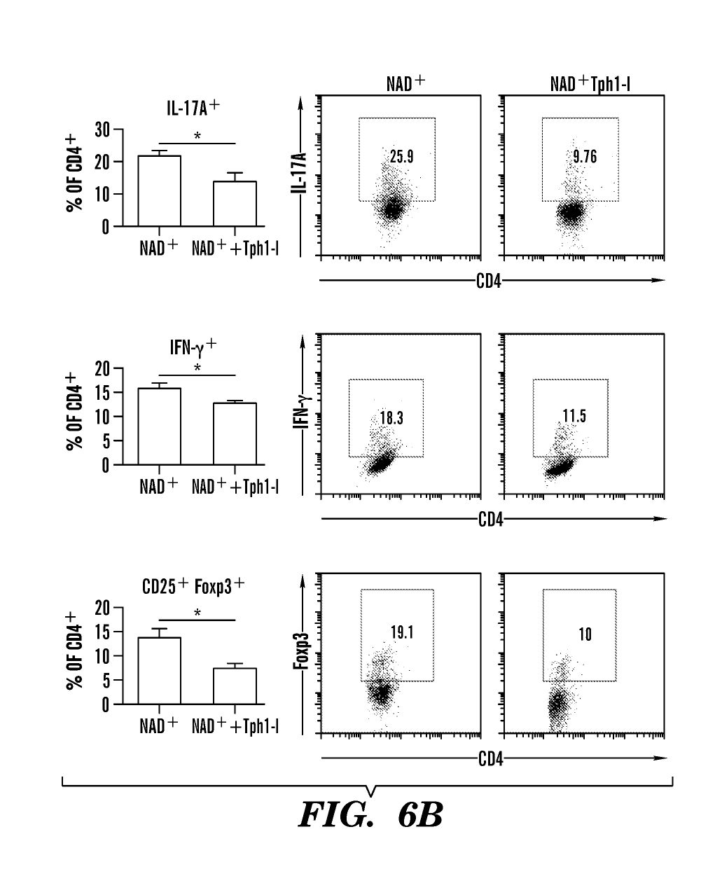

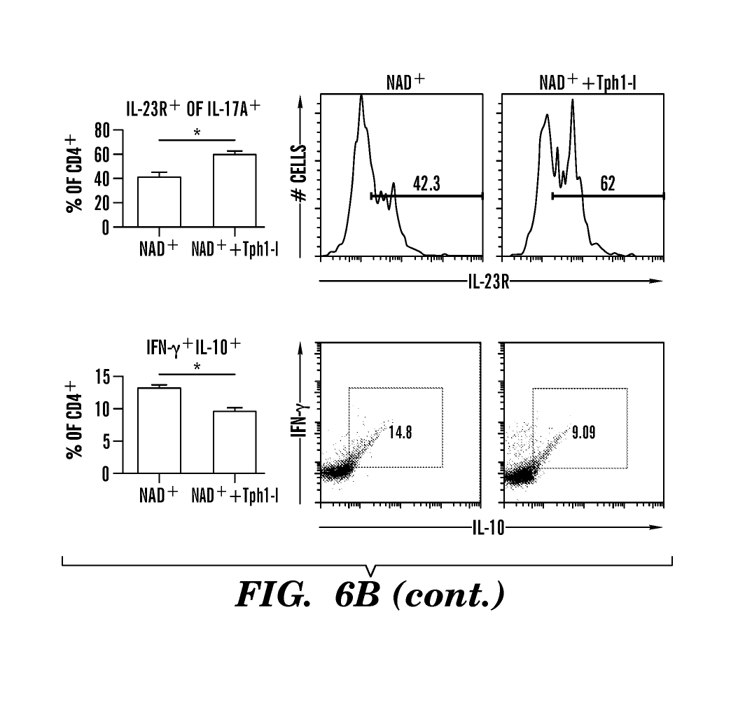

Onsets of clinical signs of EAE in mice injected with MOG peptide and a placebo solution appeared after 11 days (FIG. 6A). The inventors' previous results indicated that NAD⁺ was able to prevent from EAE (FIG. 4A). However, when NAD⁺ treated mice received simultaneously a treatment with a Tph-1 inhibitor, onset of clinical signs of EAE appeared after 11 days. Furthermore, 13 days after MOG immunization, mice treated with NAD+ and p-Chlorophenylalanine exhibited more severe clinical signs of EAE when compared to the group of mice that were subjected to MOG immunization and treated with a placebo solution (FIG. 6A). Mice treated with a Tph-1 inhibitor exhibited hindlimb paralysis and became rapidly lethargic. Flow cytometry indicated that NAD+ treatment followed by Tph-1 inhibition reduced systemically IL-17A and IL-10/IFNγ producing cells (FIG. 6B). Although Tph-1 inhibition reduced IL-17A+ cell frequency, the results indicated a significant increase of IL-17A+IL-23R+ cells (FIG. 6B). Moreover, Tph-1 inhibition resulted in a dramatic decrease of CD4+CD25+Foxp3+ cell frequencies. Taken together, these results indicate that Tph-1 play a critical role in CD4+ T helper cell activation and differentiation triggered by NAD+.

### Discussion

For the past quarter century, cytokine milieu has been considered the major determinant of T cell differentiation. As demonstrated herein, the inventors have uncovered a new, in vitro, differentiation pathway that promotes naïve CD4⁺ helper T cells towards Th1 and Th17 and inhibits Th2 development (data not shown). More importantly, these results are challenging the long-standing dogma of the “classical cytokine pathway”. It was shown that NAD⁺ had the capacity to override the effects of Th1, Th2 and iTreg polarizing conditions (data not shown). Under Th1 polarizing conditions and in presence of NAD⁺, human and mice CD4⁺ Th cells were able to rapidly secrete high amounts of IL-10 cytokine, originally considered a Th2 cytokine. It has been shown in previous studies that Th1 cells are able to produce IL-10; however it required several weeks (2-5 weeks) of high TCR stimulation⁴⁷ or a chronic Th1 activation¹³. NAD⁺ was able to induce IL-10 secretion by Th1 IFNγ-producing cells within hours without high TCR stimulation; indicating that NAD^{|} is a robust activator. In contrast, under Th2 polarizing conditions NAD^{|} reduced IL-10 cytokine production and promoted IL-17A cytokine production, indicating that NAD⁺ skews Th2 IL-10⁺ towards a Th2 IL-17A⁺ cells. Wang et al. have described a CD4⁺ Th2 subset that co-produces IL-4 and IL-17A cytokines and showed that these cells were the main cause of lung inflammation in the chronic stage of asthma. However, the mechanisms that promote IL-4⁺/IL-17A⁺ cell development remain unknown⁴⁸. The robust mechanism of action of NAD⁻ was confirmed, in vitro, with its capacity to convert iTregs into Th17 cells even in presence of IL-2, a cytokine that is known to inhibit Th17 commitment²⁵⁻²⁶. Although under Th0 polarizing conditions the inventors observed a decrease in IL-6 and no TGFβ production, two critical cytokines for Th17 differentiation, NAD+ was able Th17 differentiation (FIG. 1A and 1B and FIG. 7A). Furthermore, under iTreg polarizing conditions, NAD⁺ enhanced Th17 differentiation with a reduced amount of TGFβ (FIG. 5A and 5B) indicating that NAD^{|} does not promote T cell differentiation via cytokines. Therefore, these findings indicate that NAD+ is not only a robust CD4+ T helper regulator that can override the cytokine environment but can also promote a specific T cell differentiation depending on the T cell subset. Furthermore microarray analysis indicated an increase in Tph-1 expression by CD4+ T helper cells in all polarizing conditions, indicated that this enzyme may play an important role during CD4+ T cell differentiation after NAD+ activation. Indeed, in vivo treatment with Tph-1 dampened the systemic IL-17A and IL-10/IFNγ responses observed after NAD+ administration indicating that Tph-1 may play a critical role in T cell differentiation. In addition, mice treated with a Tph-1 inhibitor developed more severe clinical EAE signs than the group of mice that was subjected to MOG immunization and a placebo solution, indicating that, consistent with a previous study⁴⁵, Tph-1 plays a critical role in EAE protection. Mice treated with Tph-1 inhibitors develop severe lethargy 13 days after MOG immunization and had to be euthanized. The cause of death was most likely not the result of MOG immunization but of Tph-1 inhibition that has been shown to promote serotonin depletion, a crucial neurotransmitter⁴⁸, causing breathing difficulties, and inducing heart failure⁴⁹. The in vitro results described herein were consistent with the inventor's in vivo findings and showed that NAD+ was able to promote a robust systemic IL-10 cytokine production by Th1 cells and to promote a Th17 response.

Consistent with a previous study³⁷, NAD+ administration reduced the frequency of nTregs. Using an EAE disease mouse model, the animal model for human multiple sclerosis (MS), the inventors demonstrated that treatment with NAD+ not only protects from EAE but also had the capacity both to protect against EAE presumably through IL-10 despite the significant increase of IFNγ and TH17 response. IL-10 is a robust immunosuppressive cytokine that was shown to protect from EAE⁴². More importantly, when Th1 cells co-express IL-10, they have been shown to have immunosuppressive properties and to prevent exaggerated immune responses and concomitant tissue damage. Because of their anti-inflammatory properties, Th1 IL-10 producing cells were termed regulatory type 1 cells^{13-14,16}. However, the mechanisms that promote IL-10 cytokine production by Th1 cells remain poorly understood. In line with the inventors' findings, it was recently shown that the conversion from IFNγ to IL-10 production by Th1 cells can prevent tissue damage and autoimmune diseases¹⁴⁻¹⁵. The development of EAE clinical signs in mice treated with NAD+ and Tph-1 inhibitor may result from the reduced frequency of Th1 IL-10 producing cells and the increased frequency of CD4+IL-17A+IL-23R+ cells that have been shown to be more pathogenic⁵⁰.

Furthermore, NAD+ might favor this process by inhibiting the inflammatory response or might act on other pathways involved in the central nervous system. Thus, the study described herein unravels a new mechanism of CD4+ T helper cell differentiation and underscores the therapeutic potential of NAD+ in autoimmune diseases such as EAE, Type 1 diabetes, and inflammatory bowel diseases in which IL-10 has been shown to play a central role⁵¹⁻⁵².

### Materials and Methods

### Animals

Eight to ten week old C57BL/6 and DBA/2 mice were purchased from Charles River Laboratories (Wilmington, Mass.) Animal use and care was in accordance with institutional and National Institutes of Health guidelines.

Isolation of Naïve CD4⁺ T Cells and Cell Culture

Single-cell leukocyte suspensions were obtained from spleens from 8-10 week old DBA mice and CD4⁻ T cells were isolated by negative selection using a CD4⁺ T cell isolation kit (MILTENYI BIOTEC, Bergisch Gladbach, Germany). Cells were further sorted using α-CD4-PE (EBIOSCIENCE, San Diego, Calif.) and purities of CD4⁺ T cells after isolation were >98%.

To isolate human naïve CD4⁺ T cells, peripheral blood mononuclear cells were obtained from young healthy volunteers using density gradient centrifugation with Ficoll-Paque (STEMCELL TECHNOLOGIES, Vancouver, BC, Canada) and naïve CD4⁺ T cells were negatively isolated using anti-biotin magnetic beads (MILTENYI BIOTEC). Purities of T reg cells were >98%.

Isolated CD4⁺ T cells were cultured in 24-well flat bottom plates (0.5×10⁶ cells per well) in 0.5 ml of complete RPMI 1640 media (supplemented with 10% FCS, 200 mM L-glutamine, 100 U/ml penicillin/streptomycin and 5×10⁻⁵ M 2-mercaptoethanol (RP-10) in the presence of 10 μg/ml plate-bound anti-mouse α-CD3 (17A2) and 2 μg/ml soluble α-CD28 (37.51) in addition to 50 ng/ml recombinant mouse IL-2 (all EBIOSCIENCE). NAD⁺ (SIGMA-ALDRICH) was added as indicated. Cells were cultured in polarizing Th1 (20 ng/m1 of recombinant IL-12 and 10 μg/ml of anti-IL-4), Th2 (20 ng/ml of recombinant IL-4, 10 μg/ml of anti-IFNγ), Th17 (10 ng/ml of recombinant TGF-β, 100 ng/ml of recombinant IL-6, 10 μg/ml of anti-IFNγ, and 10 μg/ml of anti-IL4) or iTreg (10 ng/ml of recombinant TGFβ, 10 μg/ml of anti-IFNγ, and 10 μg/ml of anti-IL4) conditions. All recombinant cytokines and antibodies were purchased from EBIOSCIENCE except recombinant TGF-β cytokine (R&D SYSTEMS, Minneapolis, Minn.). After 96 hrs of culture supernatants and cells were collected and analyzed by ELISA and flow cytometry, respectively.

Flow cytometry

Fluorescently labeled anti-mouse α-CD4 (GK1.5), α-CD25 (PC61), α-IFN-γ (XMG1.2) and unlabeled α-CD16/CD32 (2.4G2) antibodies were obtained from BD BIOSCIENCES (San Jose, Calif.). Fluorescently labeled anti-mouse α-IL-4 (FJK-16s), α-IL-10 (JES5-16E3), α-IL-17A (eBio17B7), and anti-TGFβ (TW7-16B4) were obtained from eBIOSCIENCE (San Diego, Calif.). Intracellular staining for IL-4, IL-10, IL-17A, IFNγ and TGFβ was performed according to manufacturers' protocols. Splenocytes were re-stimulated in complete media (RPMI media containing 10% FCS, 1% L-Glutamine, 1% Penicillin/Streptomycin; all Bio Whittaker, Walkersville, Md.) for 4 hours at 37° C. with ionomycin, phorbol 12-myristate 13-acetate and Brefelding A (EBIOSCIENCE). Cells were fixed and permeabilized using CYTOFIX/CYTOPERM solution (BD BIOSCIENCES). Flow cytometry measurements of single-cell suspensions were performed on a FACSCalibur using standard procedures and data were analyzed using FLOWJO software (TREE STAR, Ashland, Oreg.).

### ELISA

Mouse IL-4, IL-6, IL-10, IL-13, IL-17A, IFNγ, TNFα and TGFβ were measured using commercial kits (EBIOSCIENCE). Briefly, ELISA plates were coated with 100 μl of anti-cytokine capture antibody at 4° C. overnight. Plates were then washed ×5 with 0.05% PBS-Tween (PBST) and coated for 1 h with the blocking buffer provided by the manufacturer. Samples or standards were added in duplicates (100 μl/well) and incubated at 4° C. overnight. Wells were washed ×5 with PBST and incubated with 100 μl of anti-cytokine detection antibody at 4° C. overnight. Wells were then washed ×5 with PBST and incubated with 100 μl of avidin-HRP at room temperature for 30 min. Thereafter, wells were washed ×7 with PBST and incubated with 100 μl/well of a substrate. The reaction was stopped after 15 min with 1M H₂SO₄ and absorbance was measured using a multiplate microplate fluorescence reader (Synergy HT, Biotek, Winooski, Vt.) at 405 nm.

### RNA Extraction and Microarray Analyses

RNA was extracted from CD4⁺ T cells cultured in Th0, Th1, Th2 and iTreg polarizing conditions after 96 hrs using the RNAqueous extraction kit according to the manufacturer's protocols (Applied Biosystems, Carlsbad, Calif.). Briefly, cells were homogenized in lysis buffer (total volume of 0.5 ml) and passed through a column. After successive washes, RNA was eluted. Microarray analyses were performed using an AFFYMETRIX genechip. Briefly, the cDNA (10 μg) was fragmented and labeled and then was hybridized to a GeneChip Mouse Genome 430 2.1 array (AFFYMETRIX). AFFYMETRIX microarray data analysis was performed using PARTEK GENOMICS SUITE software. Default settings were used for quantile normalization and RMA summarization. The samples were grouped and statistics were applied using the ANOVA model employing linear contrast. Linear contrast was established for each pair of grouped samples for which the analysis was appropriate. Gene lists were then created for each analysis for downstream pathway analysis using threshold values of FDR-adjusted p-value ≦0.05 and fold change ≧2 and ≦2. The Ingenuity Pathways Analysis (INGENUITY SYSTEMS®) applications were used to generate canonical pathways associated with the differentially expressed gene profiles extracted from the transcriptome data.

TABLE 1
Canonical Pathways

| | |
|---|---|
| Ingenuity Canonical Pathways | −log(p-value) |

Th0

| | |
|---|---|
| Eicosanoid Signaling | 2.56E00 |
| VDR/RXR Activation | 2.54E00 |
| Methylglyoxal Degradation III | 2.51E00 |
| Androgen Biosynthesis | 2.41E00 |
| Crosstalk between Dendritic Cells and Natural Killer Cells | 2.38E00 |
| Communication between Innate and Adaptive Immune Cells | 2.37E00 |
| CTLA4 Signaling in Cytotoxic T Lymphocytes | 2.36E00 |
| B Cell Development | 2.23E00 |
| Retinoate Biosynthesis I | 2.13E00 |
| iCOS-iCOSL Signaling in T Helper Cells | 2.12E00 |
| Serotonin Receptor Signaling | 1.98E00 |
| Graft-versus-Host Disease Signaling | 1.91E00 |
| Autoimmune Thyroid Disease Signaling | 1.87E00 |
| Bile Acid Biosynthesis, Neutral Pathway | 1.87E00 |
| Hepatic Fibrosis/Hepatic Stellate Cell Activation | 1.86E00 |

Th1

| | |
|---|---|
| Mitotic Roles of Polo-Like Kinase | 3.95E00 |
| Cell Cycle: G2/M DNA Damage Checkpoint Regulation | 3.26E00 |
| IL-10 Signaling | 2.68E00 |
| T Helper Cell Differentiation | 2.66E00 |
| IL-3 Signaling | 2.63E00 |
| FLT3 Signaling in Hematopoietic Progenitor Cells | 2.58E00 |
| Acute Myeloid Leukemia Signaling | 2.53E00 |
| Asparagine Biosynthesis I | 2.43E00 |
| IL-17A Signaling in Gastric Cells | 2.41E00 |
| NRF2-mediated Oxidative Stress Response | 2.34E00 |
| Serotonin Receptor Signaling | 2.17E00 |
| iCOS-iCOSL Signaling in T Helper Cells | 2.13E00 |
| IL-17A Signaling in Fibroblasts | 2.12E00 |
| Molecular Mechanisms of Cancer | 2.07E00 |
| April Mediated Signaling | 2.05E00 |

Th2

| | |
|---|---|
| Primary Immunodeficiency Signaling | 6.78E00 |
| B Cell Development | 3.94E00 |
| FcγRIIB Signaling in B Lymphocytes | 3.52E00 |
| Hematopoiesis from Pluripotent Stem Cells | 3.15E00 |
| Remodeling of Epithelial Adherens Junctions | 2.68E00 |
| Role of NFAT in Regulation of the Immune Response | 2.63E00 |
| Phospholipase C Signaling | 2.61E00 |
| p70S6K Signaling | 2.59E00 |
| Atherosclerosis Signaling | 2.58E00 |
| PI3K Signaling in B Lymphocytes | 2.45E00 |
| Altered T Cell and B Cell Signaling in Rheumatoid Arthritis | 2.32E00 |
| Antioxidant Action of Vitamin C | 2.16E00 |
| B Cell Receptor Signaling | 2.07E00 |
| Differential Regulation of Cytokine Production in | 2.06E00 |
| Macrophages and T Helper Cells by IL-17A and IL-17F | |
| Granzyme A Signaling | 2.01E00 |

iTreg

| | |
|---|---|
| Differential Regulation of Cytokine Production in | 1.13E01 |
| Macrophages and T Helper Cells by IL-17A and IL-17F | |
| Differential Regulation of Cytokine Production | 1.02E01 |
| in Intestinal Epithelial Cells by IL-17A and IL-17F | |
| Graft-versus-Host Disease Signaling | 8.88E00 |
| Altered T Cell and B Cell Signaling in Rheumatoid Arthritis | 7.61E00 |
| Communication between Innate and Adaptive Immune Cells | 7.37E00 |
| T Helper Cell Differentiation | 7.29E00 |
| LXR/RXR Activation | 7.01E00 |
| Role of Hypercytokinemia/hyperchemokinemia in the Pathogenesis of Influenza | 6.44E00 |
| Dendritic Cell Maturation | 6.42E00 |
| Role of Cytokines in Mediating Communication between Immune Cells | 5.76E00 |
| Atherosclerosis Signaling | 5.25E00 |
| IL-10 Signaling | 4.96E00 |
| Role of NFAT in Regulation of the Immune Response | 4.74E00 |
| Role of Osteoblasts, Osteoclasts and Chondrocytes in Rheumatoid Arthritis | 3.88E00 |
| Type I Diabetes Mellitus Signaling | 3.8E00 |

### EAE Mouse Model

EAE was induced in 10-week-old C57BL/6 mice using Hooke Labs EAE induction kit (EK-2110) according to manufacturer's instructions. Mice were scored daily on a scale of 0 to 5 in a double-blind manner with the following criteria: score 0, no signs of neurological disease; score 1, flaccid paralysis of the tail; partial or no tail muscle tone mouse is unable to curl tail around finger or pencil; score 2, hindlimb paresis weak or wobbling gait and/or impaired righting reflex; score 3 bilateral hindlimb paresis mouse drags its hindlimbs over flat surface and/or exhibits incontinence; score 4, hind and forelimb paralysis mouse barely moves around; score 5, moribund animal). For NAD⁺ treatment mice received a daily intraperitoneal injection (60 mg in 100 μl in PBS) or a placebo solution (100 μl of PBS) at the same day than MOG immunization. Mice were sacrificed at day 18 and single cell suspension was obtained from spleens for flow cytometry analysis. To test whether NAD⁺ was able to reverse the development of EAE, another group of mice was treated daily (for a period of 10 days) with NAD⁺ (60 mg/mouse) or a placebo solution 15 days after MOG immunization when mice were scored 3 with a bilateral hindlimb paresis. EAE disease progression was monitored. For Tph-1 or CA3 inhibition, mice received daily intraperitoneal injections (simultaneous to NAD⁻) of p-Chlorophenylalanine (1 ml of a 10 mM solution stock, catalog number 0938, Tocris Bioscience) or N-[[[(1S)-1-Carboxy-3-methylbutyl]amino]carbonyl]-L-glutamic acid (100 μl of a 100 mM solution stock, ZJ 43 catalog number 2675,Tocris Bioscience), respectively.

### Count of Absolute Number of Lymphocytes

C57BL/6 mice (8-10 weeks old) were daily administered 60 mg of NAD⁺ or a placebo solution (PBS) intraperitoneally during 4 days. Blood and spleen were collected after 4 days of treatment. Absolute number of lymphocytes in the blood was determined using a hemocytometer (model 850 FS, Drew Scientific, Miami lakes, Fla.). To determine absolute lymphocyte number in spleens, cells were isolated and counted with a hemocytometer then stained with antibodies anti-CD4⁺, CD3⁺ or B220⁺ and percentage was determined by flow analysis.

### Statistical Analysis

Values and error bars represent mean ± SEM. For comparison, the Mann-Whitney U test was used. All values were analyzed using GRAPHPAD PRISM (GRAPHPAD Software, San Diego, Calif.). For microarray analysis ANOVA was used. A p value <0.05 was considered statistically significant.

### REFERENCES

1. Sakaguchi, S. Control of immune responses by naturally arising CD4+ regulatory T cells that express toll-like receptors. J Exp Med 197, 397-401 (2003).
2. Martinez, G. J., Nurieva, R. I., Yang, X. O. & Dong, C. Regulation and function of proinflammatory TH17 cells. Ann N Y Acad Sci 1143, 188-211 (2008).
3. Zhu, J., Yamane, H. & Paul, W. E. Differentiation of effector CD4 T cell populations (*). Annu Rev Immunol 28, 445-489 (2010).
4. Mosmann, T. R., Cherwinski, H., Bond, M. W., Giedlin, M. A. & Coffman, R. L. Two types of murine helper T cell clone. I. Definition according to profiles of lymphokine activities and secreted proteins. J Immunol 136, 2348-2357 (1986).
5. Mosmann, T. R. & Coffman, R. L. Heterogeneity of cytokine secretion patterns and functions of helper T cells. Adv Immunol 46, 111-147 (1989).
6. Mosmann, T. R. & Coffman, R. L. TH1 and TH2 cells: different patterns of lymphokine secretion lead to different functional properties. Annu Rev Immunol 7, 145-173 (1989).
7. Killar, L., MacDonald, G., West, J., Woods, A. & Bottomly, K. Cloned, Ia-restricted T cells that do not produce interleukin 4(IL 4)/B cell stimulatory factor 1(BSF-1) fail to help antigen-specific B cells. J Immunol 138, 1674-1679 (1987).
8. Josefowicz, S. Z., Lu, L. F. & Rudensky, A. Y. Regulatory T cells: mechanisms of differentiation and function. Annu Rev Immunol 30, 531-564 (2012).
9. Park, H., et al. A distinct lineage of CD4 T cells regulates tissue inflammation by producing interleukin 17. Nat Immunol 6, 1133-1141 (2005).
10. Harrington, L. E., et al. Interleukin 17-producing CD4+ effector T cells develop via a lineage distinct from the T helper type 1 and 2 lineages. Nat Immunol 6, 1123-1132 (2005).
11. Abbas, A. K., Murphy, K. M. & Sher, A. Functional diversity of helper T lymphocytes. Nature 383, 787-793 (1996).
12. Amsen, D., Spilianakis, C. G. & Flavell, R. A. How are T(H)1 and T(H)2 effector cells made? Curr Opin Immunol 21, 153-160 (2009).
13. Motomura, Y., et al. The transcription factor E4BP4 regulates the production of IL-10 and IL-13 in CD4+ T cells. Nat Immunol 12, 450-459 (2011).
14. Karsten, C. M. & Kohl, J. The complement receptor CD46 tips the scales in T(H)1 self-control. Nat Immunol 11, 775-777 (2010).
15. Cardone, J., et al. Complement regulator CD46 temporally regulates cytokine production by conventional and unconventional T cells. Nat Immunol 11, 862-871 (2010).
16. Kemper, C., et al. Activation of human CD4+ cells with CD3 and CD46 induces a T-regulatory cell 1 phenotype. Nature 421, 388-392 (2003).
17. Ivanov, I I, et al. The orphan nuclear receptor RORgammat directs the differentiation program of proinflammatory IL-17+ T helper cells. Cell 126, 1121-1133 (2006).
18. Burchill, M. A., et al. Linked T cell receptor and cytokine signaling govern the development of the regulatory T cell repertoire. Immunity 28, 112-121 (2008).
19. O'Garra, A. Commitment factors for T helper cells. Curr Biol 10, R492-494 (2000).
20. Langrish, C. L., et al. IL-23 drives a pathogenic T cell population that induces autoimmune inflammation. J Exp Med 201, 233-240 (2005).
21. Bettelli, E., et al. Reciprocal developmental pathways for the generation of pathogenic effector TH17 and regulatory T cells. Nature 441, 235-238 (2006).
22. Veldhoen, M., Hocking, R. J., Atkins, C. J., Locksley, R. M. & Stockinger, B. TGFbeta in the context of an inflammatory cytokine milieu supports de novo differentiation of IL-17-producing T cells. Immunity 24, 179-189 (2006).
23. Mangan, P. R., et al. Transforming growth factor-beta induces development of the T(H)17 lineage. Nature 441, 231-234 (2006).
24. Korn, T., et al. IL-21 initiates an alternative pathway to induce proinflammatory T(H)17 cells. Nature 448, 484-487 (2007).
25. Laurence, A., et al. Interleukin-2 signaling via STAT5 constrains T helper 17 cell generation. Immunity 26, 371-381 (2007).
26. Liao, W., Lin, J. X., Wang, L., Li, P. & Leonard, W. J. Modulation of cytokine receptors by IL-2 broadly regulates differentiation into helper T cell lineages. Nat Immunol 12, 551-559 (2011).
27. Dong, C. & Flavell, R. A. Control of T helper cell differentiation—in search of master genes. Sci STKE 2000, pe 1 (2000).
28. Mutafova-Yambolieva, V. N., et al. Beta-nicotinamide adenine dinucleotide is an inhibitory neurotransmitter in visceral smooth muscle. Proc Natl Acad Sci U S A 104, 16359-16364 (2007).
29. Breen, L. T., Smyth, L. M., Yamboliev, I. A. & Mutafova-Yambolieva, V. N. beta-NAD is a novel nucleotide released on stimulation of nerve terminals in human urinary bladder detrusor muscle. Am J Physiol Renal Physiol 290, F486-495 (2006).
30. Hwang, S. J., et al. beta-nicotinamide adenine dinucleotide is an enteric inhibitory neurotransmitter in human and nonhuman primate colons. Gastroenterology 140, 608-617 e606 (2011).
31. Durnin, L., et al. Release, neuronal effects and removal of extracellular beta-nicotinamide adenine dinucleotide (beta-NAD(+)) in the rat brain. Eur J Neurosci 35, 423-435 (2012).
32. Smyth, L. M., Bobalova, J., Mendoza, M. G., Lew, C. & Mutafova-Yambolieva, V. N. Release of beta-nicotinamide adenine dinucleotide upon stimulation of postganglionic nerve terminals in blood vessels and urinary bladder. J Biol Chem 279, 48893-48903 (2004).
33. Penberthy, W. T. & Tsunoda, I. The importance of NAD in multiple sclerosis. Curr Pharm Des 15, 64-99 (2009).
34. Billington, R. A., et al. Emerging functions of extracellular pyridine nucleotides. Mol Med 12, 324-327 (2006).
35. Bruzzone, S., Guida, L., Zocchi, E., Franco, L. & De Flora, A. Connexin 43 hemi channels mediate Ca2+-regulated transmembrane NAD+ fluxes in intact cells. Faseb J 15, 10-12 (2001).
36. Seman, M., et al. NAD-induced T cell death: ADP-ribosylation of cell surface proteins by ART2 activates the cytolytic P2X7 purinoceptor. Immunity 19, 571-582 (2003).
37. Hubert, S., et al. Extracellular NAD+ shapes the Foxp3+ regulatory T cell compartment through the ART2-P2X7 pathway. J Exp Med 207, 2561-2568 (2010).
38. Magnone, M., et al. NAD+ levels control Ca2+ store replenishment and mitogen-induced increase of cytosolic Ca2+ by Cyclic ADP-ribose-dependent TRPM2 channel gating in human T lymphocytes. J Biol Chem 287, 21067-21081 (2012).
39. Axtell, R. C., et al. T helper type 1 and 17 cells determine efficacy of interferon-beta in multiple sclerosis and experimental encephalomyelitis. Nat Med 16, 406-412 (2010).
40. Kohm, A. P., Carpentier, P. A., Anger, H. A. & Miller, S. D. Cutting edge: CD4+CD25+ regulatory T cells suppress antigen-specific autoreactive immune responses and central nervous system inflammation during active experimental autoimmune encephalomyelitis. J Immunol 169, 4712-4716 (2002).
41. Zozulya, A. L. & Wiendl, H. The role of regulatory T cells in multiple sclerosis. Nat Clin Pract Neurol 4, 384-398 (2008).
42. Bettelli, E., et al. IL-10 is critical in the regulation of autoimmune encephalomyelitis as demonstrated by studies of IL-10- and IL-4-deficient and transgenic mice J Immunol 161, 3299-3306 (1998).
43. Kaneko, S., et al. Protecting axonal degeneration by increasing nicotinamide adenine dinucleotide levels in experimental autoimmune encephalomyelitis models. J Neurosci 26, 9794-9804 (2006).
44. Zelenika, D., et al. Regulatory T cells overexpress a subset of Th2 gene transcripts. J Immunol 168, 1069-1079 (2002).
45. Nowak, E. C., et al. Tryptophan hydroxylase-1 regulates immune tolerance and inflammation. J Exp Med 209, 2127-2135 (2012).
46. Saraiva, M., et al. Interleukin-10 production by Th1 cells requires interleukin-12-induced STAT4 transcription factor and ERK MAP kinase activation by high antigen dose. Immunity 31, 209-219 (2009).
47. Wang, Y. H., et al. A novel subset of CD4(+) T(H)2 memory/effector cells that produce inflammatory IL-17 cytokine and promote the exacerbation of chronic allergic asthma. J Exp Med 207, 2479-2491 (2010).
48. Berger, U. V., Grzanna, R. & Molliver, M. E. Depletion of serotonins using p-chlorophenylalanine (PCPA) and reserpine protects against the neurotoxic effects of p-chloroamphetamine (PCA) in the brain. Exp Neurol 103, 111-115 (1989).
49. Cote, F., et al. Disruption of the nonneuronal tph1 gene demonstrates the importance of peripheral serotonin in cardiac function. Proc Natl Acad Sci USA 100, 13525-13530 (2003).
50. Lee, Y. et al. Induction and molecular signature of pathogenic Th17 cells. Nat Immunol 13, 991-999 (2012).
51. Asadullah, K., Sterry, W. & Volk, H. D. Interleukin-10 therapy—review of a new approach. Pharmacol Rev 55, 241-269 (2003).
52. Pennline, K. J., Roque-Gaffney, E. & Monahan, M. Recombinant human IL-10 prevents the onset of diabetes in the nonobese diabetic mouse. Clin Immunol Immunopathol 71, 169-175 (1994).

### Example 2

### NAD⁺ Alone Converts nTregs into Th17 Cells and Prolongs Allograft Survival

CD4⁺ CD25⁺ Foxp3⁻ natural regulatory T cells (nTregs) play a critical role in the maintenance of immune tolerance and T cell homeostasis¹⁻². It is well established that nTregs inhibit autoimmunity and inflammation through multiple mechanisms including the production of IL-10, a potent immunomodulatory cytokine or, alternatively, through TGFβ known to suppress IFNγ and Tbx21 production, a master regulator of T helper 1 (Th1) cells³. In addition, nTregs have the capacity to regulate the immune response by killing CD4⁺ T effector cells through the secretion of granzyme A and B as well as perforin³. Another critical immunoregulatory capacity of nTregs is linked to their capacity to induce apoptosis of T effector cells by IL-2 deprivation³. Tregs were first described in 1995 by Sakaguchi et al. Although this lineage has been recognized as CD4⁺ T cell type, CD4⁺CD25⁻ FoxP3⁺ nTregs constitute a distinct thymus-derived T-cell lineage³. An additional type of Treg has been characterized and termed induced regulatory T cells (iTregs) which originate in the periphery upon T cell receptor (TCR) stimulation in the presence of TGF-β². Both, nTregs and iTregs express the transcription factor FoxP3 constitutively which is the master regulator required for Treg development and their suppressive function².

Although many studies have reported Tregs, in particular nTregs, as a stable lineage, recent observations have been challenging this concept⁴⁻⁵. It has been shown that using an agonist antibody to the T cell Ig mucin-1 or nitric oxide result in the loss of Foxp3 expression in nTregs⁶. Furthermore, in presence of IL-6 nTregs have been shown to lose Foxp3 expression following TCR engagement³. Thus, increasing evidence indicates that Tregs can lose Foxp3, thus acquiring diverse T effector/helper functions under certain inflammatory conditions⁷. nTregs have been shown to have the capacity to convert into T helper17 (Th17) cells in presence of TGFβ and/or IL-6⁸⁻⁹. Voo et al. reported in human a Treg population that expresses Foxp3 and produces IL-17 while retaining its suppressive function¹⁰. Th17 is a CD4^{|} T cell subset distinct from Th1 and Th2 that secrete IL-17A, IL-17F, IL-22 and require the transcription factor RORγt¹¹⁻¹⁴. Th17 cells are involved in host defenses against bacteria and fungi¹⁵. Elevated levels of IL-17A cytokine have been associated with autoimmune diseases such as rheumatoid arthritis, asthma, systemic lupus erythematosus, scleritis and allograft rejection¹⁵. Numerous studies have shown that TGFβ, IL-6, IL-21 and IL-23 are critical for Th17 differentiation and proliferation¹⁵. In contrast to nTregs, IL-2 has been shown to inhibit Th17 development¹⁶⁻¹⁷. Almost three decades ago when first Th1 and Th2 subsets were described it is considered that the major determinant of the differentiated state of CD4⁺ T cells in vitro is the cytokine environment¹⁸⁻²⁰.

As demonstrated herein, nicotinamide adenine dinucleotide (NAD⁺), a cofactor absorbed from nutrients or released during inflammation or produced under physiological conditions by many different cell types such as epithelial cells, neurons or fibroblasts²¹⁻²⁸ regulates, in vitro, human and mouse nTregs conversion into IL-17⁺FoxP3⁺ cells in presence of IL-2 and without exogenous addition of TGFβ, IL-6, IL21 and IL-23 following TCR engagement. Moreover, nTregs differentiated into IL-17⁺FoxP3⁺ cells under those conditions lost their ability to produce IL-10, a hallmark cytokine of Tregs. Using a fully allogeneic mouse skin transplant model in which nTregs and Th17 cells have been shown to play a critical role in allograft survival, it was found that treatment with NAD⁺ resulted systemically in a significant decrease of nTreg frequency and an increased frequency of IL-17A⁺ producing cells. Although nTregs have been shown to promote allograft survival and Th17 cells to enhance allograft rejection, NAD⁺ treatment resulted in a dramatic increased allograft survival. Strikingly, NAD⁺ regulated the systemic alloimmune response and promoted allograft survival through the immunosuppressive IL-10 cytokine.

NAD⁺ Promotes nTreg Conversion into Th17 Cells and Their Proliferation in Vitro in the Absence of TGFβ, IL6, IL-23 and in the Presence of IL-2

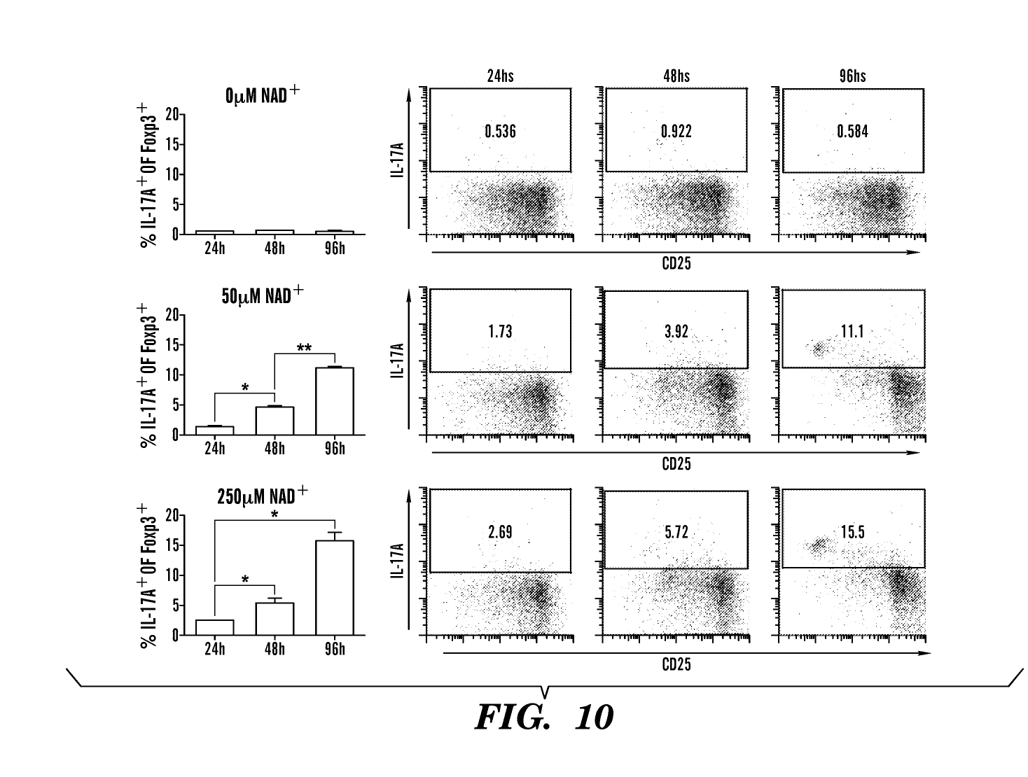

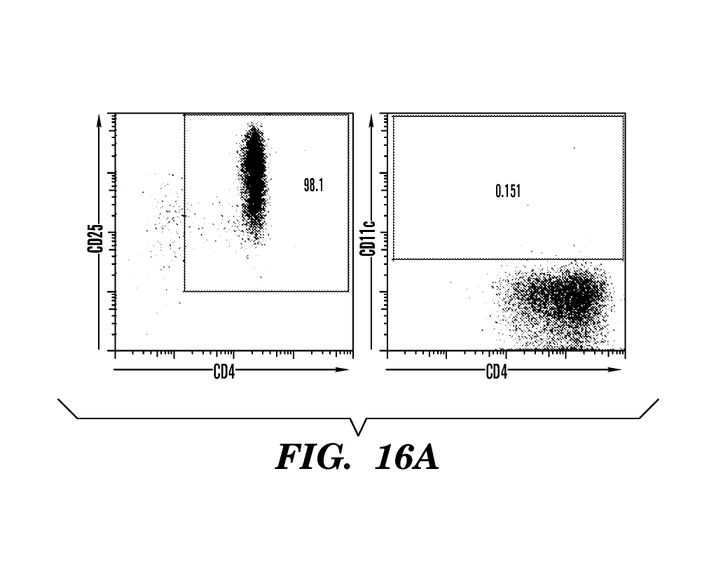

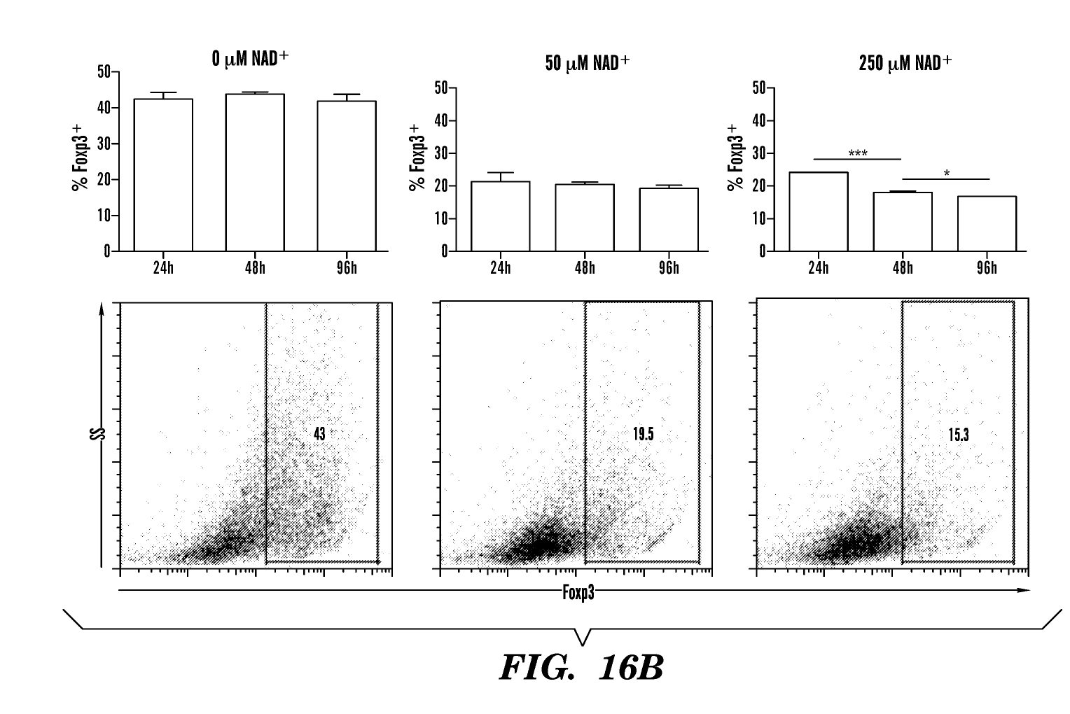

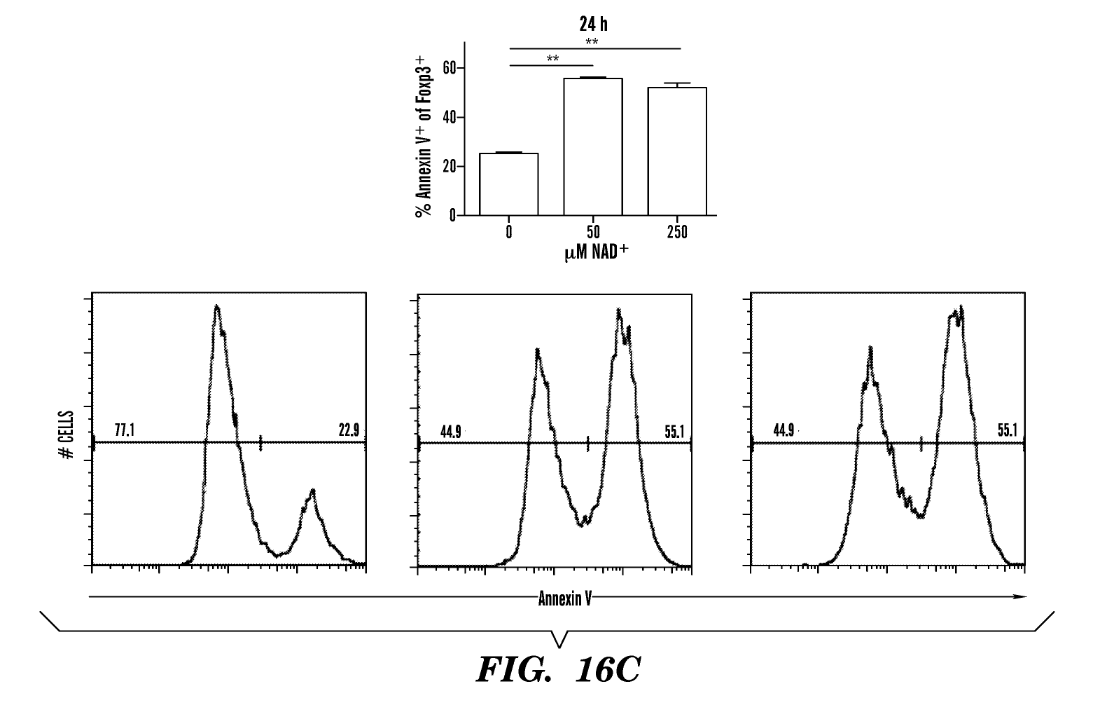

Recent evidence has challenged the notion that Tregs represent a stable lineage⁷. It has been proposed that under specific inflammatory conditions Tregs may lose FoxP3 expression and acquire effector functions⁶⁻⁷. Numerous studies have shown that Tregs can convert into Th17 cells⁸⁻¹⁰. TGFβ, IL-6, IL-21 are critical for Th17 differentiation in vitro after TCR engagement while IL-23 promotes Th17 proliferation¹⁵. In contrast, addition of IL-2 has been shown to prevent Th17 development¹⁶⁻¹⁷. A recent study has shown that ATP can promote Treg conversion into Th17 cells in presence of IL-6 via purinergic receptors, in particular P2RX7²⁹. It is well established that ATP and NAD⁺ can both activate P2XR7³⁰⁻³¹. more importantly, it was shown that P2XR7 activation by NAD⁺ required lower concentration than ATP³⁰. In a recent study, Hubert et al. demonstrated that NAD⁺, a cofactor released during inflammation can regulate T cell homeostasis through selective depletion of nTreg³². Thus, it was tested whether NAD⁺ induces the loss of Foxp3 expression and whether this may subsequently confer an effector function to nTregs. CD4⁺CD25⁺ nTregs were isolated from spleens of DBA mice with high purity (FIG. 16A) and cultured at different time points under varying concentrations of NAD⁺. Of note, nTregs were cultured initially without TGFβ, IL-6 and in presence of IL-2 (50 ng/ml). Consistent with previous studies³², increased NAD⁺ concentrations reduced frequencies of CD4⁺CD25⁺FoxP3⁺ cells (FIG. 16B) as a result of apoptosis (FIG. 16C). However, increasing concentrations of NAD⁺ were associated with higher frequencies of CD4⁺CD25⁻IL-17A⁺Foxp3⁺ (FIG. 10). After 96 hrs of culture and in presence of 250 μM of NAD⁺ more than 16% of the cells were able to produce IL-17A in vitro (FIG. 10).

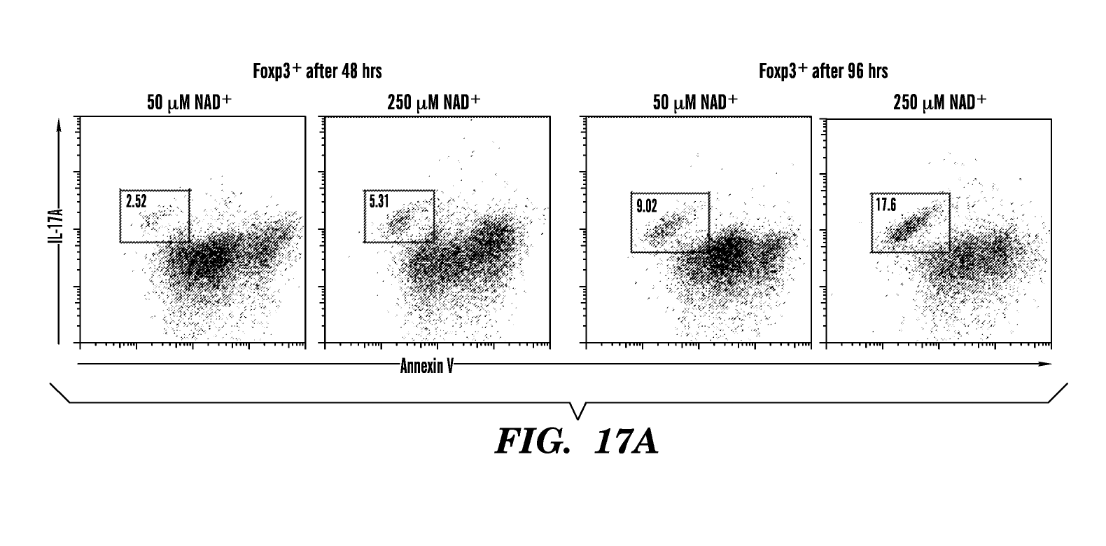

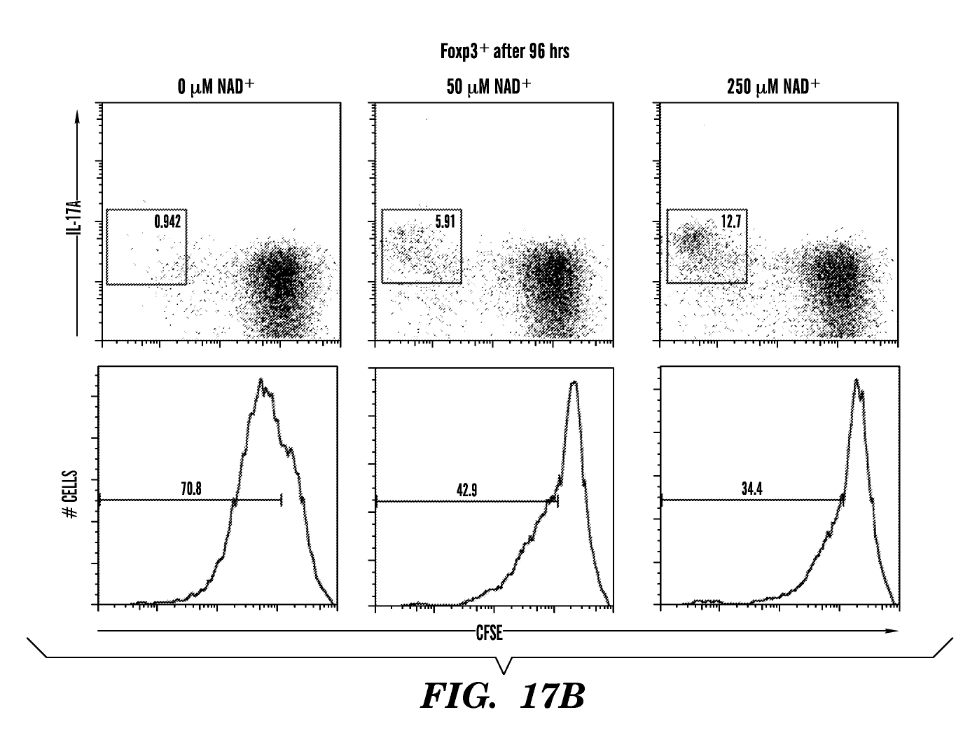

Next, it was tested whether the increasing number of CD4⁺CD25⁻IL-17A⁺Foxp3⁻ cells was the result of fewer nTregs subsequent to apoptosis caused by NAD⁺. Therefore, nTregs were cultured in the presence of increasing concentrations of NAD⁺ and apoptosis was assessed after 48 and 96 hrs with annexin V. Although CD4⁺CD25⁻IL-17A⁺Foxp3⁺ cells did not become apoptotic (FIG. 17A); the results indicated that CD4⁺CD25⁻IL-17A⁺Foxp3⁺ cells were able to proliferate in presence of NAD⁺ in a dose dependent manner (FIG. 17B). Taken together, these results demonstrated that NAD⁺ induced in vitro, nTreg conversion into IL-17A producing cells in the absence of exogenous TGFβ and IL-6, and in the presence of IL-2. Moreover, NAD⁺ enhanced IL-17A producing cell proliferation in the absence of exogenous IL-23.

NAD⁺ Promotes nTreg Conversion into Th17 Cells Specifically

It was next investigated whether NAD⁺ was able to promote nTreg conversion specifically to Th17 cells. nTregs were cultured in presence of increasing NAD⁻ concentrations and mRNA and cytokine levels that are specific to Th1, Th2, Tregs and Th17 were measured by real-time PCR and ELISA.

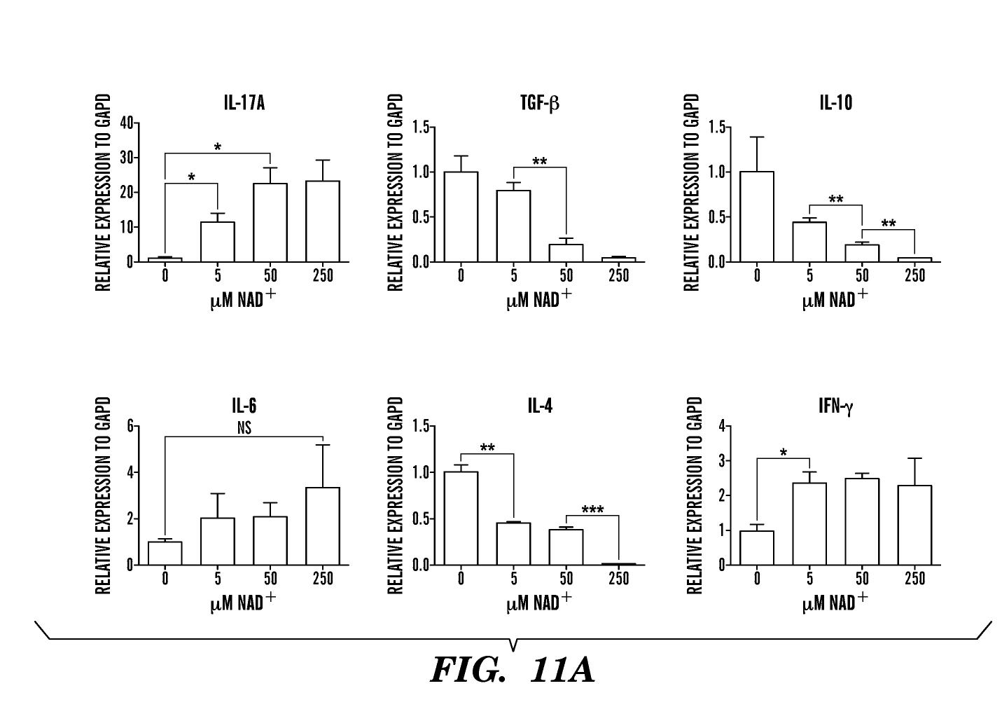

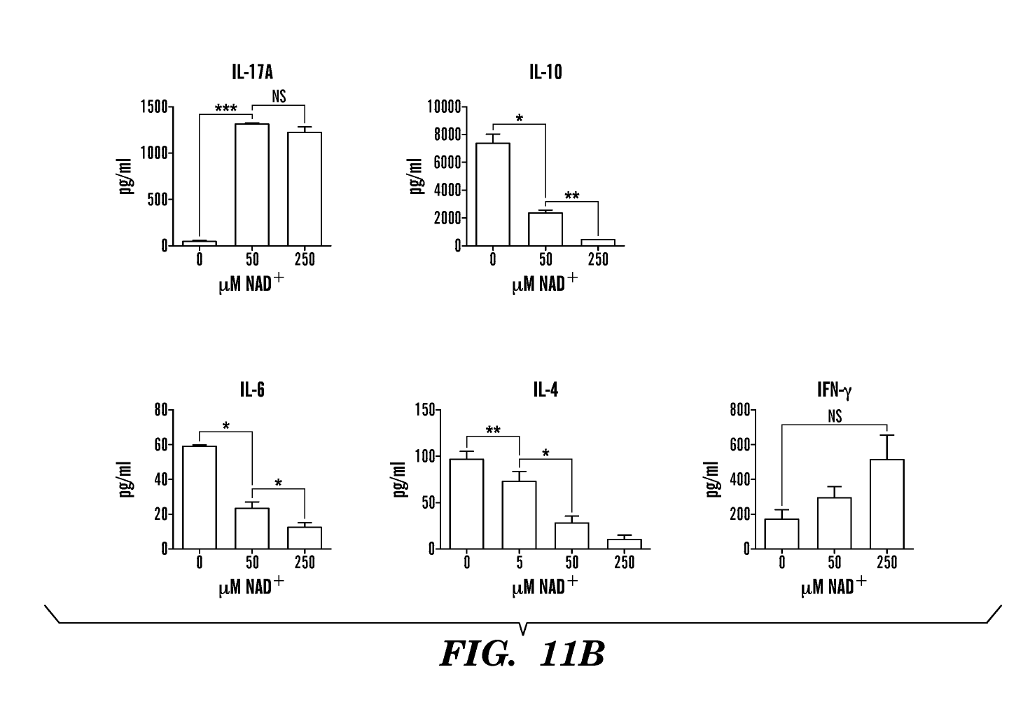

When nTregs were cultured in presence of NAD⁺, IL-17A mRNA and protein levels increased in a dose dependent manner (FIG. 11A and 11B). At the same time, mRNA and cytokine levels, such as IL-10 and TGFβ typically produced by nTregs decreased dramatically (FIG. 11A and 11B). Similarly, mRNA and cytokine levels of IFNγ and IL-4, cytokines typically produced by Th1 and Th2 subsets respectively, decreased in a dose dependent manner in the presence of NAD⁺ (FIG. 11A and 11B). Thus, NAD⁺ promotes specifically a Th17 cytokine expression and secretion.

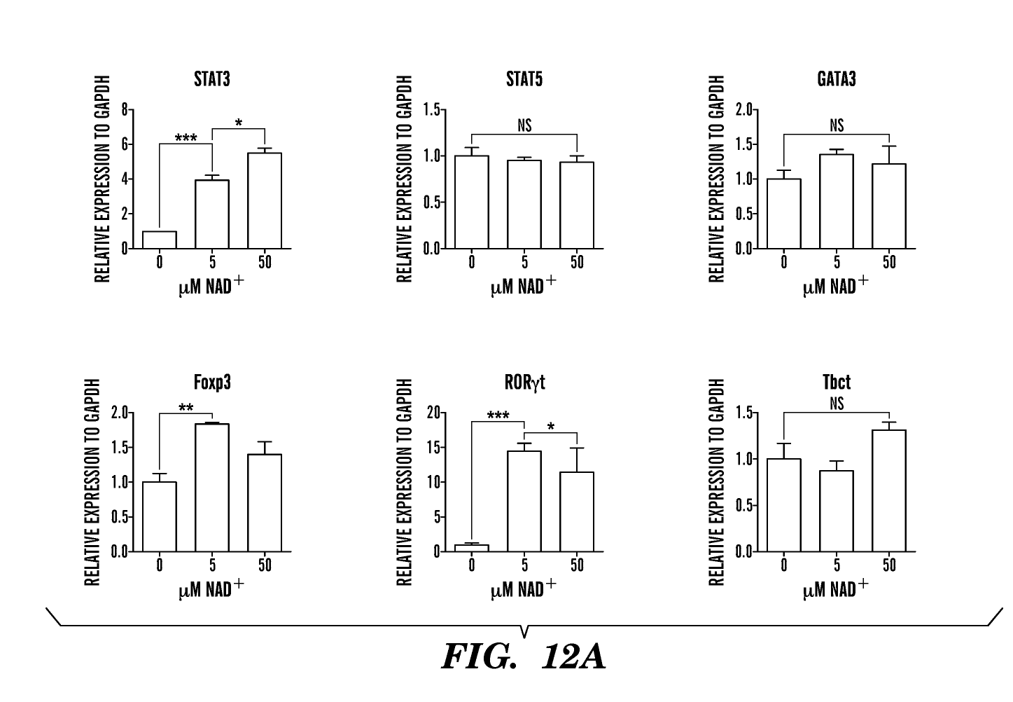

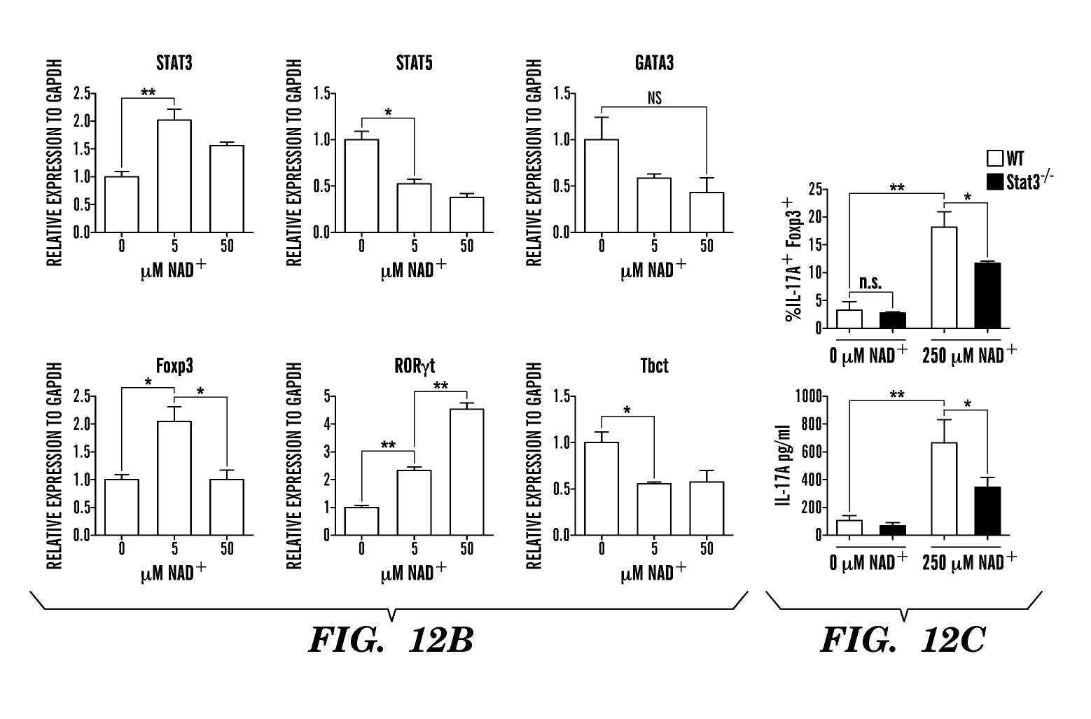

It is well established that Tbx-21 (T-bet) is critical for Th1 commitment while GATA-3 and STAT5 regulate Th2 and Treg differentiation and maintenance. Moreover, it has been shown that STAT3 and RORγt are essential for Th17 differentiation while FoxP3 is required for Treg development. Thus, transcription factors that are required for Th1, Th2, Th17 and Treg development were investigated. Isolated nTregs were cultured in presence of increasing NAD⁺ concentrations and mRNA levels for Tbx21, GATA-3, FoxP3, STAT3 and STAT5 were measured 24 (FIG. 12A) and 96 hrs (FIG. 12B). After 24 hrs of culture STAT3 (>10 fold), RORγt (>10 fold) and Foxp3 had significantly increased while the expression level of Tbx21, GATA3 and STAT5 remained unchanged (FIG. 12A). Of note, levels of STAT3, FoxP3 and RORγt remained significantly increased after 4 days of culture with NAD⁻ while mRNA levels of Tbx21, GATA-3 STAT5 had decreased significantly (FIG. 12B). It has been shown that STAT3 can attenuate FoxP3 expression and promote Th17 development³³ and the results indicated that NAD⁻ enhanced STAT3 expression levels. Thus, it was tested whether NAD⁺ promotes the conversion of nTreg into Th17 cells through the transcription factor STAT3. nTregs were isolated from STAT3^{-/-} and wild type (WT) mice and cultured with or without NAD⁺. IL-17A⁺ cells and IL-17A cytokine production were quantified 96 hrs later by flow cytometry and ELISA, respectively. The results indicated that nTregs from STAT3^{-/-} mice had a significant decreased, but not complete, frequency of IL-17A⁺ cells and a reduced IL-17A cytokine production (FIG. 12C). Taken together, these results indicate that NAD⁺ induces nTreg conversion into Th17 cells in part through the transcription factor STAT3.

NAD⁺ Promotes the Conversion of Human nTreg into IL-17-A Producing Cells

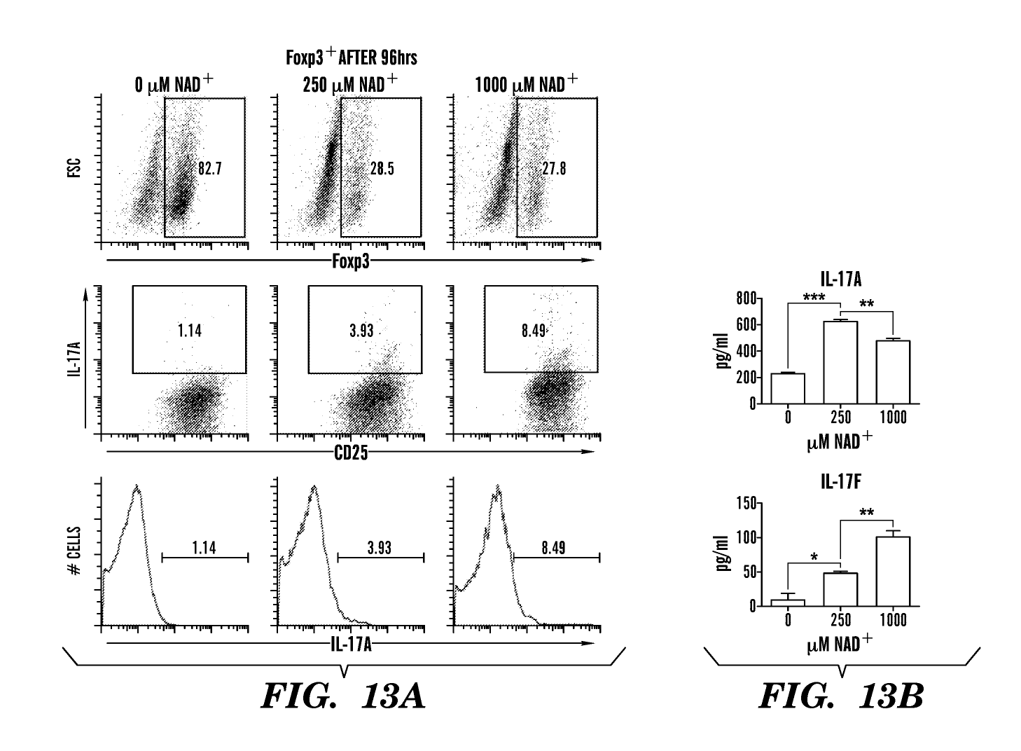

Several discrepancies have been reported for mechanisms promoting Th17 cell development in mice and human¹⁵. In mice, Th17 differentiation has been shown to require TGFβ, IL-6 and IL-21 cytokines while IL-23 was critical to sustain Th17 cells. In addition, it has been shown that IL-2 inhibits Th17 development¹⁶. In contrast, human Th17 differentiation requires IL-1β and IL-6 and can be inhibited by TFG-β and IL-2³⁴. However, latter reports indicated that TGF-β and IL-2 play a more critical role in Th17 commitment³⁵⁻³⁶ . To make matters more confusing, an additional study showed that differentiation of Th17 cells from human naive conventional CD4⁺ T cells required TGF-β and IL-21 but not TGF-β and IL-6³⁷. Moreover, it was suggested that several inflammatory cytokines including IL-1β, IL-6 and IL-23 were all required acting in a synergistic fashion³⁸. Therefore, it was tested whether NAD⁺ was a universal molecule that was able to enhance nTreg conversion into Th17 cells in human and mice as well. Human nTregs were isolated and cultured in presence of increasing NAD⁻ concentrations and IL-2. FIGS. 13A and 13B showed that NAD⁺ increased the number of IL-17A⁺ cells (FIG. 13A) and to enhance IL-17A cytokine production (FIG. 13B). Of note, human nTregs conversion into IL-17A⁺ producing cells remained CD4⁺CD25⁺. Taken together, these results indicate that NAD⁺ is able to convert human CD4⁺CD25⁺FoxP3⁺ nTregs into IL-17A producing cells.

NAD^{|} Promotes nTreg Conversion into Th17 Cells Through Purinergic Receptors

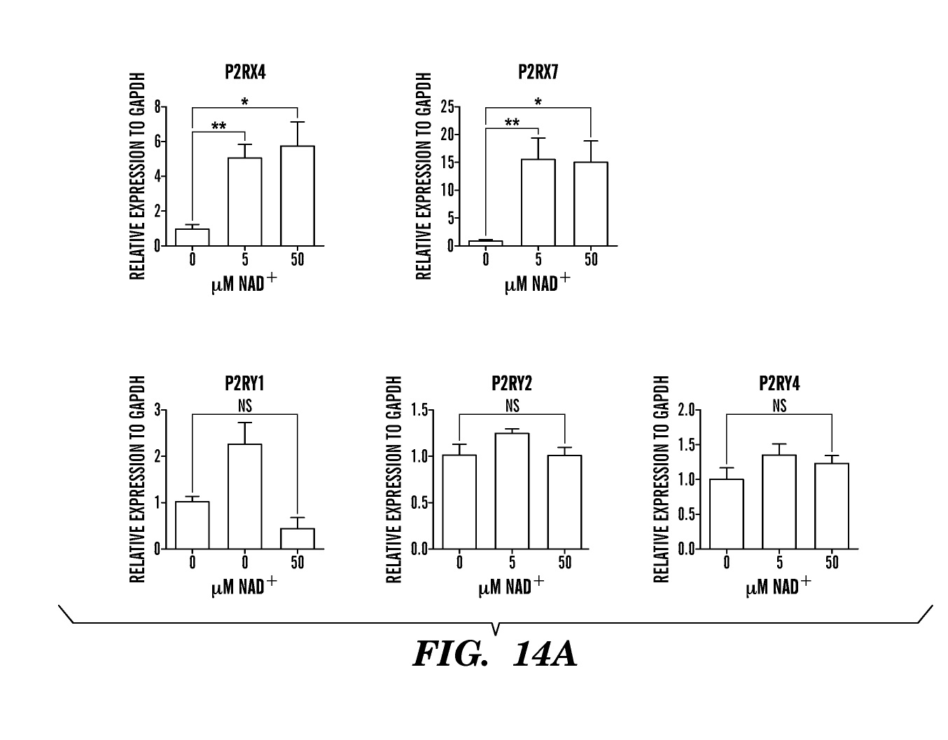

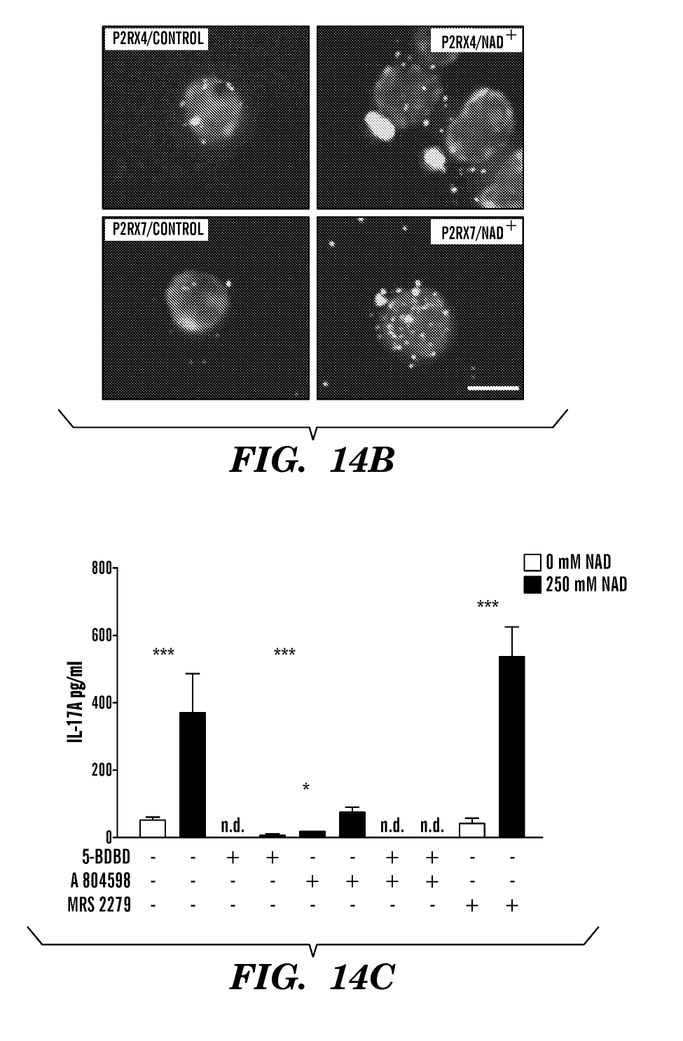

Several purinergic receptors including P2RX4, 7 and P2RY1, 2, 4 have been reported to regulate T cell activation and function³⁹. More importantly, it has recently been shown that purinergic activation, in particular P2RX7, by ATP promotes the conversion of Tregs into Th17 cells in presence of IL-6²⁹. Furthermore, it was shown that both ATP as well as NAD⁺ can activate purinergic receptors via ART2.2 pathway and that P2RX7 had much higher sensitivity to low concentrations of NAD when compared to ATP³⁰⁻³¹. Thus, the inventors tested whether the capacity of NAD⁺ to promote nTregs conversion into Th17 cells was mediated through purinergic signaling activation. nTregs was stimulated with anti-CD3/CD28 in the presence or not of NAD⁺ and mRNA levels for P2RX4, 7 and P2RY1, 2, 4 were measured 24 hrs later by real-time PCR. Results indicated an up-regulation of P2RX4 (>4 fold) and P2RX7 (>15 fold) expression levels while P2RY1, 2 and 4 levels remained unchanged (FIG. 14A). Furthermore, the immunofluorescence results showed that NAD⁺ increased the expression of P2RX4 and P2RX7 receptors on the cell surface resulting in their clustering (FIG. 14B). Virtual reality quick time movies of 3-D reconstructed control and NAD⁻ treated T cells stained for P2RX4 and P2RX7 have been demonstrated. Next, it was investigated whether inhibition of P2RX4 and P2RX7 with the use of highly selective antagonists was able to block nTreg conversion into Th17 by NAD⁻. When nTregs were cultured in presence of 5-(3-Bromophenyl)-1,3-dihydro-2H-benzofuro[3,2-e]-1,4-diazepin-2-one (5-BDBD) and/or A804598, two selective antagonists of P2RX4⁴⁰ and P2RX7⁴¹⁻⁴², respectively, a dramatic reduced production of IL-17A cytokine was observed that was complete when both selective antagonists were added (FIG . 14C). Of note, selective inhibition of P2RX4 resulted in a more robust blockage of IL-17 production when compared to P2RX7 (FIG. 13C). In contrast, using a selective antagonist (MRS 2279) for P2RY1⁴³ did not change IL-17A⁺ production by NAD⁺ (FIG. 14C). Collectively, these results indicate that NAD⁺ ability to convert nTregs into Th17 cells is mediated through purinergic signaling.

### NAD⁺ Plays a Critical Role in Immune Tolerance Through a Systemic Increase of IL-10 Cytokine

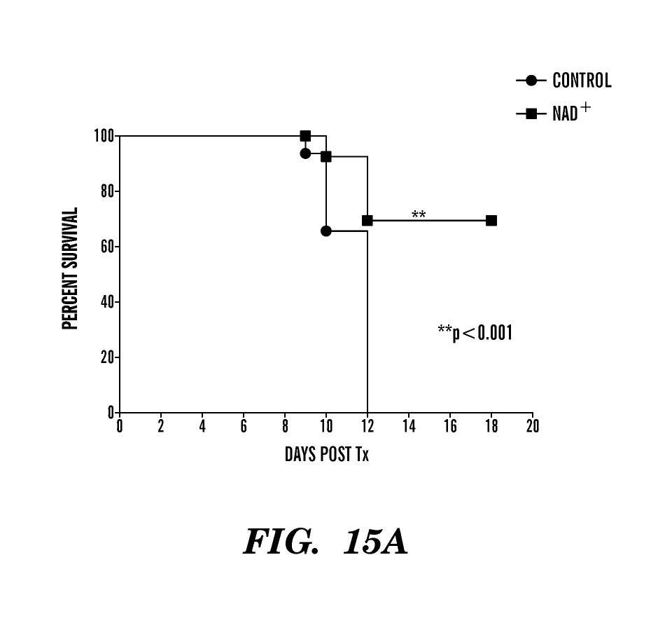

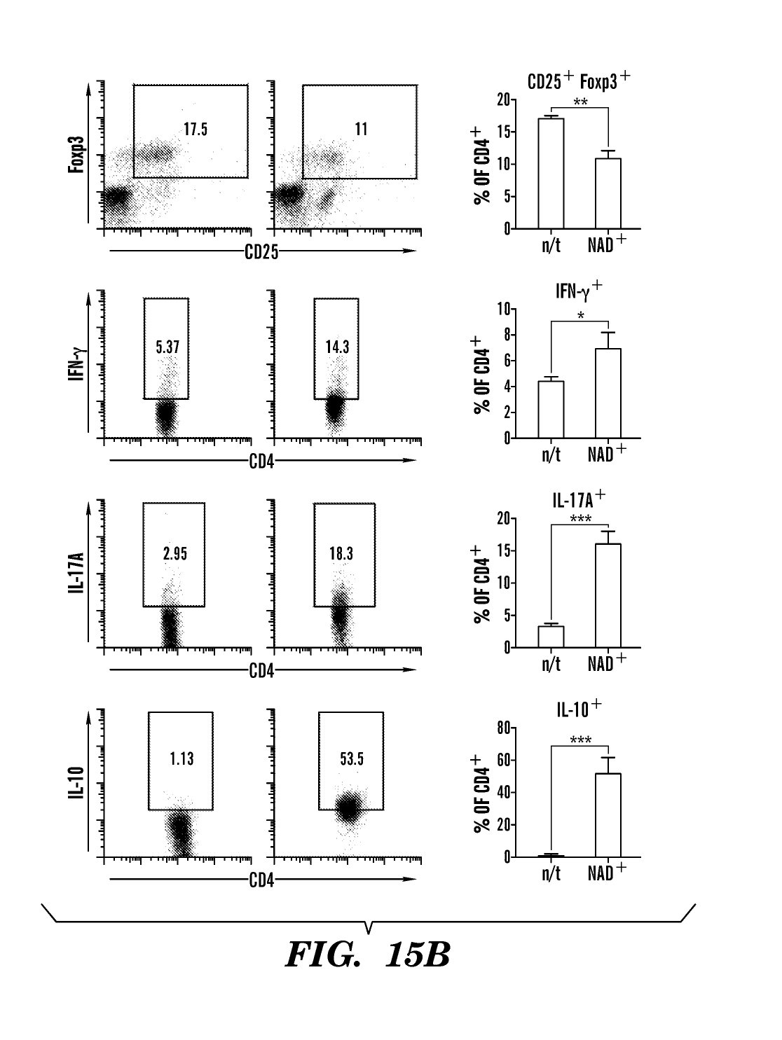

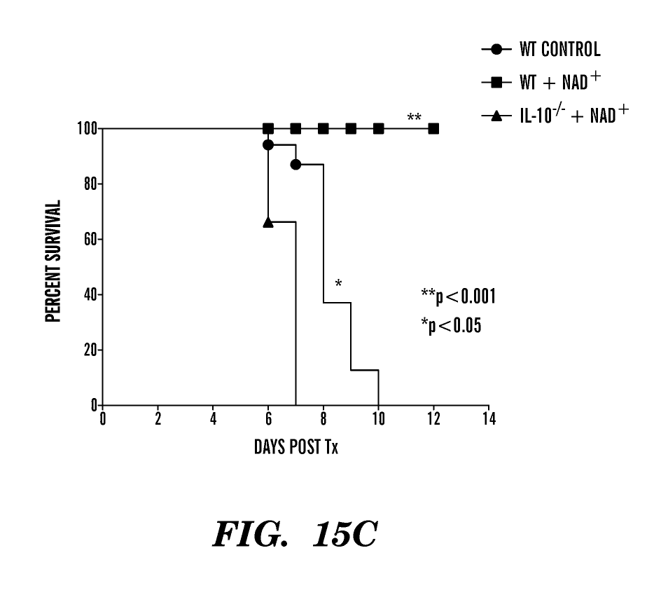

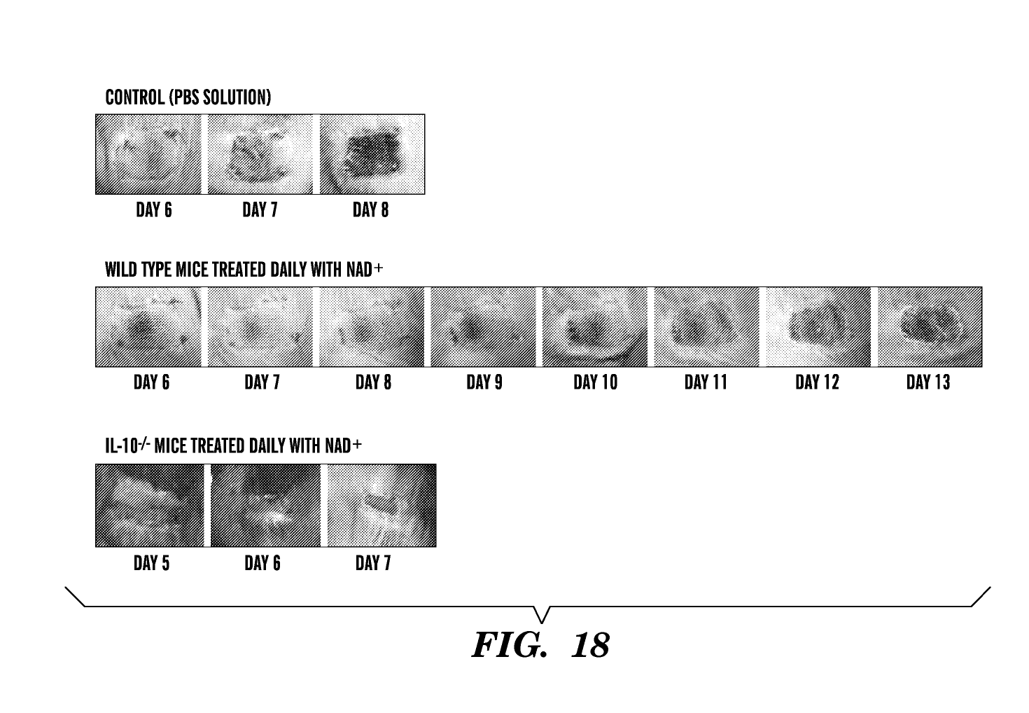

In vitro findings showed that NAD^{|} promoted the conversion of nTregs into Th17 cells. nTregs play an important role in immune tolerance while IL-17A has been shown to be a robust inflammatory cytokine involved in many diseases¹⁵. More importantly, in transplantation nTregs promote allograft survival while Th17 cells enhance allograft rejection⁴⁴⁻⁴⁵. Thus, to study, in vivo, the impact of NAD⁺ on nTregs and Th17 cells a transplant mouse model was used. Fully MHC-mismatched C57BL/6 (H2^{b}) tail skin allografts were transplanted onto DBA/2 (H2^{d}) mice that received daily intraperitoneally a solution containing NAD⁺ or a placebo solution (PBS) and graft survival was monitored. In vitro data indicated that NAD⁺ promoted nTreg conversion into Th17 cells. Therefore, NAD⁺ treatment was expected to promote allograft rejection. Surprisingly, recipient mice treated daily with NAD⁺ exhibited a dramatic allograft survival when compared to the control group. Mean survival time of allografts in untreated recipient DBA mice was 10 days (FIG. 14A) while 66% of the allografts in NAD⁺ treated recipient animal survived more than 18 days (FIG. 15A) and no visual sign of rejection 13 days after transplantation was observed (FIG. 18). Next, it was investigated whether nTreg conversion into Th17 cells observed in vitro by NAD⁺ was observed in vivo as well. Recipient mice were evaluated by day 8 and the frequency of nTregs CD4⁺CD25⁺Foxp3⁺ and CD4⁺IL17-A⁺ cells was assessed (FIG. 14B). When compared to the control group of mice that received a placebo solution, recipient mice that were treated with NAD⁺ had a reduced frequency of CD4⁺CD25⁺Foxp3⁺ nTregs and increased frequencies of CD4⁺IL-17A⁺ cells (FIG. 14B) that was consistent with the in vitro results. Next, mechanisms by which NAD⁺ could promote nTreg conversion into Th17 cells and allograft survival at the same time, e.g., two paradoxes, were assessed. Strikingly, when compared to the control group, more than 60% of CD4⁺ T cells of NAD⁺ treated mice were IL-10⁺ producing cells (FIG. 14B). It is known that IL-10 is a robust immunosuppressive cytokine⁴⁶. Therefore, it was tested whether the allograft survival observed in recipient mice treated with NAD^{|} was the results of a systemic increase of IL-10 production. Thus, WT and IL-10^{-/-} mice (on a C57BL6/ background) received a BDA skin transplant and were treated with NAD^{|}. The results shown in FIG. 14C and FIG. 18 indicated that treatment with NAD⁺ in IL-10^{-/-} mice abolished the allograft survival previously observed with NAD⁺ treatment and was significantly reduced when compared to the WT non treated mice as well. Collectively, these results indicate that NAD⁺ reduces nTregs systemically while it promotes IL-17A. More importantly, these results demonstrate that NAD⁺ induces immune tolerance independently from nTregs through a massive systemic production of IL-10 cytokine.

### Discussion

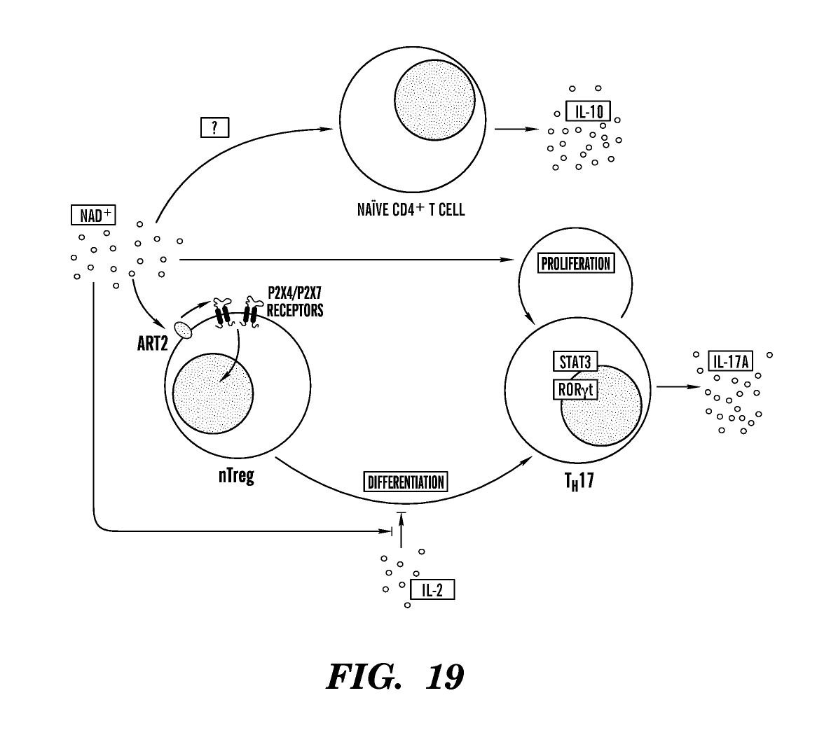

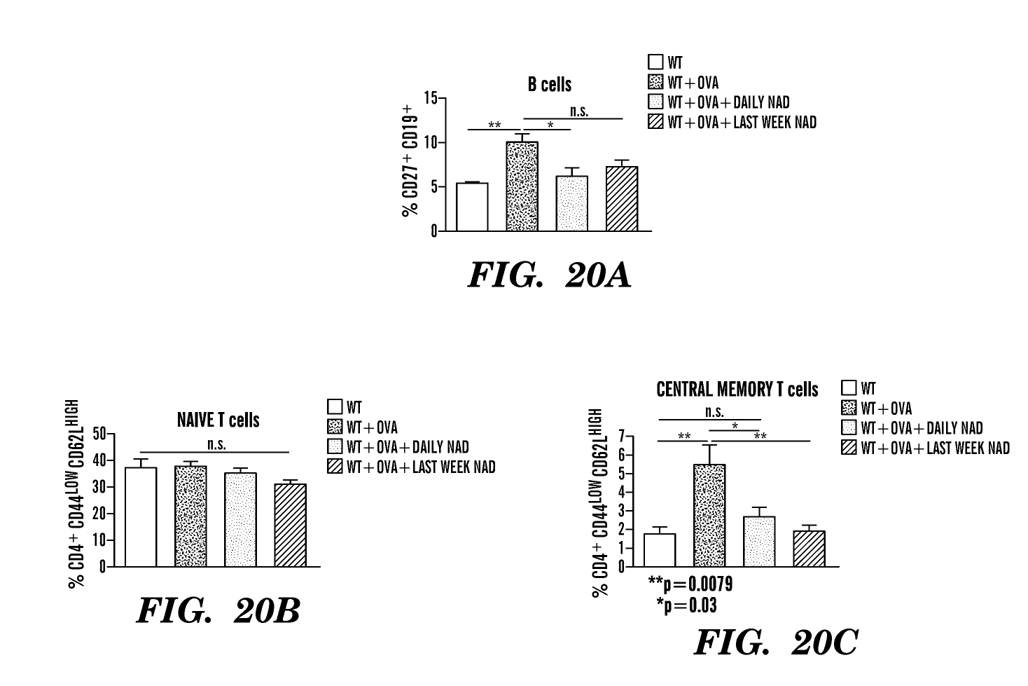

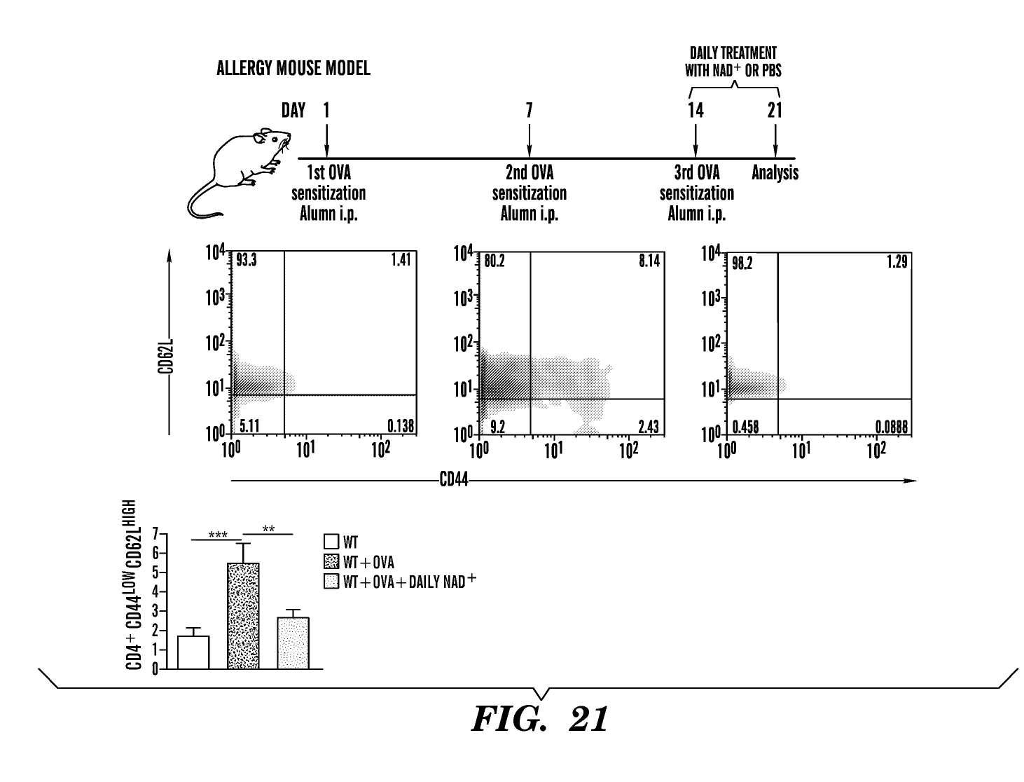

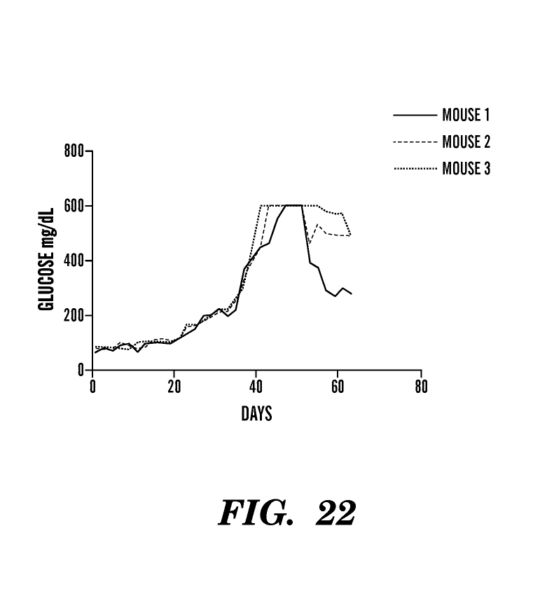

The inventors have uncovered a novel differentiation pathway that converts mouse and human nTregs into Th17 cells using a natural molecule found in the body that does not require the addition of exogenous cytokines such as TGFβ, IL-6 or IL-21 (FIG. 19). More importantly, NAD⁺ was able to induce Th17 differentiation even in the presence of high dose of IL-2, a cytokine known to inhibit Th17 commitment (FIG. 19). Moreover, NAD⁺ had the capacity to induce Th17 proliferation in the absence of IL-23, a critical cytokine for the maintenance of Th17 cells (FIG. 19). Th17 development was specific since NAD⁺ was able to up-regulate the expression of STAT-3 and the transcription factor RORγt a master regulator of Th17 development (FIG. 19). Moreover, NAD⁺ downregulated T-bet and GATA-3, two transcription factors that are specific for Th1 and Th2 subsets, respectively. In addition, mRNA and protein levels of IL-10 and TGFβ, two cytokines that are produced by nTregs, were reduced in a dose dependent manner indicating that NAD⁻ was able to repress nTregs maintenance. Consistent with a previous study³², NAD⁺ induced nTregs apoptosis and it cannot be ruled out that this phenomenon may take place because of the heterogeneity within nTregs and a difference in the expression pattern of markers, in particular CD25, the alpha chain of the IL-2 receptor. In addition, using nTregs that are deficient for the transcription factor STAT3^{-/-}, reduced the conversion of CD4⁺CD25⁺FoxP3⁺ into IL-17A⁺producing cells, in part, indicating that other transcription factor(s) may be involved in this process.

It has been shown that similarly to ATP, NAD⁺ activates P2RX7 at very low concentrations³⁰⁻³¹. In contrast to NAD⁺, ATP requires the presence of IL-6 cytokine²⁹ to convert Tregs into IL-17A producing cells indicating that the higher affinity of NAD⁻ to purinergic receptors may induce a more robust response and/or activate additional signaling pathways. In addition, P2RX4 and to a lower extent P2RX7 blockade reduced nTreg conversion into IL-17 producing cells, indicating that these receptors may play a different role in NAD⁺ signaling mechanisms and nTreg conversion.

The major determinant for T cell differentiation or conversion, in vitro, is the specific cytokine milieu during TCR activation. For instance, Th1 and Th2 differentiation requires IL-12 and IL-4 cytokines, respectively. However, in vivo studies have shown that Th1 and Th2 differentiation takes place in the absence of IL-12 and IL-4 suggesting alternative pathways^{6,47-48}. Here, the inventors showed that NAD⁺ was able to convert nTregs into CD4⁺IL-17A⁺ producing cells in vitro and in vivo as well. Using a skin transplant mouse model, treatment with NAD⁺ was shown to reduce the frequency of nTregs and increase Th17 responses. nTregs play a key role in immunosuppression and T cell homeostasis and have been shown to promote allograft survival. In contrast, IL-17A is a robust pro-inflammatory cytokine that enhances allograft rejection. Surprisingly, recipient animals that were treated daily with NAD⁺ had a dramatic increased allograft survival (FIG. 15A and FIG. 18). Strikingly, the inventors found that approximately 60% of CD4⁺ T cells express the robust immunosuppressive cytokine IL-10. When IL10^{-/-} mice were treated with NAD⁻ allograft survival was shorter than the treated group of WT mice, indicating that the increased allograft survival in NAD⁺ treated animals resulted from the systemic increase of IL-10. Consistent with previous studies, allograft survival in IL10^{-/-} mice was significantly reduced when compared to the WT group that was not treated, indicating that IL-10 promotes allograft survival⁴⁶. More importantly, the decreased frequency of nTregs in vivo and the reduced IL-10 expression and protein levels observed in vitro after NAD^{|} treatment indicate that the increased frequency of CD4^{|}IL-10^{|} producing cells may not originate from nTregs but conventional CD4^{|} T cells.

### Materials and Methods

### Animals

Six to eight weeks old C57BL/6 (B6, H2^{b}) and DBA/2 (H2^{d}) mice were purchased from Charles River Laboratories (Wilmington, Mass.). Stat3^{-/-} (B6.129S1-Stat3^{tm1Xyfu}/J) and IL-10^{-/-} (B6.129P2-IL10^{tmCgn}/J) mice were purchased from Jackson Laboratory Animal use and care was in accordance with institutional and National Institutes of Health guidelines.

### Isolation of Regulatory T Cells

Single-cell leukocyte suspensions were obtained from spleens. Depletion of non-CD4⁺ T cells was done using biotin-conjugated monoclonal anti-mouse antibodies against CD8α, CD11b, CD45R, CD49b, Ter-119 and anti-biotin magnetic beads (MILTENYI BIOTEC, Bergisch Gladbach, Germany). Cells were further sorted using α-CD25-PE and α-PE magnetic beads (MILTENYI BIOTEC). Purities of regulatory T cells after isolation were >98%.

For isolation of human Treg cells, peripheral blood mononuclear cells were obtained from young healthy volunteers using density gradient centrifugation with Ficoll-Paque (STEMCELL TECHNOLOGIES, Vancouver, BC, Canada). Non-CD4⁺ T cells were then depleted using biotin-conjugated monoclonal anti-human antibodies against CD8, CD14, CD15, CD16, CD19, CD36, CD56, CD123, TCRγ/δ, CD235a (Glycophorin A) and anti-biotin magnetic beads (Miltenyi Biotec). CD4⁺ CD25⁺ cells were then obtained using α-CD25 magnetic beads (Miltenyi Biotec). Purities of T reg cells were >98%.

### Functional In-Vitro Treg Cell Assays

Isolated murine Treg cells were cultivated in 48-well flat bottom plates (2.5×10⁴ cells per well) in 0.5 ml of complete media) in presence of 10 μg/ml plate-bound anti-mouse α-CD3 (17A2) and 2 μg/ml soluble α-CD28 (37.51) in addition to 50 μg/ml recombinant mouse IL-2 (all EBIOSCIENCE). NAD⁺ (Sigma-Aldrich) was added at 0, 5, 50 or 250 μM and 5-BDBD, A804598 or MRS2279 (Tocris Bioscience, UK) was used where indicated to block P2X₄, P2X₇ or P2Y₁ receptors, respectively. Cells were cultured during 24, 48 or 96 hrs and analyzed by flow cytometry. Supernatants were collected after 96 hrs and cytokine production was analyzed by ELISA.

For cultivation of human Treg cells, recombinant human IL-2 (EBIOSCIENCE) and superparamagnetic beads coupled with human α-CD3 and α-CD28 (LIFE TECHNOLOGIES, Carlsbad, Calif.) were used. Cells were cultured during 10 days and analyzed by flow cytometry. Supernatants were collected after 96 hrs and cytokine production was analyzed by ELISA.

Flow cytometry

Fluorescently labeled anti-mouseα-CD4 (GK1.5), α-CD25 (PC61), α-CD11c (HL3), α-IFN-γ (XMG1.2) and unlabeled α-CD16/CD32 (2.4G2) antibodies were obtained from BD BIOSCIENCES (San Jose, Calif.). Fluorescently labeled anti-mouse α-Foxp3 (FJK-16s), α-IL-17A (eBiol7B7), α-IL-10 (JES5-16E3) were obtained from EBIOSCIENCE (San Diego, Calif.). Fluorescently labeled anti-human α-CD4 (OKT4), α-CD25 (BC96), α-Foxp3 (PCH101), and α-IL-17A (eBio64DEC17) were all obtained from EBIOSCIENCE.

Intracellular staining for Foxp3, IL-17A, and IL-10 was performed according to manufacturers' protocols. Splenocytes were re-stimulated in complete media (HL-1 media containing 10% FCS, 1% L-Glutamine, 1% Penicillin/Streptomycin; all BIO WHITTAKER, Walkersville, Md.) for 4 hours at 37° C. with ionomycin (500 ng/ml) and phorbol 12-myristate 13-acetate (50 ng/ml, both SIGMA-ALDRICH, St. Louis, Mo.). Brefeldin A (BD BIOSCIENCES) was added at a concentration of 0.67 μl/ml. Cells were fixed and permeabilized using Cytofix/Cytoperm solution (BD BIOSCIENCES) or Foxp3 fixation/permeabilization solution (EBIOSCIENCE), respectively. Apoptosis staining with fluorescently labeled Annexin V (BD BIOSCIENCE) and proliferation assay with Carboxyfluorescein diacetate succinimidyl ester (INVITROGEN, Carlsbad, Calif.) were both performed according to manufacturers' protocols using commercial kits. Flow cytometry measurements of single-cell suspensions were performed on a FACSCALIBUR using standard procedures and data were analyzed using FLOWJO software (TREE STAR, Ashland, Oreg.).

### ELISA

Mouse IL-4, IL-6, IL-10, IL-17A, IFN-γ and human IL-17A and IL-17F were measured using commercial kits (EBIOSCIENCE). Briefly, ELISA plates were coated with 100 μl of anti-cytokine capture antibody at 4° C. overnight. Plates were then washed ×5 with 0.05% PBS-Tween (PBST) and coated for 1 hr with blocking buffer provided by the manufacturer. Samples or standards were added in triplicates (100 μl/well) and incubated at 4° C. overnight. Wells were washed ×5 with PBST and incubated with 100 μl of anti-cytokine detection antibody at 4° C. overnight. Wells were then washed ×5 with PBST and incubated with 100 μl of avidin-HRP at room temperature for 30 min. Thereafter, wells were washed ×7 with PBST and incubated with 100 μl/well of a substrate. The reaction was stopped after 15 min with 1M H₂SO₄ and absorbance was measured using a multiplate microplate fluorescence reader (SYNERGY HT, BIOTEK) at 405 nm.

### RNA Extraction and Quantitative PCR

RNA extraction from isolated Treg cells after cultivation was performed using the RNAqueous extraction kit according to the manufacturer's protocols (APPLIED BIOSYSTEMS, Carlsbad, Calif.). Briefly, Treg cells were homogenized in lysis buffer (total volume of 0.5 ml) and passed through a column. After successive washes, RNA was eluted and reverse transcription was performed using i-Script cDNA synthesis kit (BIO-RAD LABORATORIES, Hercules, Calif.). PCR reactions were performed with Taqman primers and probes from Applied Biosystems. The housekeeping gene GAPDH was used as control. Relative gene expression was determined as described previously⁴⁹.

### Microscopy, Deconvolution, 3D Reconstruction.

Cultured T cells were stained on ice with 2 μg/ml of either control, anti-P2RX4 or anti-P2X7 in the presence of 2% IgG-free BSA (Life Technology) for 20 mins, washed and incubated in the presence of the 0.01% Hoescht 33342 with relevant Alexa 488 secondary Ab. After staining, cells were washed three times, mounted in fluorescence mounting media (DAKOCYTOMATION, Carpinteria, Calif.), and imagedusing an Olympus BX62 motorized microscope fitted with a cooled Hamamatsu Orca AGCCD camera. The microscope, filters, and camera were controlled by SLIDE BOOK 5.0 (3I) Acquired Z-stacks were further processed using the deconvolution module of Volocity 5.0 (IMPROVISION, Waltham, Mass.) followed by 3-D surface rendering reconstruction using maximum intensity projection algorithms.

### Skin Transplantation Model

Full-thickness tail skin grafts (˜1 cm²) were procured from C57BL/6 mice and engrafted onto the dorsolateral thoracic wall of DBA/2 recipient mice using interrupted 5-0 Vicryl sutures. NAD⁺ (100 μl of a solution containing 10 mg in PBS), or PBS (100 μl) were injected intraperitoneally and grafts were covered with gauze and adhesive bandage for 5 days. Graft survival was then monitored daily and rejection was defined as graft necrosis of 100%. Two investigators blinded for the particular experimental groups assessed graft survival independently. Eight days after transplantation, single-cell leukocyte suspensions were obtained from spleens procured from recipient mice to perform re-stimulation (with PMA and Ionomycin for 2hrs) and staining for surface and intracellular antigens as described above.

### Statistical Analysis

Values and error bars represent mean ± SEM. Data were analyzed using GRAPHPAD PRISM (GRAPHPAD Software, San Diego, Calif.). Log-rank test was used to compare survival curves and unpaired two-tailed Student's t-test was used for all other results. A p value <0.05 was considered statistically significant.

### REFERENCES

1. Fontenot, J. D., Gavin, M. A. & Rudensky, A. Y. Foxp3 programs the development and function of CD4+CD25+ regulatory T cells. Nat Immunol 4, 330-336 (2003).
2. Hori, S., Nomura, T. & Sakaguchi, S. Control of regulatory T cell development by the transcription factor Foxp3. Science 299, 1057-1061 (2003).
3. Josefowicz, S. Z., Lu, L. F. & Rudensky, A. Y. Regulatory T cells: mechanisms of differentiation and function. Annu Rev Immunol 30, 531-564 (2012).
4. Zhou, X., et al. Instability of the transcription factor Foxp3 leads to the generation of pathogenic memory T cells in vivo. Nat Immunol 10, 1000-1007 (2009).
5. Lee, Y. K., Mukasa, R., Hatton, R. D. & Weaver, C. T. Developmental plasticity of Th17 and Treg cells. Curr Opin Immunol 21, 274-280 (2009).
6. Zhu, J., Yamane, H. & Paul, W. E. Differentiation of effector CD4 T cell populations (*). Annu Rev Immunol 28, 445-489 (2010).
7. Hawiger, D. & Flavell, R. A. Regulatory T cells that become autoaggressive. Nat Immunol 10, 938-939 (2009).
8. Yang, X. O., et al. Molecular antagonism and plasticity of regulatory and inflammatory T cell programs. Immunity 29, 44-56 (2008).
9. Xu, L., Kitani, A., Fuss, I. & Strober, W. Cutting edge: regulatory T cells induce CD4+CD25−Foxp3− T cells or are self-induced to become Th17 cells in the absence of exogenous TGF-beta. J Immunol 178, 6725-6729 (2007).
10. Voo, K. S., et al. Identification of IL-17-producing FOXP3+ regulatory T cells in humans. Proc Natl Acad Sci U S A 106, 4793-4798 (2009).
11. Ivanov, I I, et al. The orphan nuclear receptor RORgammat directs the differentiation program of proinflammatory IL-17+ T helper cells. Cell 126, 1121-1133 (2006).
12. Cua, D. J., et al. Interleukin-23 rather than interleukin-12 is the critical cytokine for autoimmune inflammation of the brain. Nature 421, 744-748 (2003).
13. Bettelli, E., et al. Reciprocal developmental pathways for the generation of pathogenic effector TH17 and regulatory T cells. Nature 441, 235-238 (2006).
14. Veldhoen, M., Hocking, R. J., Atkins, C. J., Locksley, R. M. & Stockinger, B. TGFbeta in the context of an inflammatory cytokine milieu supports de novo differentiation of IL-17-producing T cells. Immunity 24, 179-189 (2006).
15. Martinez, G. J., Nurieva, R. I., Yang, X. O. & Dong, C. Regulation and function of proinflammatory TH17 cells. Ann N Y Acad Sci 1143, 188-211 (2008).
16. Laurence, A., et al. Interleukin-2 signaling via STAT5 constrains T helper 17 cell generation. Immunity 26, 371-381 (2007).
17. Liao, W., Lin, J. X., Wang, L., Li, P. & Leonard, W. J. Modulation of cytokine receptors by IL-2 broadly regulates differentiation into helper T cell lineages. Nat Immunol 12, 551-559 (2011).
18. Mosmann, T. R., Cherwinski, H., Bond, M. W., Giedlin, M. A. & Coffman, R. L. Two types of murine helper T cell clone. I. Definition according to profiles of lymphokine activities and secreted proteins. J Immunol 136, 2348-2357 (1986).
19. Mosmann, T. R. & Coffman, R. L. TH1 and TH2 cells: different patterns of lymphokine secretion lead to different functional properties. Annu Rev Immunol 7, 145-173 (1989).
20. Dong, C. & Flavell, R. A. Control of T helper cell differentiation—in search of master genes. Sci STKE 2000, pe 1 (2000).
21. Mutafova-Yambolieva, V. N., et al. Beta-nicotinamide adenine dinucleotide is an inhibitory neurotransmitter in visceral smooth muscle. Proc Natl Acad Sci U S A 104, 16359-16364 (2007).
22. Breen, L. T., Smyth, L. M., Yamboliev, I. A. & Mutafova-Yambolieva, V. N. beta-NAD is a novel nucleotide released on stimulation of nerve terminals in human urinary bladder detrusor muscle. Am J Physiol Renal Physiol 290, F486-495 (2006).
23. Hwang, S. J., et al. beta-nicotinamide adenine dinucleotide is an enteric inhibitory neurotransmitter in human and nonhuman primate colons. Gastroenterology 140, 608-617 e606 (2011).
24. Durnin, L., et al. Release, neuronal effects and removal of extracellular beta-nicotinamide adenine dinucleotide (beta-NAD(+)) in the rat brain. Eur J Neurosci 35, 423-435 (2012).
25. Smyth, L. M., Bobalova, J., Mendoza, M. G., Lew, C. & Mutafova-Yambolieva, V. N. Release of beta-nicotinamide adenine dinucleotide upon stimulation of postganglionic nerve terminals in blood vessels and urinary bladder. J Biol Chem 279, 48893-48903 (2004).
26. Penberthy, W. T. & Tsunoda, I. The importance of NAD in multiple sclerosis. Curr Pharm Des 15, 64-99 (2009).
27. Billington, R. A., et al. Emerging functions of extracellular pyridine nucleotides. Mol Med 12, 324-327 (2006).
28. Bruzzone, S., Guida, L., Zocchi, E., Franco, L. & De Flora, A. Connexin 43 hemi channels mediate Ca2+-regulated transmembrane NAD+ fluxes in intact cells. Faseb J 15, 10-12 (2001).
29. Schenk, U., et al. ATP inhibits the generation and function of regulatory T cells through the activation of purinergic P2X receptors. Sci Signal 4, ra 12 (2011).
30. Schwarz, N., et al. Activation of the P2X7 ion channel by soluble and covalently bound ligands. Purinergic Signal 5, 139-149 (2009).
31. Seman, M., et al. NAD-induced T cell death: ADP-ribosylation of cell surface proteins by ART2 activates the cytolytic P2X7 purinoceptor. Immunity 19, 571-582 (2003).
32. Hubert, S., et al. Extracellular NAD+ shapes the Foxp3+ regulatory T cell compartment through the ART2-P2X7 pathway. J Exp Med 207, 2561-2568 (2010).
33. Laurence, A., et al. STAT3 transcription factor promotes instability of nTreg cells and limits generation of iTreg cells during acute murine graft-versus-host disease. Immunity 37, 209-222 (2012).
34. Acosta-Rodriguez, E. V., Napolitani, G., Lanzavecchia, A. & Sallusto, F. Interleukins 1beta and 6 but not transforming growth factor-beta are essential for the differentiation of interleukin 17-producing human T helper cells. Nat Immunol 8, 942-949 (2007).
35. Manel, N., Unutmaz, D. & Littman, D. R. The differentiation of human T(H)-17 cells requires transforming growth factor-beta and induction of the nuclear receptor RORgammat. Nat Immunol 9, 641-649 (2008).
36. Stockinger, B. Good for Goose, but not for Gander: IL-2 interferes with Th17 differentiation. Immunity 26, 278-279 (2007).
37. Yang, L., et al. IL-21 and TGF-beta are required for differentiation of human T(H)17 cells. Nature 454, 350-352 (2008).
38. Volpe, E., et al. A critical function for transforming growth factor-beta, interleukin 23 and proinflammatory cytokines in driving and modulating human T(H)-17 responses. Nat Immunol 9, 650-657 (2008).
39. Junger, W. G. Immune cell regulation by autocrine purinergic signaling. Nat Rev Immunol 11, 201-212 (2011).
40. Casati, A., et al. Cell-autonomous regulation of hematopoietic stem cell cycling activity by ATP. Cell Death Differ 18, 396-404 (2011).
41. Donnelly-Roberts, D. L., et al. [3H]A-804598 ([3H]2-cyano-1-[(1S)-1-phenylethyl]-3-quinolin-5-ylguanidine) is a novel, potent, and selective antagonist radioligand for P2X7 receptors. Neuropharmacology 56, 223-229 (2009).
42. Able, S. L., et al. Receptor localization, native tissue binding and ex vivo occupancy for centrally penetrant P2X7 antagonists in the rat. Br J Pharmacol 162, 405-414 (2011).
43. Boyer, J. L., Adams, M., Ravi, R. G., Jacobson, K. A. & Harden, T. K. 2-Chloro N(6)-methyl-(N)-methanocarba-2′-deoxyadenosine-3′,5′-bisphosphate is a selective high affinity P2Y(1) receptor antagonist. Br J Pharmacol 135, 2004-2010 (2002).
44. Hanidziar, D. & Koulmanda, M. Inflammation and the balance of Treg and Th17 cells in transplant rejection and tolerance. Curr Opin Organ Transplant 15, 411-415 (2010).
45. Heidt, S., Segundo, D. S., Chadha, R. & Wood, K. J. The impact of Th17 cells on transplant rejection and the induction of tolerance. Curr Opin Organ Transplant 15, 456-461 (2010).
46. Asadullah, K., Sterry, W. & Volk, H. D. Interleukin-10 therapy—review of a new approach. Pharmacol Rev 55, 241-269 (2003).
47. Okahashi, N., et al. Oral immunization of interleukin-4 (IL-4) knockout mice with a recombinant Salmonella strain or cholera toxin reveals that CD4+ Th2 cells producing IL-6 and IL-10 are associated with mucosal immunoglobulin A responses. Infect Immun 64, 1516-1525 (1996).
48. Magram, J., et al. IL-12-deficient mice are defective in IFN gamma production and type 1 cytokine responses. Immunity 4, 471-481 (1996).
49. Elkhal, A., et al. CD1d restricted natural killer T cells are not required for allergic skin inflammation. J Allergy Clin Immunol 118, 1363-1368 (2006).

### Example 3

### NAD+ as a Treatment for Allergy or Autoimmune Disease

### NAD+ Targets CD4+CD44^{high}CD62L^{high} Central/Memory Antigen Specific T Cells

When exposed to antigen, naïve CD4+ T cells undergo rapid proliferation and differentiate into effector cells that have the ability to infiltrate tissue and destroy pathogen invasion. After clearance, effector cells enter cell death and only a small fraction of cells with memory function remains. CD4+CD44^{high}CD62L^{high} central/memory T cells can produce a robust immune response when re-exposed to the same antigen. Activated/memory T cells have been shown to play an important role in many diseases including allergy and transplantation. Increasing evidence indicates that memory CD44^{high}CD4+ T cells induced lethal GVHD or transplant rejection.

It is understood in a clinical setting that immunosuppressants are unable to specifically target activated/memory CD4+ T cells. Thus, the inventors next investigated the specificity of NAD+ using an allergic mouse model consisting of several intraperitoneal (i.p.) injections of ovalbumin (OVA) with Alumn. As a control, a group of mice received a placebo solution (PBS). In addition, to assess the specific effect of NAD+ on CD4+CD44^{high}CD62L^{high} activated/memory T cells, a group of mice received OVA followed by NAD+ injection. After 21 days, CD4+CD44^{high}CD62L^{high} activated/memory T cell frequency was assessed and the data showed that NAD+ targets specifically CD4+CD44^{high}CD62L^{high} antigen specific cells.

Collectively, these results indicate that NAD+ targets memory antigen specific CD4+ T cells and can be a better treatment for allergy, autoimmune diseases or immunosuppressive agents than those that are currently administered to patients.

### Example 4

### NAD+ as a Treatment for Type 1 Diabetes

NOD/Schilt mice were used to assess the role of NAD+ as a treatment for Type I Diabetes (T1D). By the 20th week, levels of insulin started consistently rising above the normal average. By week 25, levels of insulin were detected over 600 mg/dL. These levels were consistently high for 1 week before treatment with NAD+ was initiated. Treatment with NAD+ was given daily at 40 mg per mouse. 5 days after treatment with NAD+, insulin levels started to decline at a significant rate, with one mouse almost reaching normal insulin levels, before the experiment was terminated.

It is important to note that most T1D studies begin treating diabetic animals when glucose levels reach 250 mg/dl or greater for 2 consecutive days. In the present study, the inventors waited until the mice had a level higher than 600 mg/dl. Even after the development of more serious disease, the inventors were able to reduce the blood glucose levels from high to much lower levels (almost normal levels for at least one of the treated mice). In addition, the type 1 diabetic mice exhibited notable lethargy but became much more active and aggressive within 10 days of NAD+ treatment.

Taken together, these results indicate that NAD+ can be used in the treatment and/or reversal of Type 1 Diabetes.

1. A method for treating or preventing an immune disease, the method comprising administering a composition comprising a therapeutically effective amount of NAD+ to a subject in need thereof, thereby treating the immune disease.

2-38. (canceled)

39. The method of claim 1, wherein the composition further comprises a pharmaceutically acceptable carrier.

40. The method of claim 1, wherein the immune disease is an autoimmune disease.

41. The method of claim 1, wherein the immune disease is selected from the group consisting of Type 1 diabetes, allergy, asthma, eczema, systemic lupus erythematosus, rheumatoid arthritis, transplantation, inflammatory bowel disease, cancer, multiple sclerosis and sepsis.

42. The method of claim 1, wherein the composition is administered by a route selected from the group consisting of: intravenous, intramuscular, subcutaneous, intradermal, topical, intraperitoneal, intrathecal, intrapleural, intrauterine, rectal, vaginal, intrasynovial, intraorgan, intraocular/periocular, intratumor, and parenteral administration.

43. The method of claim 1, further comprising a step of diagnosing the subject with an immune disease prior to treatment.

44. The method of claim 1, further comprising a step of measuring NAD+ in the subject prior to treatment.

45. The method of claim 44, wherein the amount of NAD+ is compared to a reference value.

46. A method for activating CD4+ helper T cells, the method comprising contacting a CD4+ helper T cell with a composition comprising an effective amount of NAD+ to a subject in need thereof, thereby activating the CD4+ helper T cell.

47. An assay comprising:
a. measuring or quantifying the amount of NAD+ in a biological sample obtained from a subject having or suspected of having an immune disease; and
b. comparing the measured or quantified amount of NAD+ with a reference value, and if the amount of NAD+ is decreased relative to the reference value, identifying the subject as having an increased probability of having an immune disease.

48. The assay of claim 47, wherein the immune disease is an autoimmune disease.

49. The assay of claim 47, wherein the immune disease is selected from the group consisting of Type 1 diabetes, allergy, asthma, eczema, systemic lupus erythematosus, rheumatoid arthritis, transplantation, inflammatory bowel disease, cancer, multiple sclerosis and sepsis.

50. The assay of claim 47, further comprising a step of treating the subject.

51. The assay of claim 50, wherein the subject is treated with a composition comprising a therapeutically effective amount of NAD+.

52. The assay of claim 47, wherein the amount of NAD+ is compared to a reference value.

53. The assay of claim 52, wherein the reference value is obtained from a plurality of subjects having an immune disease or from a plurality of subjects in which an immune disease cannot be detected using standard methods.

54. A method for diagnosing an immune disease in a subject, the method comprising:
(a) measuring the amount of NAD+ in a biological sample obtained from a subject suspected of having an immune disease, and
(b) comparing the amount of NAD+ measured in the biological sample to the amount of NAD+ in a reference sample, wherein a decrease in the amount of NAD+ compared to the reference value indicates that the subject has an immune disease.

55. The method of claim 54, further comprising a step of treating the subject.

56. The method of claim 55, wherein the subject is treated with a composition comprising a therapeutically effective amount of NAD+.

57. The method of claim 54, wherein the amount of NAD+ is compared to a reference value.

58. The method of claim 57, wherein the reference value is obtained from a plurality of subjects in which an immune disease cannot be detected using standard methods.

59. The method of claim 54, wherein the immune disease is an autoimmune disease.
